SEQUENCE LISTING (351 sequences)

SEQ ID NO: 1 - DNA, 22 bp, Mus musculus
aggttacctaacggagcgagga

SEQ ID NO: 2 - DNA, 22 bp, Mus musculus
tgggtactggatcagtcttcca

SEQ ID NO: 3 - DNA, 23 bp, Mus musculus
tagttgctgagcgtcgtcaggga

SEQ ID NO: 4 - DNA, 22 bp, Mus musculus
cagcactccctgacgacgctca

SEQ ID NO: 5 - DNA, 23 bp, Mus musculus
cgagagacgcggcgcggccattg

SEQ ID NO: 6 - DNA, 23 bp, Mus musculus
ccgcgtctctcgcgggggggtgg

SEQ ID NO: 7 - DNA, 23 bp, Mus musculus
cgggagtggaaaggggcggggca

SEQ ID NO: 8 - DNA, 23 bp, Mus musculus
cccgcgttgtcggatcagcagac

SEQ ID NO: 9 - DNA, 22 bp, Mus musculus
acgcgggggagccgggagtgga

SEQ ID NO: 10 - DNA, 22 bp, Mus musculus
ccgacaacgcgggggagccggg

SEQ ID NO: 11 - DNA, 23 bp, Mus musculus
agaccgattctgggcgctgcgtc

SEQ ID NO: 12 - DNA, 23 bp, Mus musculus
gcccagaatcggtctgctgatcc

SEQ ID NO: 13 - DNA, 22 bp, Mus musculus
tgggcgctgcgtcgcatcggtg

SEQ ID NO: 14 - DNA, 22 bp, Mus musculus
gcgtcgcatcggtggcaggtaa

SEQ ID NO: 15 - DNA, 22 bp, Mus musculus
gcgacgcagcgcccagaatcgg

SEQ ID NO: 16 - DNA, 23 bp, Mus musculus
gatgcgacgcagcgcccagaatc

SEQ ID NO: 17 - DNA, 23 bp, Mus musculus
gatgcgacgcagcgcccagaatc

SEQ ID NO: 18 - DNA, 22 bp, Mus musculus
catcggtggcaggtaagcgggc

SEQ ID NO: 19 - DNA, 23 bp, Mus musculus
ggtaagcgggctgctgaagccag

SEQ ID NO: 20 - DNA, 23 bp, Mus musculus
aagcgggctgctgaagccaggcc

SEQ ID NO: 21 - DNA, 23 bp, Mus musculus
agcttgacgacggaaggctgagt

SEQ ID NO: 22 - DNA, 24 bp, Mus musculus
gccgagcttgacgacggaaggctg

SEQ ID NO: 23 - DNA, 23 bp, Mus musculus
cggctcactgcgcctctcggggc

SEQ ID NO: 24 - DNA, 22 bp, Mus musculus
gagccgagcttgacgacggaag

SEQ ID NO: 25 - DNA, 23 bp, Mus musculus
tctcggggccttgaggccacggg

SEQ ID NO: 26 - DNA, 23 bp, Mus musculus
gccttgaggccacggggactagg

SEQ ID NO: 27 - DNA, 22 bp, Mus musculus
gaggccacggggactaggactg

SEQ ID NO: 28 - DNA, 22 bp, Mus musculus
gagtctggctgggaggtgactg

SEQ ID NO: 29 - DNA, 22 bp, Mus musculus
ggctgggaggtgactgtacacc

SEQ ID NO: 30 - DNA, 22 bp, Mus musculus
gagtcgcgcagcagggggtgta

SEQ ID NO: 31 - DNA, 22 bp, Mus musculus
ctccaggagtcgcgcagcaggg

SEQ ID NO: 32 - DNA, 22 bp, Mus musculus
cggttcctccaggagtcgcgca

SEQ ID NO: 33 - DNA, 22 bp, Mus musculus
cggttcctccaggagtcgcgca

SEQ ID NO: 34 - DNA, 22 bp, Mus musculus
gggattcggttcctccaggagt

SEQ ID NO: 35 - DNA, 23 bp, Mus musculus
ggaaggaaaggctggctcccggc

SEQ ID NO: 36 - DNA, 23 bp, Mus musculus
gggagtaatgactgtggtggaat

SEQ ID NO: 37 - protein, 7 aa, Artificial Sequence
  source: /note=“Description of Artificial Sequence Synthetic peptide”
RSDNLAR

SEQ ID NO: 38 - protein, 7 aa, Artificial Sequence
  source: /note=“Description of Artificial Sequence Synthetic peptide”
TPSYLPT

SEQ ID NO: 39 - protein, 7 aa, Artificial Sequence
  source: /note=“Description of Artificial Sequence Synthetic peptide”
RSDHLSA

SEQ ID NO: 40 - protein, 7 aa, Artificial Sequence
  source: /note=“Description of Artificial Sequence Synthetic peptide”
QESDLVR

SEQ ID NO: 41 - protein, 7 aa, Artificial Sequence
  source: /note=“Description of Artificial Sequence Synthetic peptide”
SNQNLTT

SEQ ID NO: 42 - protein, 7 aa, Artificial Sequence
  source: /note=“Description of Artificial Sequence Synthetic peptide”
LRHHLTR

SEQ ID NO: 43 - protein, 7 aa, Artificial Sequence
  source: /note=“Description of Artificial Sequence Synthetic peptide”
RSAHLSR

SEQ ID NO: 44 - protein, 7 aa, Artificial Sequence
  source: /note=“Description of Artificial Sequence Synthetic peptide”
RSDNLSE

SEQ ID NO: 45 - protein, 7 aa, Artificial Sequence
  source: /note=“Description of Artificial Sequence Synthetic peptide”
RSDSLLR

SEQ ID NO: 46 - protein, 7 aa, Artificial Sequence
  source: /note=“Description of Artificial Sequence Synthetic peptide”
RSDTLSQ

SEQ ID NO: 47 - protein, 7 aa, Artificial Sequence
  source: /note=“Description of Artificial Sequence Synthetic peptide”
RSDDLTR

SEQ ID NO: 48 - protein, 7 aa, Artificial Sequence
  source: /note=“Description of Artificial Sequence Synthetic peptide”
RSDDLTR

SEQ ID NO: 49 - protein, 7 aa, Artificial Sequence
  source: /note=“Description of Artificial Sequence Synthetic peptide”
RSDHLST

SEQ ID NO: 50 - protein, 7 aa, Artificial Sequence
  source: /note=“Description of Artificial Sequence Synthetic peptide”
LRHHLTR

SEQ ID NO: 51 - protein, 7 aa, Artificial Sequence
  source: /note=“Description of Artificial Sequence Synthetic peptide”
DRSALAR

SEQ ID NO: 52 - protein, 7 aa, Artificial Sequence
  source: /note=“Description of Artificial Sequence Synthetic peptide”
QSGNLAR

SEQ ID NO: 53 - protein, 7 aa, Artificial Sequence
  source: /note=“Description of Artificial Sequence Synthetic peptide”
QSGNLAR

SEQ ID NO: 54 - protein, 7 aa, Artificial Sequence
  source: /note=“Description of Artificial Sequence Synthetic peptide”
RSDHLSE

SEQ ID NO: 55 - protein, 7 aa, Artificial Sequence
  source: /note=“Description of Artificial Sequence Synthetic peptide”
WNSDLRK

SEQ ID NO: 56 - protein, 7 aa, Artificial Sequence
  source: /note=“Description of Artificial Sequence Synthetic peptide”
RSAHLSR

SEQ ID NO: 57 - protein, 7 aa, Artificial Sequence
  source: /note=“Description of Artificial Sequence Synthetic peptide”
QSAHRKN

SEQ ID NO: 58 - protein, 7 aa, Artificial Sequence
  source: /note=“Description of Artificial Sequence Synthetic peptide”
DRSHLTR

SEQ ID NO: 59 - protein, 7 aa, Artificial Sequence
  source: /note=“Description of Artificial Sequence Synthetic peptide”
RSDHLSR

SEQ ID NO: 60 - protein, 7 aa, Artificial Sequence
  source: /note=“Description of Artificial Sequence Synthetic peptide”
DRSHLTR

SEQ ID NO: 61 - protein, 7 aa, Artificial Sequence
  source: /note=“Description of Artificial Sequence Synthetic peptide”
RSDTLST

SEQ ID NO: 62 - protein, 7 aa, Artificial Sequence
  source: /note=“Description of Artificial Sequence Synthetic peptide”
QKGTLGE

SEQ ID NO: 63 - protein, 7 aa, Artificial Sequence
  source: /note=“Description of Artificial Sequence Synthetic peptide”
DRSNLSR

SEQ ID NO: 64 - protein, 7 aa, Artificial Sequence
  source: /note=“Description of Artificial Sequence Synthetic peptide”
DRSNLSR

SEQ ID NO: 65 - protein, 7 aa, Artificial Sequence
  source: /note=“Description of Artificial Sequence Synthetic peptide”
QKGTLGE

SEQ ID NO: 66 - protein, 7 aa, Artificial Sequence
  source: /note=“Description of Artificial Sequence Synthetic peptide”
RSDVLSA

SEQ ID NO: 67 - protein, 7 aa, Artificial Sequence
  source: /note=“Description of Artificial Sequence Synthetic peptide”
RSDNLSE

SEQ ID NO: 68 - protein, 7 aa, Artificial Sequence
  source: /note=“Description of Artificial Sequence Synthetic peptide”
RSADLTR

SEQ ID NO: 69 - protein, 7 aa, Artificial Sequence
  source: /note=“Description of Artificial Sequence Synthetic peptide”
RSADLTR

SEQ ID NO: 70 - protein, 7 aa, Artificial Sequence
  source: /note=“Description of Artificial Sequence Synthetic peptide”
QSGHLAR

SEQ ID NO: 71 - protein, 7 aa, Artificial Sequence
  source: /note=“Description of Artificial Sequence Synthetic peptide”
RSDTLSE

SEQ ID NO: 72 - protein, 7 aa, Artificial Sequence
  source: /note=“Description of Artificial Sequence Synthetic peptide”
QSGHLAR

SEQ ID NO: 73 - protein, 7 aa, Artificial Sequence
  source: /note=“Description of Artificial Sequence Synthetic peptide”
DDSHRKD

SEQ ID NO: 74 - protein, 7 aa, Artificial Sequence
  source: /note=“Description of Artificial Sequence Synthetic peptide”
DRSALAR

SEQ ID NO: 75 - protein, 7 aa, Artificial Sequence
  source: /note=“Description of Artificial Sequence Synthetic peptide”
DRSALAR

SEQ ID NO: 76 - protein, 7 aa, Artificial Sequence
  source: /note=“Description of Artificial Sequence Synthetic peptide”
DRSNLTR

SEQ ID NO: 77 - protein, 7 aa, Artificial Sequence
  source: /note=“Description of Artificial Sequence Synthetic peptide”
DRSHLAR

SEQ ID NO: 78 - protein, 7 aa, Artificial Sequence
  source: /note=“Description of Artificial Sequence Synthetic peptide”
RKYYLAK

SEQ ID NO: 79 - protein, 7 aa, Artificial Sequence
  source: /note=“Description of Artificial Sequence Synthetic peptide”
QSSDLRR

SEQ ID NO: 80 - protein, 7 aa, Artificial Sequence
  source: /note=“Description of Artificial Sequence Synthetic peptide”
KRCNLRC

SEQ ID NO: 81 - protein, 7 aa, Artificial Sequence
  source: /note=“Description of Artificial Sequence Synthetic peptide”
QRCHLTK

SEQ ID NO: 82 - protein, 7 aa, Artificial Sequence
  source: /note=“Description of Artificial Sequence Synthetic peptide”
HNSHRTK

SEQ ID NO: 83 - protein, 7 aa, Artificial Sequence
  source: /note=“Description of Artificial Sequence Synthetic peptide”
QSSDLRR

SEQ ID NO: 84 - protein, 7 aa, Artificial Sequence
  source: /note=“Description of Artificial Sequence Synthetic peptide”
QSSDLRR

SEQ ID NO: 85 - protein, 7 aa, Artificial Sequence
  source: /note=“Description of Artificial Sequence Synthetic peptide”
TNSNRIT

SEQ ID NO: 86 - protein, 7 aa, Artificial Sequence
  source: /note=“Description of Artificial Sequence Synthetic peptide”
QSGTRKT

SEQ ID NO: 87 - protein, 7 aa, Artificial Sequence
  source: /note=“Description of Artificial Sequence Synthetic peptide”
MACCRYA

SEQ ID NO: 88 - protein, 7 aa, Artificial Sequence
  source: /note=“Description of Artificial Sequence Synthetic peptide”
QRIDLTR

SEQ ID NO: 89 - protein, 7 aa, Artificial Sequence
  source: /note=“Description of Artificial Sequence Synthetic peptide”
QRIDLTR

SEQ ID NO: 90 - protein, 7 aa, Artificial Sequence
  source: /note=“Description of Artificial Sequence Synthetic peptide”
YSWRLSQ

SEQ ID NO: 91 - protein, 7 aa, Artificial Sequence
  source: /note=“Description of Artificial Sequence Synthetic peptide”
QSGNLAR

SEQ ID NO: 92 - protein, 7 aa, Artificial Sequence
  source: /note=“Description of Artificial Sequence Synthetic peptide”
DRSDLSR

SEQ ID NO: 93 - protein, 7 aa, Artificial Sequence
  source: /note=“Description of Artificial Sequence Synthetic peptide”
DSSHRTR

SEQ ID NO: 94 - protein, 7 aa, Artificial Sequence
  source: /note=“Description of Artificial Sequence Synthetic peptide”
QSGNLAR

SEQ ID NO: 95 - protein, 7 aa, Artificial Sequence
  source: /note=“Description of Artificial Sequence Synthetic peptide”
DRSARNS

SEQ ID NO: 96 - protein, 7 aa, Artificial Sequence
  source: /note=“Description of Artificial Sequence Synthetic peptide”
DRSNLTR

SEQ ID NO: 97 - protein, 7 aa, Artificial Sequence
  source: /note=“Description of Artificial Sequence Synthetic peptide”
DRSSRIK

SEQ ID NO: 98 - protein, 7 aa, Artificial Sequence
  source: /note=“Description of Artificial Sequence Synthetic peptide”
QSGHLSR

SEQ ID NO: 99 - protein, 7 aa, Artificial Sequence
  source: /note=“Description of Artificial Sequence Synthetic peptide”
ARWYLDK

SEQ ID NO: 100 - protein, 7 aa, Artificial Sequence
  source: /note=“Description of Artificial Sequence Synthetic peptide”
LKQHLTR

SEQ ID NO: 101 - protein, 7 aa, Artificial Sequence
  source: /note=“Description of Artificial Sequence Synthetic peptide”
WRSSLTA

SEQ ID NO: 102 - protein, 7 aa, Artificial Sequence
  source: /note=“Description of Artificial Sequence Synthetic peptide”
RNDHRIK

SEQ ID NO: 103 - protein, 7 aa, Artificial Sequence
  source: /note=“Description of Artificial Sequence Synthetic peptide”
KRCNLRC

SEQ ID NO: 104 - protein, 7 aa, Artificial Sequence
  source: /note=“Description of Artificial Sequence Synthetic peptide”
DRSALAR

SEQ ID NO: 105 - protein, 7 aa, Artificial Sequence
  source: /note=“Description of Artificial Sequence Synthetic peptide”
DRSALAR

SEQ ID NO: 106 - protein, 7 aa, Artificial Sequence
  source: /note=“Description of Artificial Sequence Synthetic peptide”
QKGTLGE

SEQ ID NO: 107 - protein, 7 aa, Artificial Sequence
  source: /note=“Description of Artificial Sequence Synthetic peptide”
HRRSRWG

SEQ ID NO: 108 - protein, 7 aa, Artificial Sequence
  source: /note=“Description of Artificial Sequence Synthetic peptide”
TSGHLSR

SEQ ID NO: 109 - protein, 7 aa, Artificial Sequence
  source: /note=“Description of Artificial Sequence Synthetic peptide”
RSDHLSE

SEQ ID NO: 110 - protein, 7 aa, Artificial Sequence
  source: /note=“Description of Artificial Sequence Synthetic peptide”
ERQTLIK

SEQ ID NO: 111 - protein, 7 aa, Artificial Sequence
  source: /note=“Description of Artificial Sequence Synthetic peptide”
HKQHRDA

SEQ ID NO: 112 - protein, 7 aa, Artificial Sequence
  source: /note=“Description of Artificial Sequence Synthetic peptide”
DRSNLSR

SEQ ID NO: 113 - protein, 7 aa, Artificial Sequence
  source: /note=“Description of Artificial Sequence Synthetic peptide”
RSADLTR

SEQ ID NO: 114 - protein, 7 aa, Artificial Sequence
  source: /note=“Description of Artificial Sequence Synthetic peptide”
RSDHLSE

SEQ ID NO: 115 - protein, 7 aa, Artificial Sequence
  source: /note=“Description of Artificial Sequence Synthetic peptide”
RSDNLSN

SEQ ID NO: 116 - protein, 7 aa, Artificial Sequence
  source: /note=“Description of Artificial Sequence Synthetic peptide”
DRSHLTR

SEQ ID NO: 117 - protein, 7 aa, Artificial Sequence
  source: /note=“Description of Artificial Sequence Synthetic peptide”
RSDTLSQ

SEQ ID NO: 118 - protein, 7 aa, Artificial Sequence
  source: /note=“Description of Artificial Sequence Synthetic peptide”
RSDHLSR

SEQ ID NO: 119 - protein, 7 aa, Artificial Sequence
  source: /note=“Description of Artificial Sequence Synthetic peptide”
DRSHLTR

SEQ ID NO: 120 - protein, 7 aa, Artificial Sequence
  source: /note=“Description of Artificial Sequence Synthetic peptide”
DRSHLTR

SEQ ID NO: 121 - protein, 7 aa, Artificial Sequence
  source: /note=“Description of Artificial Sequence Synthetic peptide”
QSGDLTR

SEQ ID NO: 122 - protein, 7 aa, Artificial Sequence
  source: /note=“Description of Artificial Sequence Synthetic peptide”
RSDVLSK

SEQ ID NO: 123 - protein, 7 aa, Artificial Sequence
  source: /note=“Description of Artificial Sequence Synthetic peptide”
DRSDLSR

SEQ ID NO: 124 - protein, 7 aa, Artificial Sequence
  source: /note=“Description of Artificial Sequence Synthetic peptide”
RSADLTR

SEQ ID NO: 125 - protein, 7 aa, Artificial Sequence
  source: /note=“Description of Artificial Sequence Synthetic peptide”
RSADLTR

SEQ ID NO: 126 - protein, 7 aa, Artificial Sequence
  source: /note=“Description of Artificial Sequence Synthetic peptide”
LRHHLTR

SEQ ID NO: 127 - protein, 7 aa, Artificial Sequence
  source: /note=“Description of Artificial Sequence Synthetic peptide”
QSSDLSR

SEQ ID NO: 128 - protein, 7 aa, Artificial Sequence
  source: /note=“Description of Artificial Sequence Synthetic peptide”
QSSNLAR

SEQ ID NO: 129 - protein, 7 aa, Artificial Sequence
  source: /note=“Description of Artificial Sequence Synthetic peptide”
DRSHLTR

SEQ ID NO: 130 - protein, 7 aa, Artificial Sequence
  source: /note=“Description of Artificial Sequence Synthetic peptide”
DRSDLSR

SEQ ID NO: 131 - protein, 7 aa, Artificial Sequence
  source: /note=“Description of Artificial Sequence Synthetic peptide”
QQSDLRV

SEQ ID NO: 132 - protein, 7 aa, Artificial Sequence
  source: /note=“Description of Artificial Sequence Synthetic peptide”
DRSNLSR

SEQ ID NO: 133 - protein, 7 aa, Artificial Sequence
  source: /note=“Description of Artificial Sequence Synthetic peptide”
RSDNLAR

SEQ ID NO: 134 - protein, 7 aa, Artificial Sequence
  source: /note=“Description of Artificial Sequence Synthetic peptide”
RSDTLST

SEQ ID NO: 135 - protein, 7 aa, Artificial Sequence
  source: /note=“Description of Artificial Sequence Synthetic peptide”
DRSNLSR

SEQ ID NO: 136 - protein, 7 aa, Artificial Sequence
  source: /note=“Description of Artificial Sequence Synthetic peptide”
QSGHLAR

SEQ ID NO: 137 - protein, 7 aa, Artificial Sequence
  source: /note=“Description of Artificial Sequence Synthetic peptide”
DRSNLSR

SEQ ID NO: 138 - protein, 7 aa, Artificial Sequence
  source: /note=“Description of Artificial Sequence Synthetic peptide”
RSDNLSE

SEQ ID NO: 139 - protein, 7 aa, Artificial Sequence
  source: /note=“Description of Artificial Sequence Synthetic peptide”
RSADLTR

SEQ ID NO: 140 - protein, 7 aa, Artificial Sequence
  source: /note=“Description of Artificial Sequence Synthetic peptide”
QSGHLAR

SEQ ID NO: 141 - protein, 7 aa, Artificial Sequence
  source: /note=“Description of Artificial Sequence Synthetic peptide”
QSGHLAR

SEQ ID NO: 142 - protein, 7 aa, Artificial Sequence
  source: /note=“Description of Artificial Sequence Synthetic peptide”
IRSTLRD

SEQ ID NO: 143 - protein, 7 aa, Artificial Sequence
  source: /note=“Description of Artificial Sequence Synthetic peptide”
RSDVLSE

SEQ ID NO: 144 - protein, 7 aa, Artificial Sequence
  source: /note=“Description of Artificial Sequence Synthetic peptide”
WRSCRSA

SEQ ID NO: 145 - protein, 7 aa, Artificial Sequence
  source: /note=“Description of Artificial Sequence Synthetic peptide”
QSANRTK

SEQ ID NO: 146 - protein, 7 aa, Artificial Sequence
  source: /note=“Description of Artificial Sequence Synthetic peptide”
QSGHLSR

SEQ ID NO: 147 - protein, 7 aa, Artificial Sequence
  source: /note=“Description of Artificial Sequence Synthetic peptide”
RSANLTR

SEQ ID NO: 148 - protein, 7 aa, Artificial Sequence
  source: /note=“Description of Artificial Sequence Synthetic peptide”
HNSSLKD

SEQ ID NO: 149 - protein, 7 aa, Artificial Sequence
  source: /note=“Description of Artificial Sequence Synthetic peptide”
RSDDLTR

SEQ ID NO: 150 - protein, 7 aa, Artificial Sequence
  source: /note=“Description of Artificial Sequence Synthetic peptide”
RNRDRIT

SEQ ID NO: 151 - protein, 7 aa, Artificial Sequence
  source: /note=“Description of Artificial Sequence Synthetic peptide”
TSSSRIN

SEQ ID NO: 152 - protein, 7 aa, Artificial Sequence
  source: /note=“Description of Artificial Sequence Synthetic peptide”
DRSALAR

SEQ ID NO: 153 - protein, 7 aa, Artificial Sequence
  source: /note=“Description of Artificial Sequence Synthetic peptide”
HNSHRTK

SEQ ID NO: 154 - protein, 7 aa, Artificial Sequence
  source: /note=“Description of Artificial Sequence Synthetic peptide”
QSSDLRR

SEQ ID NO: 155 - protein, 7 aa, Artificial Sequence
  source: /note=“Description of Artificial Sequence Synthetic peptide”
RLDWLPM

SEQ ID NO: 156 - protein, 7 aa, Artificial Sequence
  source: /note=“Description of Artificial Sequence Synthetic peptide”
RLDWLPM

SEQ ID NO: 157 - protein, 7 aa, Artificial Sequence
  source: /note=“Description of Artificial Sequence Synthetic peptide”
RRADLSR

SEQ ID NO: 158 - protein, 7 aa, Artificial Sequence
  source: /note=“Description of Artificial Sequence Synthetic peptide”
RSDDRIK

SEQ ID NO: 159 - protein, 7 aa, Artificial Sequence
  source: /note=“Description of Artificial Sequence Synthetic peptide”
RRTDLRR

SEQ ID NO: 160 - protein, 7 aa, Artificial Sequence
  source: /note=“Description of Artificial Sequence Synthetic peptide”
QSGDLTR

SEQ ID NO: 161 - protein, 7 aa, Artificial Sequence
  source: /note=“Description of Artificial Sequence Synthetic peptide”
QSGDLTR

SEQ ID NO: 162 - protein, 7 aa, Artificial Sequence
  source: /note=“Description of Artificial Sequence Synthetic peptide”
QSGTRKT

SEQ ID NO: 163 - protein, 7 aa, Artificial Sequence
  source: /note=“Description of Artificial Sequence Synthetic peptide”
HRSTRNR

SEQ ID NO: 164 - protein, 7 aa, Artificial Sequence
  source: /note=“Description of Artificial Sequence Synthetic peptide”
QSSDLRR

SEQ ID NO: 165 - protein, 7 aa, Artificial Sequence
  source: /note=“Description of Artificial Sequence Synthetic peptide”
DRSNLTR

SEQ ID NO: 166 - protein, 7 aa, Artificial Sequence
  source: /note=“Description of Artificial Sequence Synthetic peptide”
DRSNLTR

SEQ ID NO: 167 - protein, 7 aa, Artificial Sequence
  source: /note=“Description of Artificial Sequence Synthetic peptide”
RSADLSR

SEQ ID NO: 168 - protein, 7 aa, Artificial Sequence
  source: /note=“Description of Artificial Sequence Synthetic peptide”
HKWLRNQ

SEQ ID NO: 169 - protein, 7 aa, Artificial Sequence
  source: /note=“Description of Artificial Sequence Synthetic peptide”
HKSTLSD

SEQ ID NO: 170 - protein, 7 aa, Artificial Sequence
  source: /note=“Description of Artificial Sequence Synthetic peptide”
DRSSRIK

SEQ ID NO: 171 - protein, 7 aa, Artificial Sequence
  source: /note=“Description of Artificial Sequence Synthetic peptide”
DRSHLAR

SEQ ID NO: 172 - protein, 7 aa, Artificial Sequence
  source: /note=“Description of Artificial Sequence Synthetic peptide”
RKWTLQG

SEQ ID NO: 173 - protein, 7 aa, Artificial Sequence
  source: /note=“Description of Artificial Sequence Synthetic peptide”
LKQHLTR

SEQ ID NO: 174 - protein, 7 aa, Artificial Sequence
  source: /note=“Description of Artificial Sequence Synthetic peptide”
KRCNLRC

SEQ ID NO: 175 - protein, 7 aa, Artificial Sequence
  source: /note=“Description of Artificial Sequence Synthetic peptide”
DRSALAR

SEQ ID NO: 176 - protein, 7 aa, Artificial Sequence
  source: /note=“Description of Artificial Sequence Synthetic peptide”
QKGTLGE

SEQ ID NO: 177 - protein, 7 aa, Artificial Sequence
  source: /note=“Description of Artificial Sequence Synthetic peptide”
QKGTLGE

SEQ ID NO: 178 - protein, 7 aa, Artificial Sequence
  source: /note=“Description of Artificial Sequence Synthetic peptide”
HRSSLRR

SEQ ID NO: 179 - protein, 7 aa, Artificial Sequence
  source: /note=“Description of Artificial Sequence Synthetic peptide”
RNQHRKT

SEQ ID NO: 180 - protein, 7 aa, Artificial Sequence
  source: /note=“Description of Artificial Sequence Synthetic peptide”
DRSNLTR

SEQ ID NO: 181 - protein, 7 aa, Artificial Sequence
  source: /note=“Description of Artificial Sequence Synthetic peptide”
DNHDLIR

SEQ ID NO: 182 - protein, 7 aa, Artificial Sequence
  source: /note=“Description of Artificial Sequence Synthetic peptide”
ARSTRIT

SEQ ID NO: 183 - protein, 7 aa, Artificial Sequence
  source: /note=“Description of Artificial Sequence Synthetic peptide”
QSSDLSR

SEQ ID NO: 184 - protein, 7 aa, Artificial Sequence
  source: /note=“Description of Artificial Sequence Synthetic peptide”
DSSTRKT

SEQ ID NO: 185 - protein, 7 aa, Artificial Sequence
  source: /note=“Description of Artificial Sequence Synthetic peptide”
DRSNLSR

SEQ ID NO: 186 - protein, 7 aa, Artificial Sequence
  source: /note=“Description of Artificial Sequence Synthetic peptide”
QQSDLRV

SEQ ID NO: 187 - protein, 7 aa, Artificial Sequence
  source: /note=“Description of Artificial Sequence Synthetic peptide”
RSDSLLR

SEQ ID NO: 188 - protein, 7 aa, Artificial Sequence
  source: /note=“Description of Artificial Sequence Synthetic peptide”
TSGSLSR

SEQ ID NO: 189 - protein, 7 aa, Artificial Sequence
  source: /note=“Description of Artificial Sequence Synthetic peptide”
RSDHLSR

SEQ ID NO: 190 - protein, 7 aa, Artificial Sequence
  source: /note=“Description of Artificial Sequence Synthetic peptide”
IDHGRYR

SEQ ID NO: 191 - protein, 7 aa, Artificial Sequence
  source: /note=“Description of Artificial Sequence Synthetic peptide”
TSGNLTR

SEQ ID NO: 192 - protein, 7 aa, Artificial Sequence
  source: /note=“Description of Artificial Sequence Synthetic peptide”
TSGNLTR

SEQ ID NO: 193 - protein, 7 aa, Artificial Sequence
  source: /note=“Description of Artificial Sequence Synthetic peptide”
QSSDLSR

SEQ ID NO: 194 - protein, 7 aa, Artificial Sequence
  source: /note=“Description of Artificial Sequence Synthetic peptide”
QSGDLTR

SEQ ID NO: 195 - protein, 7 aa, Artificial Sequence
  source: /note=“Description of Artificial Sequence Synthetic peptide”
SEADRSR

SEQ ID NO: 196 - protein, 7 aa, Artificial Sequence
  source: /note=“Description of Artificial Sequence Synthetic peptide”
DRSNLSR

SEQ ID NO: 197 - protein, 7 aa, Artificial Sequence
  source: /note=“Description of Artificial Sequence Synthetic peptide”
DRSNLSR

SEQ ID NO: 198 - protein, 7 aa, Artificial Sequence
  source: /note=“Description of Artificial Sequence Synthetic peptide”
RSDVLSK

SEQ ID NO: 199 - protein, 7 aa, Artificial Sequence
  source: /note=“Description of Artificial Sequence Synthetic peptide”
RSDDLSR

SEQ ID NO: 200 - protein, 7 aa, Artificial Sequence
  source: /note=“Description of Artificial Sequence Synthetic peptide”
DRSHLTR

SEQ ID NO: 201 - protein, 7 aa, Artificial Sequence
  source: /note=“Description of Artificial Sequence Synthetic peptide”
DRSNLSR

SEQ ID NO: 202 - protein, 7 aa, Artificial Sequence
  source: /note=“Description of Artificial Sequence Synthetic peptide”
QSSDLSR

SEQ ID NO: 203 - protein, 7 aa, Artificial Sequence
  source: /note=“Description of Artificial Sequence Synthetic peptide”
DRSNRTT

SEQ ID NO: 204 - protein, 7 aa, Artificial Sequence
  source: /note=“Description of Artificial Sequence Synthetic peptide”
RSDNLAR

SEQ ID NO: 205 - protein, 7 aa, Artificial Sequence
  source: /note=“Description of Artificial Sequence Synthetic peptide”
RSAHLSR

SEQ ID NO: 206 - protein, 7 aa, Artificial Sequence
  source: /note=“Description of Artificial Sequence Synthetic peptide”
RSDNLAR

SEQ ID NO: 207 - protein, 7 aa, Artificial Sequence
  source: /note=“Description of Artificial Sequence Synthetic peptide”
YDYGRYT

SEQ ID NO: 208 - protein, 7 aa, Artificial Sequence
  source: /note=“Description of Artificial Sequence Synthetic peptide”
RWQYLPT

SEQ ID NO: 209 - protein, 7 aa, Artificial Sequence
  source: /note=“Description of Artificial Sequence Synthetic peptide”
QSGHLAR

SEQ ID NO: 210 - protein, 7 aa, Artificial Sequence
  source: /note=“Description of Artificial Sequence Synthetic peptide”
RSADLTR

SEQ ID NO: 211 - protein, 7 aa, Artificial Sequence
  source: /note=“Description of Artificial Sequence Synthetic peptide”
QSGHLAR

SEQ ID NO: 212 - protein, 7 aa, Artificial Sequence
  source: /note=“Description of Artificial Sequence Synthetic peptide”
IRSTLRD

SEQ ID NO: 213 - protein, 7 aa, Artificial Sequence
  source: /note=“Description of Artificial Sequence Synthetic peptide”
IRSTLRD

SEQ ID NO: 214 - protein, 7 aa, Artificial Sequence
  source: /note=“Description of Artificial Sequence Synthetic peptide”
RREDLIT

SEQ ID NO: 215 - protein, 7 aa, Artificial Sequence
  source: /note=“Description of Artificial Sequence Synthetic peptide”
QSGNLAR

SEQ ID NO: 216 - protein, 7 aa, Artificial Sequence
  source: /note=“Description of Artificial Sequence Synthetic peptide”
QSANRTK

SEQ ID NO: 217 - protein, 7 aa, Artificial Sequence
  source: /note=“Description of Artificial Sequence Synthetic peptide”
TSGSLTR

SEQ ID NO: 218 - protein, 7 aa, Artificial Sequence
  source: /note=“Description of Artificial Sequence Synthetic peptide”
LRHHLTR

SEQ ID NO: 219 - protein, 7 aa, Artificial Sequence
  source: /note=“Description of Artificial Sequence Synthetic peptide”
YKWTLRN

SEQ ID NO: 220 - protein, 7 aa, Artificial Sequence
  source: /note=“Description of Artificial Sequence Synthetic peptide”
QSATRTK

SEQ ID NO: 221 - protein, 7 aa, Artificial Sequence
  source: /note=“Description of Artificial Sequence Synthetic peptide”
WKESLGA

SEQ ID NO: 222 - protein, 7 aa, Artificial Sequence
  source: /note=“Description of Artificial Sequence Synthetic peptide”
RSADLSR

SEQ ID NO: 223 - protein, 7 aa, Artificial Sequence
  source: /note=“Description of Artificial Sequence Synthetic peptide”
QRCHLTK

SEQ ID NO: 224 - protein, 7 aa, Artificial Sequence
  source: /note=“Description of Artificial Sequence Synthetic peptide”
NSRNLRN

SEQ ID NO: 225 - protein, 7 aa, Artificial Sequence
  source: /note=“Description of Artificial Sequence Synthetic peptide”
QSSDLRR

SEQ ID NO: 226 - protein, 7 aa, Artificial Sequence
  source: /note=“Description of Artificial Sequence Synthetic peptide”
DRSNLTR

SEQ ID NO: 227 - protein, 7 aa, Artificial Sequence
  source: /note=“Description of Artificial Sequence Synthetic peptide”
DLTTLRK

SEQ ID NO: 228 - protein, 7 aa, Artificial Sequence
  source: /note=“Description of Artificial Sequence Synthetic peptide”
DLTTLRK

SEQ ID NO: 229 - protein, 7 aa, Artificial Sequence
  source: /note=“Description of Artificial Sequence Synthetic peptide”
DRSHLAR

SEQ ID NO: 230 - protein, 7 aa, Artificial Sequence
  source: /note=“Description of Artificial Sequence Synthetic peptide”
DTGARLK

SEQ ID NO: 231 - protein, 7 aa, Artificial Sequence
  source: /note=“Description of Artificial Sequence Synthetic peptide”
DRSNLTR

SEQ ID NO: 232 - protein, 7 aa, Artificial Sequence
  source: /note=“Description of Artificial Sequence Synthetic peptide”
VAEYRYK

SEQ ID NO: 233 - protein, 7 aa, Artificial Sequence
  source: /note=“Description of Artificial Sequence Synthetic peptide”
VAEYRYK

SEQ ID NO: 234 - protein, 7 aa, Artificial Sequence
  source: /note=“Description of Artificial Sequence Synthetic peptide”
RSDDRIK

SEQ ID NO: 235 - protein, 7 aa, Artificial Sequence
  source: /note=“Description of Artificial Sequence Synthetic peptide”
QSANRTK

SEQ ID NO: 236 - protein, 7 aa, Artificial Sequence
  source: /note=“Description of Artificial Sequence Synthetic peptide”
QSSDLRR

SEQ ID NO: 237 - protein, 7 aa, Artificial Sequence
  source: /note=“Description of Artificial Sequence Synthetic peptide”
HKWLRNQ

SEQ ID NO: 238 - protein, 7 aa, Artificial Sequence
  source: /note=“Description of Artificial Sequence Synthetic peptide”
QSTHRNA

SEQ ID NO: 239 - protein, 7 aa, Artificial Sequence
  source: /note=“Description of Artificial Sequence Synthetic peptide”
HRSSLRR

SEQ ID NO: 240 - protein, 7 aa, Artificial Sequence
  source: /note=“Description of Artificial Sequence Synthetic peptide”
DRSVLHR

SEQ ID NO: 241 - protein, 7 aa, Artificial Sequence
  source: /note=“Description of Artificial Sequence Synthetic peptide”
RSDDRKT

SEQ ID NO: 242 - protein, 7 aa, Artificial Sequence
  source: /note=“Description of Artificial Sequence Synthetic peptide”
HKSTLSD

SEQ ID NO: 243 - protein, 7 aa, Artificial Sequence
  source: /note=“Description of Artificial Sequence Synthetic peptide”
DRSHLAR

SEQ ID NO: 244 - protein, 7 aa, Artificial Sequence
  source: /note=“Description of Artificial Sequence Synthetic peptide”
DRSALAR

SEQ ID NO: 245 - protein, 7 aa, Artificial Sequence
  source: /note=“Description of Artificial Sequence Synthetic peptide”
RKWTLQG

SEQ ID NO: 246 - protein, 7 aa, Artificial Sequence
  source: /note=“Description of Artificial Sequence Synthetic peptide”
DRSALAR

SEQ ID NO: 247 - protein, 7 aa, Artificial Sequence
  source: /note=“Description of Artificial Sequence Synthetic peptide”
QKGTLGE

SEQ ID NO: 248 - protein, 7 aa, Artificial Sequence
  source: /note=“Description of Artificial Sequence Synthetic peptide”
HRSSLRR

SEQ ID NO: 249 - protein, 7 aa, Artificial Sequence
  source: /note=“Description of Artificial Sequence Synthetic peptide”
HRSSLRR

SEQ ID NO: 250 - protein, 7 aa, Artificial Sequence
  source: /note=“Description of Artificial Sequence Synthetic peptide”
TSSNLSR

SEQ ID NO: 251 - protein, 20 aa, Artificial Sequence
  source: /note=“Description of Artificial Sequence Synthetic peptide”
  SITE (1)..(20): /note=“This sequence may encompass 1-4 ‘Gly Gly Gly Gly Ser’ repeating units”
  source: /note=“See specification as filed for detailed description of substitutions and preferred embodiments”
GGGGSGGGGSGGGGSGGGGS

SEQ ID NO: 252 - protein, 5 aa, Artificial Sequence
  source: /note=“Description of Artificial Sequence Synthetic peptide”
DGGGS

SEQ ID NO: 253 - protein, 5 aa, Artificial Sequence
  source: /note=“Description of Artificial Sequence Synthetic peptide”
TGEKP

SEQ ID NO: 254 - protein, 9 aa, Artificial Sequence
  source: /note=“Description of Artificial Sequence Synthetic peptide”
LRQKDGERP

SEQ ID NO: 255 - protein, 4 aa, Artificial Sequence
  source: /note=“Description of Artificial Sequence Synthetic peptide”
GGRR

SEQ ID NO: 256 - protein, 8 aa, Artificial Sequence
  source: /note=“Description of Artificial Sequence Synthetic peptide”
GGRRGGGS

SEQ ID NO: 257 - protein, 9 aa, Artificial Sequence
  source: /note=“Description of Artificial Sequence Synthetic peptide”
LRQRDGERP

SEQ ID NO: 258 - protein, 12 aa, Artificial Sequence
  source: /note=“Description of Artificial Sequence Synthetic peptide”
LRQKDGGGSERP

SEQ ID NO: 259 - protein, 16 aa, Artificial Sequence
  source: /note=“Description of Artificial Sequence Synthetic peptide”
LRQKDGGGSGGGSERP

SEQ ID NO: 260 - protein, 6 aa, Artificial Sequence
  source: /note=“Description of Artificial Sequence Synthetic peptide”
TGSQKP

SEQ ID NO: 261 - protein, 96 aa, Homo sapiens
DAKSLTAWSRTLVTFKDVFVDFTREEWKLLDTAQQIVYRNVMLENYKNLVSLGYQLTKPDVILRLEKGEEPWLVEREIHQETHPDSETAFEIKSSV

SEQ ID NO: 262 - protein, 7 aa, Artificial Sequence
  source: /note=“Description of Artificial Sequence Synthetic peptide”
RSANLTR

SEQ ID NO: 263 - protein, 7 aa, Artificial Sequence
  source: /note=“Description of Artificial Sequence Synthetic peptide”
RQEHRVA

SEQ ID NO: 264 - protein, 7 aa, Artificial Sequence
  source: /note=“Description of Artificial Sequence Synthetic peptide”
QSADRTK

SEQ ID NO: 265 - protein, 7 aa, Artificial Sequence
  source: /note=“Description of Artificial Sequence Synthetic peptide”
DRSNRKT

SEQ ID NO: 266 - protein, 7 aa, Artificial Sequence
  source: /note=“Description of Artificial Sequence Synthetic peptide”
RNGHLLD

SEQ ID NO: 267 - protein, 7 aa, Artificial Sequence
  source: /note=“Description of Artificial Sequence Synthetic peptide”
WRSSLRA

SEQ ID NO: 268 - protein, 7 aa, Artificial Sequence
  source: /note=“Description of Artificial Sequence Synthetic peptide”
IKHDLHR

SEQ ID NO: 269 - protein, 7 aa, Artificial Sequence
  source: /note=“Description of Artificial Sequence Synthetic peptide”
DRSYRNT

SEQ ID NO: 270 - protein, 7 aa, Artificial Sequence
  source: /note=“Description of Artificial Sequence Synthetic peptide”
YRWLRNN

SEQ ID NO: 271 - protein, 7 aa, Artificial Sequence
  source: /note=“Description of Artificial Sequence Synthetic peptide”
QNANLMK

SEQ ID NO: 272 - protein, 7 aa, Artificial Sequence
  source: /note=“Description of Artificial Sequence Synthetic peptide”
RRSDLKR

SEQ ID NO: 273 - protein, 7 aa, Artificial Sequence
  source: /note=“Description of Artificial Sequence Synthetic peptide”
AKWNLDA

SEQ ID NO: 274 - DNA, 19 bp, Homo sapiens
tatgtaacggggagctgga

SEQ ID NO: 275 - DNA, 23 bp, Homo sapiens
atgagccacgtcagggagcgatg

SEQ ID NO: 276 - DNA, 22 bp, Homo sapiens
cacttgcatcagttgataccgc

SEQ ID NO: 277 - DNA, 23 bp, Homo sapiens
tttaagttaaagggtgcagcgct

SEQ ID NO: 278 - DNA, 22 bp, Homo sapiens
cggcgctcgcgggcggaggtcg

SEQ ID NO: 279 - DNA, 22 bp, Homo sapiens
acgcggggcggggaagggaagc

SEQ ID NO: 280 - DNA, 23 bp, Homo sapiens
gcctcggtcgtgaggagaggaga

SEQ ID NO: 281 - DNA, 22 bp, Homo sapiens
tgtaacggggagctggagcttt

SEQ ID NO: 282 - DNA, 22 bp, Homo sapiens
gggccgggaatgagtcaccgga

SEQ ID NO: 283 - DNA, 22 bp, Homo sapiens
cctccgagccagtcgctgacag

SEQ ID NO: 284 - DNA, 23 bp, Homo sapiens
ccgagccagtcgctgacagccgc

SEQ ID NO: 285 - protein, 7 aa, Artificial Sequence
  source: /note=“Description of Artificial Sequence Synthetic peptide”
YKCCLSD

SEQ ID NO: 286 - protein, 7 aa, Artificial Sequence
  source: /note=“Description of Artificial Sequence Synthetic peptide”
YPKDLAR

SEQ ID NO: 287 - protein, 7 aa, Artificial Sequence
  source: /note=“Description of Artificial Sequence Synthetic peptide”
KHSTRRV

SEQ ID NO: 288 - protein, 7 aa, Artificial Sequence
  source: /note=“Description of Artificial Sequence Synthetic peptide”
RSDALSE

SEQ ID NO: 289 - protein, 7 aa, Artificial Sequence
  source: /note=“Description of Artificial Sequence Synthetic peptide”
QLTHLNS

SEQ ID NO: 290 - protein, 7 aa, Artificial Sequence
  source: /note=“Description of Artificial Sequence Synthetic peptide”
RSSDRTK

SEQ ID NO: 291 - protein, 7 aa, Artificial Sequence
  source: /note=“Description of Artificial Sequence Synthetic peptide”
LRQDLKR

SEQ ID NO: 292 - protein, 7 aa, Artificial Sequence
  source: /note=“Description of Artificial Sequence Synthetic peptide”
QSSTRAR

SEQ ID NO: 293 - protein, 7 aa, Artificial Sequence
  source: /note=“Description of Artificial Sequence Synthetic peptide”
RSANLAR

SEQ ID NO: 294 - protein, 7 aa, Artificial Sequence
  source: /note=“Description of Artificial Sequence Synthetic peptide”
DRSALSR

SEQ ID NO: 295 - protein, 7 aa, Artificial Sequence
  source: /note=“Description of Artificial Sequence Synthetic peptide”
MRHHLDD

SEQ ID NO: 296 - protein, 7 aa, Artificial Sequence
  source: /note=“Description of Artificial Sequence Synthetic peptide”
QKGHLRK

SEQ ID NO: 297 - protein, 7 aa, Artificial Sequence
  source: /note=“Description of Artificial Sequence Synthetic peptide”
TNQNRIT

SEQ ID NO: 298 - protein, 7 aa, Artificial Sequence
  source: /note=“Description of Artificial Sequence Synthetic peptide”
DSSDRKK

SEQ ID NO: 299 - protein, 294 aa, Artificial Sequence
  source: /note=“Description of Artificial Sequence Synthetic polypeptide”
MAPKKKRKVGVPAAMAERPFQCRICMRNFSRSDNLARHIRTHTGEKPFACDICGRKFADDSHRKDHTKIHTGSQKPFQCRICMRNFSRSDHLSEHIRTHTGEKPFACDICGRKFAQSANRTKHTKIHTGSQKPFQCRICMRNFSDNHDLIRHIRTHTGEKPFACDICGRKFATSGSLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 300 - protein, 294 aa, Artificial Sequence
  source: /note=“Description of Artificial Sequence Synthetic polypeptide”
MAPKKKRKVGVPAAMAERPFQCRICMQNFSTPSYLPTHIRTHTGEKPFACDICGRKFADRSALARHTKIHTGSQKPFQCRICMQNFSERQTLIKHIRTHTGEKPFACDTCGRKFAQSGHLSRHTKIHTGSQKPFQCRICMQNFSARSTRITHIRTHTGEKPFACDICGRKFALRHHLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 301 - protein, 298 aa, Artificial Sequence
  source: /note=“Description of Artificial Sequence Synthetic polypeptide”
MAPKKKRKVGVPAAMAERPFQCRICMQNFSRSDHLSAHIRTHTGEKPFACDICGRKFADRSALARHTKIHTHPRAPIPKPFQCRICMQNFSHKQHRDAHIRTHTGEKPFACDICGRKFARSANLTRHTKIHTGSQKPFQCRICMQNFSQSSDLSRHIRTHTGEKPFACDICGRKFAYKWTLRNHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 302 - protein, 294 aa, Artificial Sequence
  source: /note=“Description of Artificial Sequence Synthetic polypeptide”
MAPKKKRKVGVPAAMAERPFQCRICMRNFSQESDLVRHIRTHTGEKPFACDICGRKFADRSNLTRHTKIHTGSQKPFQCRICMRNFSDRSNLSRHIRTHTGEKPFACDICGRKFAHNSSLKDHTKIHTGSQKPFQCRICMRNFSDSSTRKTHIRTHTGEKPFACDICGRKFAQSATRTKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 303 - protein, 298 aa, Artificial Sequence
  source: /note=“Description of Artificial Sequence Synthetic polypeptide”
MAPKKKRKVGVPAAMAERPFQCRICMRNFSSNQNLTTHIRTHTGEKPFACDICGRKFADRSHLARHTKIHTHPRAPIPKPFQCRICMRNFSRSADLTRHIRTHTGEKPFACDICGRKFARSDDLTRHTKIHTGSQKPFQCRICMRNFSDRSNLSRHIRTHTGEKPFACDICGRKFAWKESLGAHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 304 - protein, 298 aa, Artificial Sequence
  source: /note=“Description of Artificial Sequence Synthetic polypeptide”
MAPKKKRKVGVPAAMAERPFQCRICMRNFSLRHHLTRHIRTHTGEKPFACDICGRKFARKYYLAKHTKIHTGSQKPFQCRICMRNFSRSDHLSEHIRTHTGEKPFACDICGRKFARNRDRITHTKIHTHPRAPIPKPFQCRICMRNFSQQSDLRVHIRTHTGEKPFACDICGRKFARSADLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 305 - protein, 298 aa, Artificial Sequence
  source: /note=“Description of Artificial Sequence Synthetic polypeptide”
MAPKKKRKVGVPAAMAERPFQCRICMQNFSRSAHLSRHIRTHTGEKPFACDICGRKFAQSSDLRRHTKIHTHPRAPIPKPFQCRICMQNFSRSDNLSNHIRTHTGEKPFACDICGRKFATSSSRINHTKIHTGSQKPFQCRICMQNFSRSDSLLRHIRTHTGEKPFACDICGRKFAQRCHLTKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 306 - protein, 298 aa, Artificial Sequence
  source: /note=“Description of Artificial Sequence Synthetic polypeptide”
MAPKKKRKVGVPAAMAERPFQCRICMRNFSRSDNLSEHIRTHTGEKPFACDICGRKFAKRCNLRCHTKIHTHPRAPIPKPFQCRICMRNFSDRSHLTRHIRTHTGEKPFACDICGRKFADRSALARHTKIHTGSQKPFQCRICMRNFSTSGSLSRHIRTHTGEKPFACDICGRKFANSRNLRNHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 307 - protein, 294 aa, Artificial Sequence
  source: /note=“Description of Artificial Sequence Synthetic polypeptide”
MAPKKKRKVGVPAAMAERPFQCRICMQNFSRSDSLLRHIRTHTGEKPFACDICGRKFAQRCHLTKHTKIHTGSQKPFQCRICMQNFSRSDTLSQHIRTHTGEKPFACDICGRKFAHNSHRTKHTKIHTGSQKPFQCRICMQNFSRSDHLSRHIRTHTGEKPFACDICGRKFAQSSDLRRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 308 - protein, 294 aa, Artificial Sequence
  source: /note=“Description of Artificial Sequence Synthetic polypeptide”
MAPKKKRKVGVPAAMAERPFQCRICMQNFSRSDTLSQHIRTHTGEKPFACDICGRKFAHNSHRTKHTKIHTGSQKPFQCRICMQNFSRSDHLSRHIRTHTGEKPFACDICGRKFAQSSDLRRHTKIHTGSQKPFQCRICMQNFSIDHGRYRHIRTHTGEKPFACDICGRKFADRSNLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 309 - protein, 298 aa, Artificial Sequence
  source: /note=“Description of Artificial Sequence Synthetic polypeptide”
MAPKKKRKVGVPAAMAERPFQCRICMRNFSRSDDLTRHIRTHTGEKPFACDICGRKFAQSSDLRRHTKIHTGSQKPFQCRICMRNFSDRSHLTRHIRTHTGEKPFACDICGRKFARLDWLPMHTKIHTHPRAPIPKPFQCRICMRNFSTSGNLTRHIRTHTGEKPFACDICGRKFADLTTLRKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 310 - protein, 298 aa, Artificial Sequence
  source: /note=“Description of Artificial Sequence Synthetic polypeptide”
MAPKKKRKVGVPAAMAERPFQCRICMRNFSTSGNLTRHIRTHTGEKPFACDICGRKFALSQDLNRHTKIHTHPRAPIPKPFQCRICMRNFSDRSALSRHIRTHTGEKPFACDICGRKFARNDDRKKHTKIHTGSQKPFQCRICMRNFSQSGNLARHIRTHTGEKPFACDICGRKFAQRIDLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 311 - protein, 294 aa, Artificial Sequence
  source: /note=“Description of Artificial Sequence Synthetic polypeptide”
MAPKKKRKVGVPAAMAERPFQCRICMRNFSRSDHLSTHIRTHTGEKPFACDICGRKFATNSNRITHTKIHTGSQKPFQCRICMRNFSQSGDLTRHIRTHTGEKPFACDICGRKFARRADLSRHTKIHTGSQKPFQCRICMRNFSQSSDLSRHIRTHTGEKPFACDICGRKFADRSHLARHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 312 - protein, 294 aa, Artificial Sequence
  source: /note=“Description of Artificial Sequence Synthetic polypeptide”
MAPKKKRKVGVPAAMAERPFQCRICMRNFSLRHHLTRHIRTHTGEKPFACDICGRKFAQSGTRKTHTKIHTGSQKPFQCRICMRNFSRSDVLSKHIRTHTGEKPFACDICGRKFARSDDRIKHTKIHTGSQKPFQCRICMRNFSQSGDLTRHIRTHTGEKPFACDICGRKFADTGARLKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 313 - protein, 294 aa, Artificial Sequence
  source: /note=“Description of Artificial Sequence Synthetic polypeptide”
MAPKKKRKVGVPAAMAERPFQCRICMRNFSDRSALARHIRTHTGEKPFACDICGRKFAMACCRYAHTKIHTGSQKPFQCRICMRNFSDRSDLSRHIRTHTGEKPFACDICGRKFARRTDLRRHTKIHTGSQKPFQCRICMRNFSSEADRSRHIRTHTGEKPFACDICGRKFADRSNLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 314 - protein, 298 aa, Artificial Sequence
  source: /note=“Description of Artificial Sequence Synthetic polypeptide”
MAPKKKRKVGVPAAMAERPFQCRICMRNFSQSGNLARHIRTHTGEKPFACDICGRKFAQRIDLTRHTKIHTHPRAPIPKPFQCRICMRNFSRSADLTRHIRTHTGEKPFACDICGRKFAQSGDLTRHTKIHTGSQKPFQCRICMRNFSDRSNLSRHIRTHTGEKPFACDICGRKFAVAEYRYKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 315 - protein, 298 aa, Artificial Sequence
  source: /note=“Description of Artificial Sequence Synthetic polypeptide”
MAPKKKRKVGVPAAMAERPFQCRICMQNFSQSGNLARHIRTHTGEKPFACDICGRKFAQRIDLTRHTKIHTHPRAPIPKPFQCRICMQNFSRSADLTRHIRTHTGEKPFACDICGRKFAQSGDLTRHTKIHTGSQKPFQCRICMQNFSDRSNLSRHIRTHTGEKPFACDICGRKFAVAEYRYKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 316 - protein, 294 aa, Artificial Sequence
  source: /note=“Description of Artificial Sequence Synthetic polypeptide”
MAPKKKRKVGVPAAMAERPFQCRICMRNFSRSDHLSEHIRTHTGEKPFACDICGRKFAYSWRLSQHTKIHTGSQKPFQCRICMRNFSLRHHLTRHIRTHTGEKPFACDICGRKFAQSGTRKTHTKIHTGSQKPFQCRICMRNFSRSDVLSKHIRTHTGEKPFACDICGRKFARSDDRIKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 317 - protein, 299 aa, Artificial Sequence
  source: /note=“Description of Artificial Sequence Synthetic polypeptide”
MAPKKKRKVGVPAAMAERPFQCRICMQNFSWNSDLRKHIRTHTGEKPFACDICGRKFAQSGNLARHTKIHTGSQKPFQCRICMQNFSQSSDLSRHIRTHTGEKPFACDICGRKFAHRSTRNRHTKIHTHPRAPIPKPFQCRICMQNFSRSDDLSRHIRTHTGEKPFACDICGRKFAQSANRTKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRSE

SEQ ID NO: 318 - protein, 298 aa, Artificial Sequence
  source: /note=“Description of Artificial Sequence Synthetic polypeptide”
MAPKKKRKVGVPAAMAERPFQCRICMQNFSRSAHLSRHIRTHTGEKPFACDICGRKFADRSDLSRHTKIHTGSQKPFQCRICMQNFSQSSNLARHIRTHTGEKPFACDICGRKFAQSSDLRRHTKIHTHPRAPIPKPFQCRICMQNFSDRSHLTRHIRTHTGEKPFACDICGRKFAQSSDLRRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 319 - protein, 298 aa, Artificial Sequence
  source: /note=“Description of Artificial Sequence Synthetic polypeptide”
MAPKKKRKVGVPAAMAERPFQCRICMQNFSQSAHRKNHIRTHTGEKPFACDICGRKFADSSHRTRHTKIHTHPRAPIPKPFQCRICMQNFSDRSHLTRHIRTHTGEKPFACDICGRKFADRSNLTRHTKIHTGSQKPFQCRICMQNFSDRSNLSRHIRTHTGEKPFACDICGRKFAHKWLRNQHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 320 - protein, 302 aa, Artificial Sequence
  source: /note=“Description of Artificial Sequence Synthetic polypeptide”
MAPKKKRKVGVPAAMAERPFQCRICMQNFSDRSHLTRHIRTHTGEKPFACDICGRKFAQSGNLARHTKIHTHPRAPIPKPFQCRICMQNFSDRSDLSRHIRTHTGEKPFACDICGRKFADRSNLTRHTKIHTHPRAPIPKPFQCRICMQNFSQSSDLSRHIRTHTGEKPFACDICGRKFAQSTHRNAHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 321 - protein, 298 aa, Artificial Sequence
  source: /note=“Description of Artificial Sequence Synthetic polypeptide”
MAPKKKRKVGVPAAMAERPFQCRICMRNFSRSDHLSRHIRTHTGEKPFACDICGRKFADRSARNSHTKIHTGSQKPFQCRICMRNFSQQSDLRVHIRTHTGEKPFACDICGRKFARSADLSRHTKIHTHPRAPIPKPFQCRICMRNFSDRSNRTTHIRTHTGEKPFACDICGRKFAHRSSLRRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 322 - protein, 294 aa, Artificial Sequence
  source: /note=“Description of Artificial Sequence Synthetic polypeptide”
MAPKKKRKVGVPAAMAERPFQCRICMQNFSDRSHLTRHIRTHTGEKPFACDICGRKFADRSNLTRHTKIHTGSQKPFQCRICMQNFSDRSNLSRHIRTHTGEKPFACDICGRKFAHKWLRNQHTKIHTGSQKPFQCRICMQNFSRSDNLARHIRTHTGEKPFACDICGRKFADRSVLHRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 323 - protein, 298 aa, Artificial Sequence
  source: /note=“Description of Artificial Sequence Synthetic polypeptide”
MAPKKKRKVGVPAAMAERPFQCRICMQNFSRSDTLSTHIRTHTGEKPFACDICGRKFADRSSRIKHTKIHTGSQKPFQCRICMQNFSRSDNLARHIRTHTGEKPFACDICGRKFAHKSTLSDHTKIHTHPRAPIPKPFQCRICMQNFSRSAHLSRHIRTHTGEKPFACDICGRKFARSDDRKTHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 324 - protein, 298 aa, Artificial Sequence
  source: /note=“Description of Artificial Sequence Synthetic polypeptide”
MAPKKKRKVGVPAAMAERPFQCRICMQNFSQKGTLGEHIRTHTGEKPFACDICGRKFAQSGHLSRHTKIHTHPRAPIPKPFQCRICMQNFSRSDTLSTHIRTHTGEKPFACDICGRKFADRSSRIKHTKIHTGSQKPFQCRICMQNFSRSDNLARHIRTHTGEKPFACDICGRKFAHKSTLSDHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 325 - protein, 294 aa, Artificial Sequence
  source: /note=“Description of Artificial Sequence Synthetic polypeptide”
MAPKKKRKVGVPAAMAERPFQCRICMRNFSDRSNLSRHIRTHTGEKPFACDICGRKFAARWYLDKHTKIHTGSQKPFQCRICMRNFSDRSNLSRHIRTHTGEKPFACDICGRKFADRSHLARHTKIHTGSQKPFQCRICMRNFSYDYGRYTHIRTHTGEKPFACDICGRKFADRSHLARHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 326 - protein, 294 aa, Artificial Sequence
  source: /note=“Description of Artificial Sequence Synthetic polypeptide”
MAPKKKRKVGVPAAMAERPFQCRICMQNFSDRSNLSRHIRTHTGEKPFACDICGRKFALKQHLTRHTKIHTGSQKPFQCRICMQNFSQSGHLARHIRTHTGEKPFACDICGRKFARKWTLQGHTKIHTGSQKPFQCRICMQNFSRWQYLPTHIRTHTGEKPFACDICGRKFADRSALARHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 327 - protein, 294 aa, Artificial Sequence
  source: /note=“Description of Artificial Sequence Synthetic polypeptide”
MAPKKKRKVGVPAAMAERPFQCRICMQNFSQKGTLGEHIRTHTGEKPFACDICGRKFAWRSSLTAHTKIHTGSQKPFQCRICMQNFSDRSNLSRHIRTHTGEKPFACDICGRKFALKQHLTRHTKIHTGSQKPFQCRICMQNFSQSGHLARHIRTHTGEKPFACDICGRKFARKWTLQGHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 328 - protein, 294 aa, Artificial Sequence
  source: /note=“Description of Artificial Sequence Synthetic polypeptide”
MAPKKKRKVGVPAAMAERPFQCRICMRNFSRSDVLSAHIRTHTGEKPFACDICGKKFARNDHRIKHTKIHTGSQKPFQCRICMRNFSRSDNLSEHIRTHTGEKPFACDICGRKFAKRCNLRCHTKIHTGSQKPFQCRICMRNFSRSADLTRHIRTHTGEKPFACDICGRKFADRSALARHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 329 - protein, 294 aa, Artificial Sequence
  source: /note=“Description of Artificial Sequence Synthetic polypeptide”
MAPKKKRKVGVPAAMAERPFQCRICMRNFSRSDNLSEHIRTHTGEKPFACDICGRKFAKRCNLRCHTKIHTGSQKPFQCRICMRNFSRSADLTRHIRTHTGEKPFACDICGRKFADRSALARHTKIHTGSQKPFQCRICMRNFSQSGHLARHIRTHTGEKPFACDICGRKFAQKGTLGEHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 330 - protein, 294 aa, Artificial Sequence
  source: /note=“Description of Artificial Sequence Synthetic polypeptide”
MAPKKKRKVGVPAAMAERPFQCRICMRNFSRSADLTRHIRTHTGEKPFACDICGRKFADRSALARHTKIHTGSQKPFQCRICMRNFSQSGHLARHIRTHTGEKPFACDICGRKFAQKGTLGEHTKIHTGSQKPFQCRICMRNFSIRSTLRDHIRTHTGEKPFACDICGRKFAHRSSLRRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 331 - protein, 294 aa, Artificial Sequence
  source: /note=“Description of Artificial Sequence Synthetic polypeptide”
MAPKKKRKVGVPAAMAERPFQCRICMQNFSRSADLTRHIRTHTGEKPFACDICGRKFADRSALARHTKIHTGSQKPFQCRICMQNFSQSGHLARHIRTHTGEKPFACDICGRKFAQKGTLGEHTKIHTGSQKPFQCRICMQNFSIRSTLRDHIRTHTGEKPFACDICGRKFAHRSSLRRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 332 - protein, 294 aa, Artificial Sequence
  source: /note=“Description of Artificial Sequence Synthetic polypeptide”
MAPKKKRKVGVPAAMAERPFQCRICMQNFSQSGHLARHIRTHTGEKPFACDICGRKFAQKGTLGEHTKIHTGSQKPFQCRICMQNFSIRSTLRDHIRTHTGEKPFACDICGRKFAHRSSLRRHTKIHTGSQKPFQCRICMQNFSRREDLITHIRTHTGEKPFACDICGRKFATSSNLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 333 - protein, 298 aa, Artificial Sequence
  source: /note=“Description of Artificial Sequence Synthetic polypeptide”
MAPKKKRKVGVPAAMAERPFQCRICMQNFSRSDTLSEHIRTHTGEKPFACDICGRKFAHRRSRWGHTKIHTHPRAPIPKPFQCRICMQNFSRSDVLSEHIRTHTGEKPFACDICGRKFARNQHRKTHTKIHTGSQKPFQCRICMQNFSQSGNLARHIRTHTGEKPFACDICGRKFAAKWNLDAHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 334 - protein, 298 aa, Artificial Sequence
  source: /note=“Description of Artificial Sequence Synthetic polypeptide”
MAPKKKRKVGVPAAMAERPFQCRICMQNFSQSGHLARHIRTHTGEKPFACDICGRKFATSGHLSRHTKIHTGSQKPFQCRICMQNFSWRSCRSAHIRTHTGEKPFACDICGRKFADRSNLTRHTKIHTHPRAPIPKPFQCRICMQNFSQSANRTKHIRTHTGEKPFACDICGRKFATSSNLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 335 - protein, 265 aa, Artificial Sequence
  source: /note=“Description of Artificial Sequence Synthetic polypeptide”
MAPKKKRKVGVPAAMAERPFQCRICMRNFSRSDVLSEHIRTHTGEKPFACDICGRKFAKHSTRRVHTKIHTGSQKPFQCRICMRKFARSDHLSRHTKIHTGEKPFQCRICMRNFSDRSNLSRHIRTHTGEKPFACDICGRKFAWRSSLRAHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 336 - protein, 298 aa, Artificial Sequence
  source: /note=“Description of Artificial Sequence Synthetic polypeptide”
MAPKKKRKVGVPAAMAERPFQCRICMQNFSYKCCLSDHIRTHTGEKPFACDICGRKFARSANLTRHTKIHTGSQKPFQCRICMQNFSRSDHLSAHIRTHTGEKPFACDICGRKFADRSALARHTKIHTHPRAPIPKPFQCRICMQNFSIKHDLHRHIRTHTGEKPFACDICGRKFARSANLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 337 - protein, 294 aa, Artificial Sequence
  source: /note=“Description of Artificial Sequence Synthetic polypeptide”
MAPKKKRKVGVPAAMAERPFQCRICMQNFSYPKDLARHIRTHTGEKPFACDICGRKFATSGNLTRHTKIHTGSQKPFQCRICMQNFSTSGSLSRHIRTHTGEKPFACDICGRKFAQSADRTKHTKIHTGSQKPFQCRICMQNFSQSGDLTRHIRTHTGEKPFACDICGRKFAYRWLRNNHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 338 - protein, 298 aa, Artificial Sequence
  source: /note=“Description of Artificial Sequence Synthetic polypeptide”
MAPKKKRKVGVPAAMAERPFQCRICMQNFSRSADLTRHIRTHTGEKPFACDICGRKFAQSGDLTRHTKIHTHPRAPIPKPFQCRICMQNFSRSDHLSRHIRTHTGEKPFACDICGRKFADRSNRKTHTKIHTGSQKPFQCRICMQNFSTSGSLSRHIRTHTGEKPFACDICGRKFAQNANLMKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 339 - protein, 294 aa, Artificial Sequence
  source: /note=“Description of Artificial Sequence Synthetic polypeptide”
MAPKKKRKVGVPAAMAERPFQCRICMRNFSTSGHLSRHIRTHTGEKPFACDICGRKFAQSGHLSRHTKIHTGSQKPFQCRICMRNFSDRSHLTRHIRTHTGEKPFACDICGRKFAQSSDLRRHTKIHTGSQKPFQCRICMRNFSDRSYRNTHIRTHTGEKPFACDICGRKFARRSDLKRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 340 - protein, 294 aa, Artificial Sequence
  source: /note=“Description of Artificial Sequence Synthetic polypeptide”
MAPKKKRKVGVPAAMAERPFQCRICMRNFSQSGNLARHIRTHTGEKPFACDICGRKFARQEHRVAHTKIHTGSQKPFQCRICMRNFSQSGHLARHIRTHTGEKPFACDICGRKFARNGHLLDHTKIHTGSQKPFQCRICMRNFSRSAHLSRHIRTHTGEKPFACDICGRKFAQSSDLRRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 341 - protein, 298 aa, Artificial Sequence
  source: /note=“Description of Artificial Sequence Synthetic polypeptide”
MAPKKKRKVGVPAAMAERPFQCRICMQNFSQSGHLARHIRTHTGEKPFACDICGRKFAQLTHLNSHTKIHTHPRAPIPKPFQCRICMQNFSRSDNLARHIRTHTGEKPFACDICGRKFAMRHHLDDHTKIHTGSQKPFQCRICMQNFSLRHHLTRHIRTHTGEKPFACDICGRKFADRSYRNTHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 342 - protein, 294 aa, Artificial Sequence
  source: /note=“Description of Artificial Sequence Synthetic polypeptide”
MAPKKKRKVGVPAAMAERPFQCRICMQNFSQSSDLSRHIRTHTGEKPFACDICGRKFAQSGHLSRHTKIHTGSQKPFQCRICMQNFSQSSDLSRHIRTHTGEKPFACDICGRKFAQKGHLRKHTKIHTGSQKPFQCRICMQNFSRSDHLSEHIRTHTGEKPFACDICGRKFAQSANRTKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 343 - protein, 294 aa, Artificial Sequence
  source: /note=“Description of Artificial Sequence Synthetic polypeptide”
MAPKKKRKVGVPAAMAERPFQCRICMRNFSRSDALSEHIRTHTGEKPFACDICGRKFARSSDRTKHTKIHTGSQKPFQCRICMRNFSRSANLARHIRTHTGEKPFACDICGRKFATNQNRITHTKIHTGSQKPFQCRICMRNFSRSAHLSRHIRTHTGEKPFACDICGRKFADRSDLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 344 - protein, 294 aa, Artificial Sequence
  source: /note=“Description of Artificial Sequence Synthetic polypeptide”
MAPKKKRKVGVPAAMAERPFQCRICMQNFSQSGNLARHIRTHTGEKPFACDICGRKFARQEHRVAHTKIHTGSQKPFQCRICMQNFSQSGHLARHIRTHTGEKPFACDICGRKFARNGHLLDHTKIHTGSQKPFQCRICMQNFSRSAHLSRHIRTHTGEKPFACDICGRKFAQSSDLRRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 345 - protein, 294 aa, Artificial Sequence
  source: /note=“Description of Artificial Sequence Synthetic polypeptide”
MAPKKKRKVGVPAAMAERPFQCRICMQNFSDRSNLSRHIRTHTGEKPFACDICGRKFALRQDLKRHTKIHTGSQKPFQCRICMQNFSDRSALSRHIRTHTGEKPFACDICGRKFAQSGDLTRHTKIHTGSQKPFQCRICMQNFSRSANLARHIRTHTGEKPFACDICGRKFADSSDRKKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 346 - protein, 298 aa, Artificial Sequence
  source: /note=“Description of Artificial Sequence Synthetic polypeptide”
MAPKKKRKVGVPAAMAERPFQCRICMRNFSDRSDLSRHIRTHTGEKPFACDICGRKFAQSSTRARHTKIHTHPRAPIPKPFQCRICMRNFSQSSDLSRHIRTHTGEKPFACDICGRKFADRSALARHTKIHTGSQKPFQCRICMRNFSIKHDLHRHIRTHTGEKPFACDICGRKFARSANLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 347 - protein, 5 aa, Artificial Sequence
  source: /note=“Description of Artificial Sequence Synthetic peptide”
GGGGS

SEQ ID NO: 348 - protein, 7 aa, Artificial Sequence
  source: /note=“Description of Artificial Sequence Synthetic peptide”
TGEKPFA

SEQ ID NO: 349 - protein, 8 aa, Artificial Sequence
  source: /note=“Description of Artificial Sequence Synthetic peptide”
TGSQKPFQ

SEQ ID NO: 350 - protein, 12 aa, Artificial Sequence
  source: /note=“Description of Artificial Sequence Synthetic peptide”
LRQKDAARGSGG

SEQ ID NO: 351 - protein, 6 aa, Artificial Sequence
  source: /note=“Description of Artificial Sequence Synthetic peptide”
LRGSGG

(19) United States
(12) Patent Application Publication
(10) Pub. No.: US 2023/0002459 A1
(43) Pub. Date: Jan. 5, 2023

(54) Zinc Finger Protein Transcription Factors for Treatment of Prion Disease

(71) Applicant: Sangamo Therapeutics, Inc., Brisbane, CA (US)

(72) Inventors: Bryan Zeitler, El Sobrante, CA (US); Lei Zhang, Davis, CA (US)

(73) Assignee: Sangamo Therapeutics, Inc., Brisbane, CA (US)

(21) Appl. No.: 17/766,083

(22) PCT Filed: Oct. 2, 2020
(86) PCT No.: PCT/US2020/054140

§ 371 Date: Apr. 1, 2022

Related U.S. Application Data
(60) Provisional application No. 62/909,725, filed on Oct. 2, 2019.
(60) Provisional application No. 63/023,197, filed on May 11, 2020.

(51) Int. Cl. C07K 14/47; A61K 48/00; A61P 25/28; C12N 15/86

(52) U.S. Cl. CPC C07K 14/4703; A61K 48/005; A61K 48/0008; A61P 25/28; C12N 15/86; C07K 2319/81; C12N 2750/14143; C12N 2750/14171; A61K 38/00

(57) ABSTRACT

The present disclosure provides zinc finger fusion proteins that inhibit expression of the prion gene in the nervous system, and methods of using the proteins to treat prion disease.

### CROSS REFERENCE TO RELATED APPLICATIONS

The present application claims priority from U.S. Provisional Applications 62/909,725, filed on Oct. 2, 2019, and 63/023,197, filed May 11, 2020. The contents of the aforementioned provisional applications are incorporated herein by reference in their entirety.

### SEQUENCE LISTING

The instant application contains a Sequence Listing which has been submitted electronically in ASCII format and is hereby incorporated by reference in its entirety. Said ASCII copy, created on Oct. 2, 2020, is named 025297_WO012_SL.txt and is 197,128 bytes in size.

### BACKGROUND OF THE INVENTION

Prion disease refers to a group of progressive neurodegenerative disorders that affect both humans and animals. These disorders are characterized by the accumulation of a misfolded isoform of the prion protein (PrP Scrapie; PrP^{Sc}) leading to spongiform changes to the brain associated with neuronal loss and gliosis. The term prion, short for proteinatious infectious particle, refers to the protein-only nature of these pathogenic isoforms. The abnormally shaped protein (PrP^{Sc}) can subsequently bind and convert the abundantly expressed physiological form of the prion protein (cellular PrP; PrP^{C}) to the disease-causing isoform PrP^{Sc}. This phenomenon is known as self-templating. Although identical in protein sequence, PrP^{Sc} is drastically different from PrP^{C} biophysically in terms of solubility, structure, and stability (Riesner, Brit Med Bull. (2003) 66:21-33). Propagation of the PrP^{Sc} isoform is followed by aggregation, causing neuronal cell death in the central nervous system. Human prion diseases can be genetic (accounting for 10-15% of cases), sporadic or acquired and include Creutzfeldt-Jakob Disease (CJD), Gerstmann-Straussler-Scheinker Syndrome (GSS), Fatal Familial Insomnia (FFI), and Kuru. In humans, prion disease impairs brain function, causing progressive cognitive decline and abnormal movements. Prion disease is always fatal, and typically results in death within a few months to several years of onset of illness.

The precise function of PrP is still controversial in the field, but among many phenotypes, PrP has been hypothesized to play a role in neurogenesis and neuroprotection, circadian rhythm, myelin maintenance, epithelial to mesenchymal transition (EMT) and long-term potentiation (LTP). In addition to the familial forms, prion disease can also be sporadic or acquired. People with sporadic prion disease have no family history of the disease or identifiable mutation in the PRNP gene. Sporadic prion disease occurs when PrP^{C} is spontaneously transformed into PrP^{Sc}. Sporadic forms of prion disease include sporadic CJD (sCJD), sporadic fatal insomnia (sFI), and variably protease-sensitive prionopathy (VPSPr). Acquired prion disease results from exposure to PrP^{Sc} from an outside source. For example, variant CJD (vCJD) is a form of acquired prion disease resulting from consumption of beef products containing PrP^{Sc} from cattle with prion disease. In cattle, this form of the disease is known as bovine spongiform encephalopathy (BSE) or “mad cow disease.” Another example of an acquired human prion disease is Kuru, which was identified in the South Fore population in Papua New Guinea. Kuru was transmitted when individuals ate affected human tissue during cannibalistic funeral rituals.

Reduction of PrP expression is a therapeutic strategy supported by numerous genetic proof of concept studies as well as by the in vivo efficacy of PrP-lowering antisense oligonucleotides (ASOs) shown to prolong the survival of prion-infected mice. See, e.g., Büeler et al., Cell (1993) 73(7):1339-47; Büeler et al., Mol Med Camb Mass. (1994) 1(1):19-30; Fischer et al., EMBO J. (1996) 15(6):1255-64; Mallucci et al., Science (2003) 302(5646):871-4; and Safar et al., J Gen Virol. (2005) 86(Pt 10):2913-23, Minikel et al., Nucleic Acids Res. (2020) 10.1093/nar/gkaa616. While ASOs achieving 50% PrP knockdown have been shown to delay the onset of prion disease in mice by more than two-fold, greater levels of PrP knockdown may offer further therapeutic benefit. Distribution of PrP knockdown beyond what is achievable with ASOs may also be important for efficacy. Thus, there remains a need for an effective treatment of prion disease by targeting PrP expression.

### SUMMARY OF THE INVENTION

The present disclosure provides zinc finger protein (ZFP) domains that target sites in or near the mammalian (e.g., human, non-human primate, rodent, or murine) PRNP gene. The ZFP domains of the present disclosure may be fused to a transcription factor to specifically inhibit the mammalian PRNP gene at the DNA level. These fusion proteins, also termed zinc finger protein transcription factors (ZFP-TFs), comprise (i) a ZFP domain that binds specifically to a target region in the PRNP gene and (ii) a transcription repressor domain that reduces the transcription of the gene. These proteins can be used to treat prion disease.

In one aspect, the present disclosure provides a fusion protein comprising a zinc finger protein (ZFP) domain and a transcription repressor domain, wherein the ZFP domain binds to a target region of a mammalian (e.g., human, non-human primate, rodent or murine) prion protein gene (PRNP gene). In some embodiments, the target region of the ZFP-TF is within about 1 kb or 500 bp of a transcription start site (TSS) in the PRNP gene. In some embodiments, the fusion protein may comprise one or more (e.g., two, three, four, five, or six) zinc fingers and it optionally represses expression of the PRNP gene by at least about 40%, 75%, 90%, 95%, or 99% with no or minimal detectable off-target binding or activity. Nonlimiting examples of zinc finger domains are shown in the tables in FIGS. 4 and 8A. In some embodiments, the fusion protein comprises one or more recognition helix sequences shown in the tables in FIGS. 4 and 8A. In further embodiments, the fusion protein comprises some or all the recognition helix sequences from a single row of the tables in FIGS. 4 and 8A, with or without the indicated backbone mutation(s). In certain embodiments, the fusion protein comprises an amino acid sequence shown in the tables in FIG. 9A or 9B.

In some embodiments, the transcription repression domain of the fusion protein may comprise a KRAB domain amino acid sequence of KOX1. In the fusion protein, the ZFP domain may be linked to the transcription repressor domain through a peptide linker.

In another aspect, the present disclosure provides a nucleic acid construct comprising a coding sequence for the present fusion protein, wherein the coding sequence is linked operably to a transcription regulatory element, such as a mammalian promoter that is constitutively active or inducible in a brain cell (e.g., a human synapsin I promoter). The present disclosure also provides a host cell comprising the nucleic acid construct. The host cell may be, e.g., a human cell, and/or a brain cell or a pluripotent stem cell, wherein the stem cell is optionally an embryonic stem cell or an inducible pluripotent stem cell (iPSC). The present disclosure also provides a recombinant virus (recombinant adeno-associated virus (AAV), optionally of serotype 6 or 9) comprising the nucleic acid construct.

In yet another aspect, the present disclosure provides a method of inhibiting expression of prion protein (PrP) in a mammalian brain cell, comprising introducing into the cell the present fusion protein, optionally through introduction of a nucleic acid construct (e.g., through a recombinant virus) described herein, thereby inhibiting the expression of PrP in the cell. The mammalian brain cell may be, for example, a human, non-human primate, rodent, or murine cell, and/or may be a neuron, a glial cell, an ependymal cell, or a neuroepithelial cell. In some embodiments, the cell is in the brain of a patient suffering from or at risk of developing prion disease, wherein the prion disease is optionally familial, sporadic, or acquired prion disease, such as Creutzfeldt-Jakob Disease (CJD), sporadic CJD, variant CJD, Gerstmann-Straussler-Scheinker Syndrome (GSS), Fatal Familial Insomnia (FFI), sporadic Fatal Insomnia (sFI), Kuru, or variably protease-sensitive prionopathy (VPSPr).

The present disclosure also provides a method of treating a neurodegenerative disease in a patient, comprising administering to the patient a recombinant AAV encoding the present fusion protein. The neurodegenerative disease may be a prion disease, wherein the prion disease is optionally familial, sporadic, or acquired prion disease, such as CJD, sporadic CJD, variant CJD, GSS, FFI, sFI, Kuru, or VPSPr. In some embodiments, the disease may be a tauopathy, such as Alzheimer's disease (AD), progressive supranuclear palsy (PSP), frontotemporal dementia (FTD), or corticobasal degeneration (CBD), or chronic traumatic encephalopathy (CTE). In other embodiments, the disease may be a synucleinopathy, such as Parkinson's disease (PD), multiple systems atrophy (MSA), or dementia with Lewy bodies (DLB).

In some embodiments, the AAV encoding the present fusion protein is introduced to the patient via intravenous, intrathecal, intracerebroventrical, intra-cisternal magna, or intrathalamic injection, or injection into any cerebral region.

The present disclosure provides the present fusion proteins, nucleic acid constructs, and recombinant viruses for use in the methods described in, as well as use of the fusion proteins, nucleic acid constructs, recombinant viruses for the manufacture of a medicament in the methods described herein.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE DRAWINGS

FIG. 1 is a diagram illustrating an upstream genomic region of the mouse Prnp gene. The small triangles in the clusters underneath the gene depict the targeted genomic regions of the 384 ZFP-TFs that were generated to target the mouse Prnp gene. Triangles pointing to the right indicate that the ZFP-TFs bind the sense strand of the gene. Triangles pointing to the left indicate that the ZFP-TFs bind the antisense strand of the gene. 192 of the ZFP-TFs were parental proteins having standard architectures. The other 192 proteins were variants, one for each parent, incorporating three R-to-Q substitutions at the 4th position N-terminal of the 1^{st} amino acid of the zinc fingers helix in three of the zinc fingers of the ZFP-TFs.

FIG. 2 is a diagram showing the effect of each of the parental and variant ZFP-TFs on reducing mouse Prnp mRNA expression in Neuro2a cells harvested 24 hours after transfection with ZFP-TF mRNA. Messenger RNA levels were measured by RT-qPCR. Normalized Prnp expression levels are indicated by the gradient bar “PRNP mRNA.” Deepest (red) color denotes 100% reduction. Lightest color (white) denotes 0% reduction. RT-qPCR data were normalized to the mean of mRNA levels of ATP5B and EIF4A2.

FIGS. 3A-D are panels of bar graphs showing the effect of each of the 384 ZFP-TFs (#81007-#81390) on reducing mouse Prnp mRNA expression upon dose titration (at ZFP-TF mRNA doses of 3 ng, 10 ng, 30 ng, 100 ng, 300 ng and 1000 ng, left to right).

FIG. 4 is a table showing the recognition helix sequences and the genomic sequences bound in or near the mouse PRNP gene by 36 selected engineered ZFPs. For each ZFP, the genomic target sequence (Binding Sequence) and the DNA-binding recognition helix sequences (i.e., F1-F6) of each zinc finger within the ZFP domain are shown in a single row. “\hat{ }” in the table indicates that the arginine (R) residue at the 4th position upstream of the 1st amino acid in the indicated helix is changed to glutamine (Q). SEQ ID NOs assigned to each sequence are shown in parenthesis following the sequence.

FIG. 5 is a panel of bar graphs showing the Prnp repressing activity of the 36 selected ZFP-TFs in Neuro2a cells, assessed as indicated above for FIGS. 3-D.

FIGS. 6A and 6B show Prnp mRNA levels in primary mouse cortical neurons seven days after infection with recombinant AAV encoding one of the 36 selected ZFP-TFs. The neurons were infected with increasing AAV multiplicity of infection (MOI) (from left to right: 1E2, 3E2, 1E3, 3E3, 1E4, and 3E4). FIG. 6A is a table showing normalized RT-qPCR data (mean Prnp mRNA levels and standard deviations). The data are then graphed into bar graphs shown in FIG. 6B. RT-qPCR data were normalized to the mean of mRNA levels of ATP5B, EIF4A2, and GAPDH.

FIG. 7A is a panel of volcano plots depicting the off-target activity of the 36 tested mouse Prnp ZFP-TFs in mouse primary neurons. The volcano plots summarize microarray data showing changes in the transcriptome of mouse primary neurons 7 days after AAV6 transduction. In the volcano plots, green circles (right side of each volcano plot) represent off-target genes significantly upregulated (FDR P<0.05), and red circles represent off-target genes significantly downregulated by (FDR P<0.05). Yellow circles indicate the microarray probe set covering both the mouse Prnp and Prnd (which is located downstream of Prnp) loci. Prnd is not substantially expressed in mouse cortical neurons; therefore, minimal changes in Prnp expression were detected.

FIG. 7B is a table showing the number of dysregulated off-target genes for mouse Prnp ZFP-TFs tested in mouse primary cortical neurons corresponding to the volcano plots in FIG. 7A.

FIG. 8A is a table showing the recognition helix sequences and the genomic sequences bound in or near the human PRNP gene by 12 selected engineered ZFPs. For each ZFP, the genomic target sequence (Binding Sequence) and the DNA-binding recognition helix sequences (i.e., F1-F6) of each zinc finger within the ZFP domain are shown in a single row. “\hat{ }” in the table indicates that the arginine (R) residue at the 4th position upstream of the 1st amino acid in the indicated helix is changed to glutamine (Q). SEQ ID NOs assigned to each sequence are shown in parenthesis underneath the sequence.

FIG. 8B is a panel of graphs showing the 12 ZFP-TFs from FIG. 8A targeting human PRNP in human iPSC-derived neurons. The y-axis is PRNP mRNA expression normalized to the geometric mean of three housekeeping genes (ATP5B, EIF4A2, and GAPDH) and assessed 31 days after transduction of iPSC-derived neurons with AAV6 encoding the different ZFP-TFs. The amount of AAV6 used is indicated at the x-axis, with AAV6 doses increasing from left to right (1E3, 3E3, 1E4, 3E4, 1E5, and 3E5). The bars represent the mean of four technical replicates and the error bars represent standard deviation. Enlarged versions of the titration scales are shown at the bottom of the figure.

FIG. 8C is a table showing repression of human PNRP in human iPSC-derived neurons.

FIG. 8D is a panel of volcano plots depicting the off-target activity of the 12 tested human PRNP ZFP-TFs in human iPSC-derived neurons. The volcano plots summarize microarray data showing changes in the transcriptome of human iPSC-derived neurons 19 days after AAV6 transduction. In the volcano plots, green circles (right side of each volcano plot) represent off-target genes significantly unregulated (FDR P<0.05), and red circles represent off-target genes significantly downregulated by (FDR P<0.05). Yellow circles indicate the microarray probe set detecting transcripts expressed from the human PRNP gene.

FIG. 8E is a table showing the number of dysregulated off-target genes for human PRNP ZFP-TFs tested in human iPSC-derived neurons, corresponding to the volcano plots in FIG. 8D.

FIG. 9A is a table showing the full AA sequence (helix, R(−5)Q variant, intramodule & intermodule linker) of the mouse Prnp ZFP-TFs. FIG. 9B is a table showing the full AA sequence (helix, R(−5)Q variant, intramodule & intermodule linker) of the human PRNP ZFP-TFs. SEQ ID NOs assigned to each sequence are shown in parenthesis following the sequence.

### DETAILED DESCRIPTION OF THE INVENTION

The present disclosure provides ZFP domains that target sites (i.e., sequences) in or near the mammalian PRNP gene. A ZFP domain as described herein may be attached or fused to another functional molecule or domain. The ZFP domains of the present disclosure may be fused to a transcription factor to repress the transcription of the mammalian PRNP gene into RNA. The fusion proteins are called zinc finger protein transcription factors (ZFP-TFs). These ZFP-TFs comprise a zinc finger protein (ZFP) domain that binds specifically to a target region (i.e., target site) in or near the PRNP gene and a transcription repressor domain that reduces the transcription of the gene. Reducing the level of PrP in neurons by introducing the ZFP-TFs into the brain of a patient is expected to inhibit (e.g., reduce or stop) the formation and spread of PrP^{Sc}, thereby treating prion disease (e.g., alleviating symptoms, preventing onset or worsening of symptoms, and increasing survival).

Our ZFP-TF approach to PRNP inhibition has several advantages over the current approaches being tested by others. ZFP-TFs can achieve higher levels of PrP repression than what has been reported for antisense oligonucleotides (ASOs). Further, ZFP-TFs may need to be administered only once (by introducing to the patient a ZFP-TF expression construct such as recombinant viruses, e.g., recombinant AAV), while ASOs require repeated dosing. In addition, the ZFP-TF approach only needs to engage the two alleles of the PRNP gene in the genome of each cell. By contrast, ASOs need to engage numerous copies of the PRNP mRNA in each cell. In addition, with the use of recombinant viruses such as recombinant AAV, ZFP-TFs can be delivered to any brain region of interest.

Our approach to prion disease treatment is expected to be safe. About 1:18,000 humans are estimated to be heterozygous for loss-of-function PRNP mutation and yet there are no discernible deleterious effects in these individuals.

### I. Targets of the ZFP Domains

The ZFP domains of the present fusion proteins bind specifically to a target region in or near the mammalian (e.g., human, non-human primate, or murine) PRNP gene. The DNA-binding ZFP domain of the ZFP-TFs directs the fusion proteins to a target region of the PRNP gene and brings the transcription repressor domain of the fusion proteins to the target region. The repressor domain then represses the PRNP gene's transcription by RNA polymerase. The target region can be any suitable site in the PRNP gene that allows repression of gene expression. By way of example, the target region includes, or is adjacent to (either downstream or upstream of) a PRNP transcription start site (TSS), or a PRNP transcription regulatory element (e.g., promoter, enhancer, RNA polymerase pause site, and the like). For example, the target region may be within about 500-1,000 bp upstream and/or downstream of the TSS.

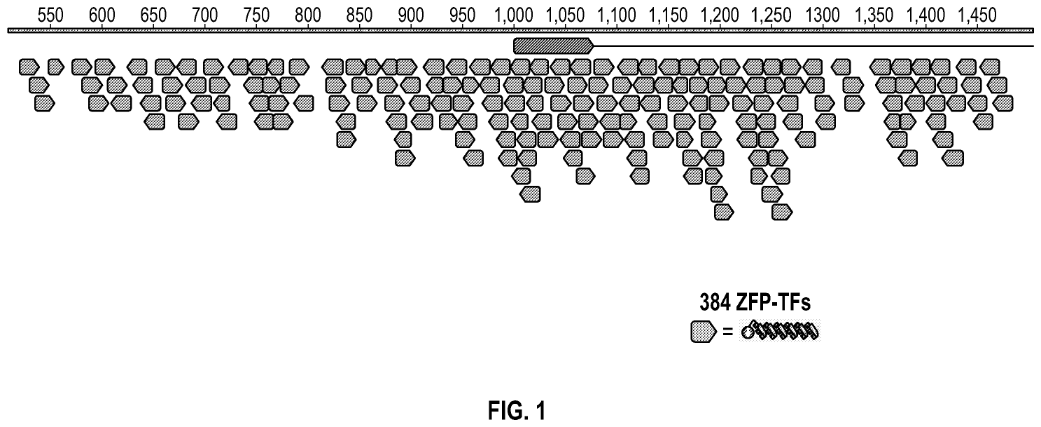

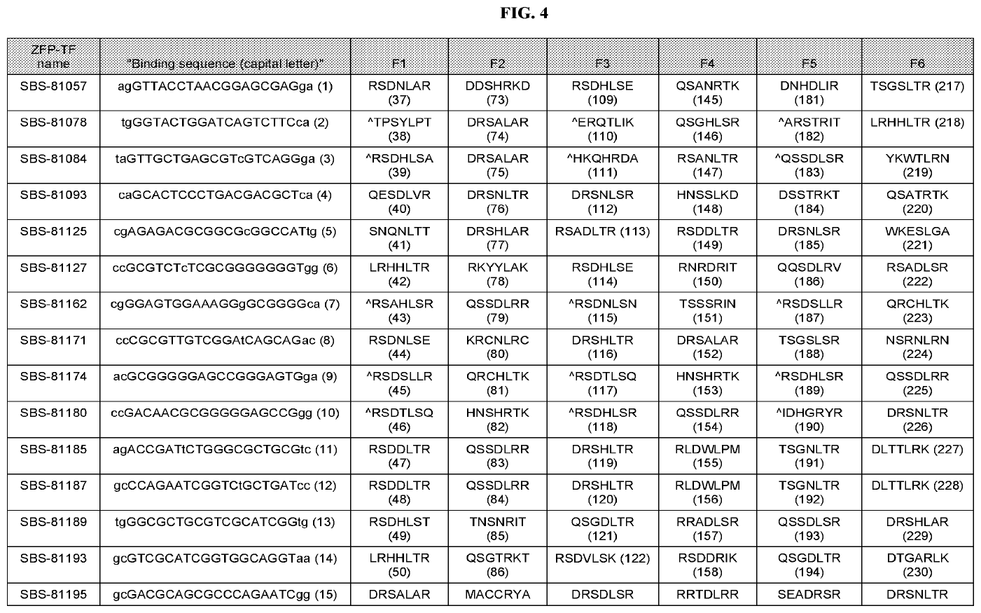

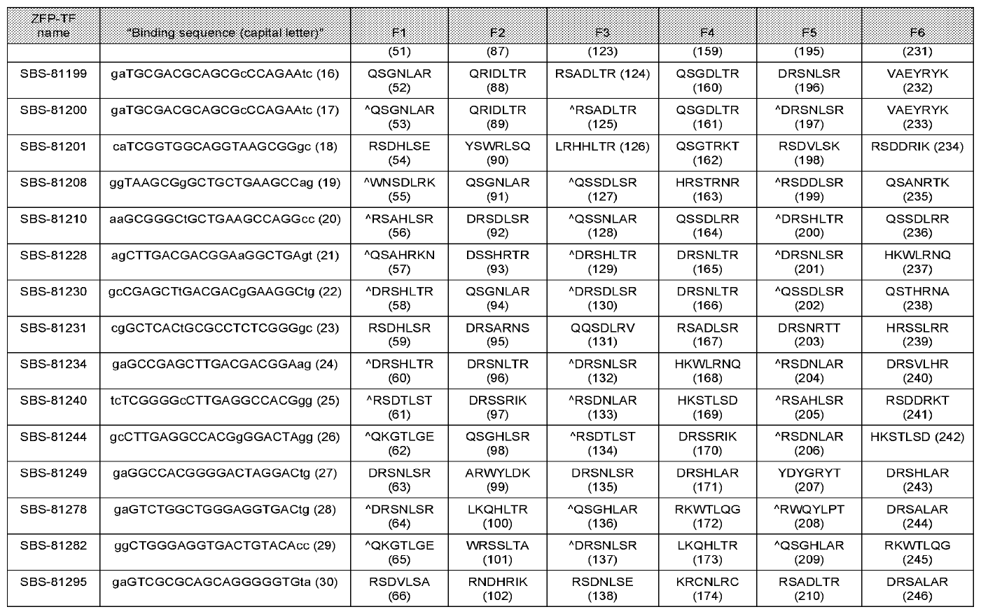

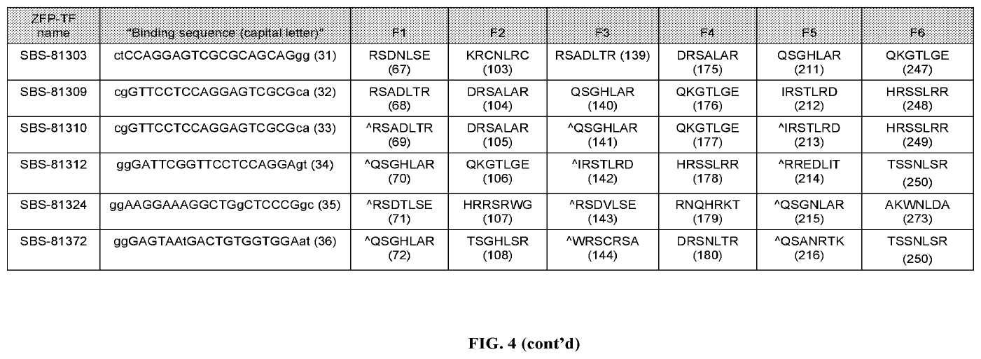

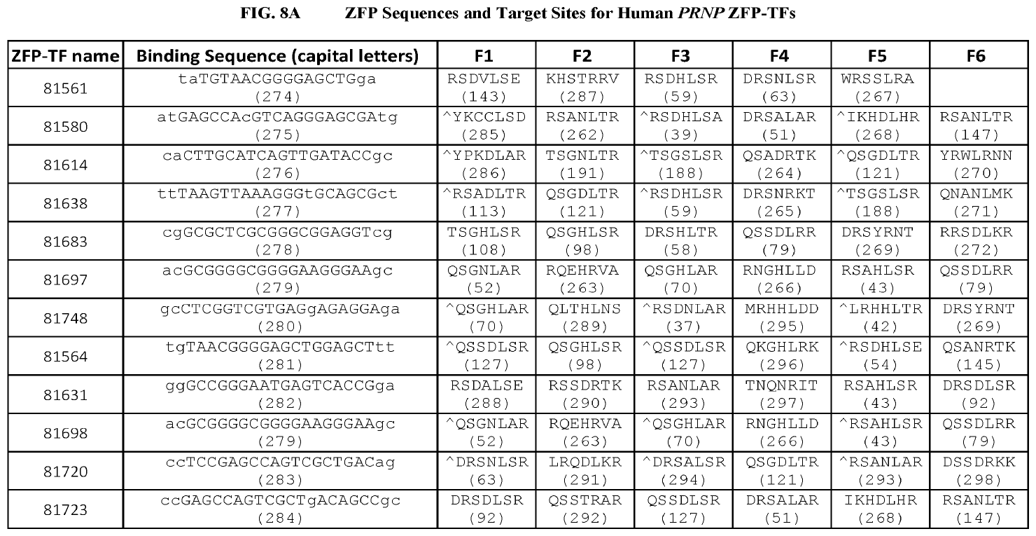

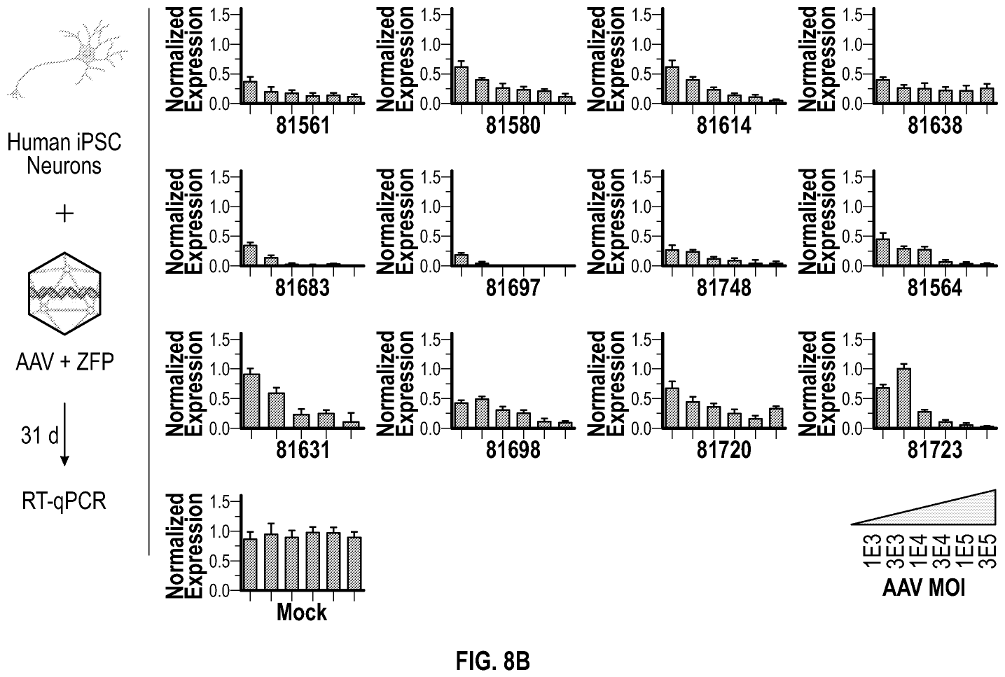

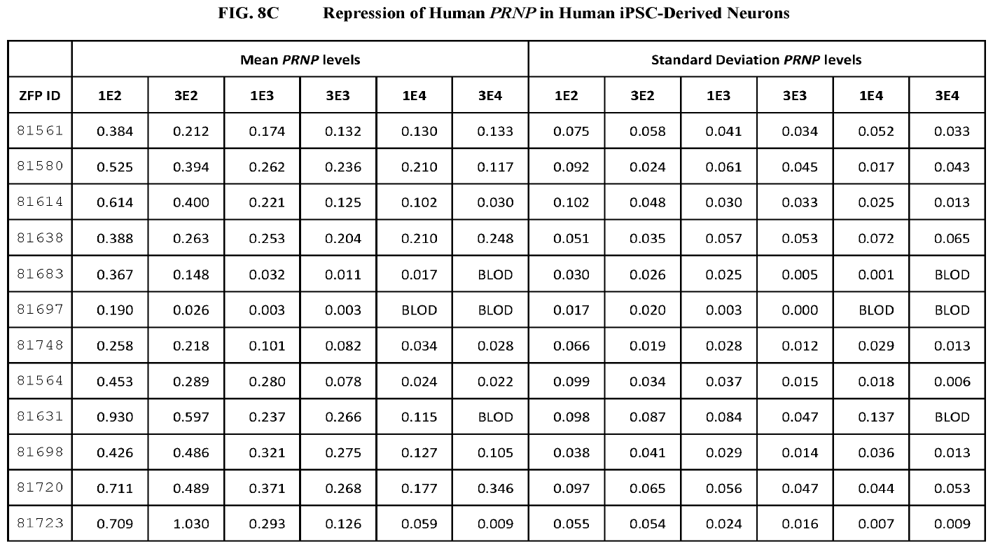

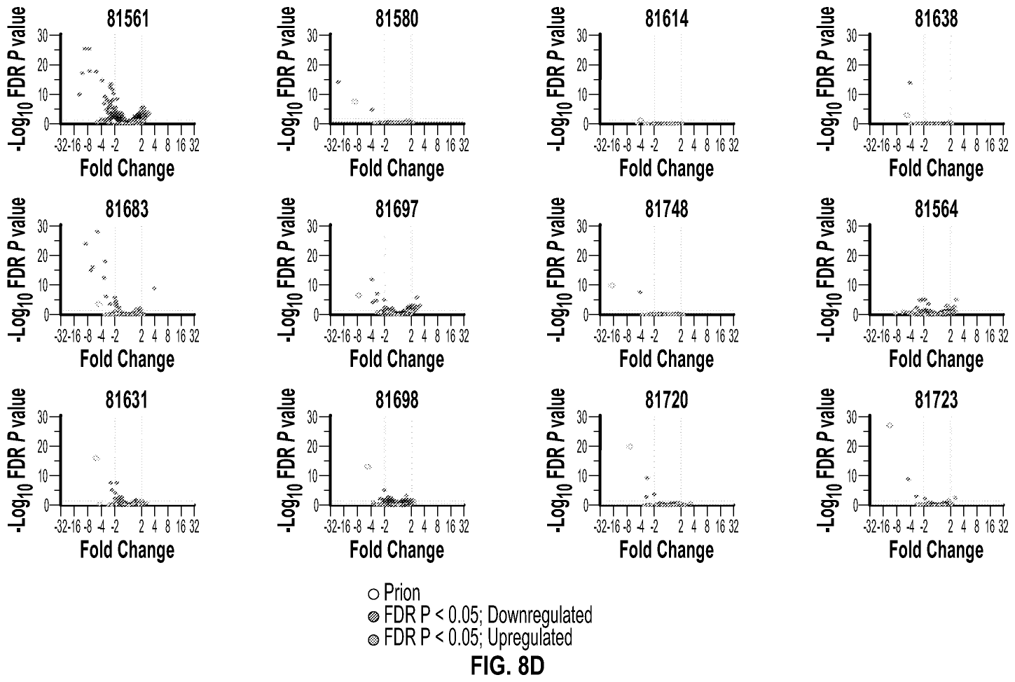

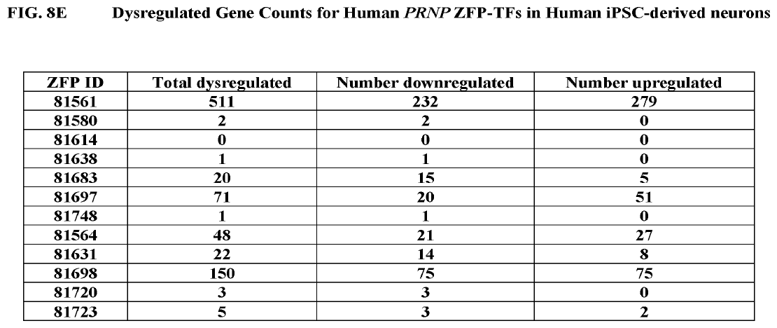

In some embodiments, the genomic target region is at least 8 bps in length. For example, the target region may be 8 bps to 40 bps in length, such as 12, 15, 18, 21, 24, 27, 30, 33, or 36 bps in length. The targeted sequence may be on the sense strand of the gene, or the antisense strand of the gene. To ensure targeting accuracy and to reduce off-target binding or activity by the ZFP-TFs, the sequence of the selected PRNP target region preferably has less than 75% homology (e.g., less than 70%, less than 65%, less than 60%, or less than 50%) to sequences in other genes. In certain embodiments, the target region of the present ZFP-TFs is 15-18 bps in length and resides within bout 500-1,000 bps of the TSS. Examples of target regions in the mouse PRNP gene are shown in FIGS. 1 and 4. Examples of target regions in the human PRNP gene are shown in FIG. 8A.

In some embodiments, the present engineered ZFPs bind to a target site (i.e., Binding Sequence) as shown in a single row of the tables in FIGS. 4 and 8A, preferably with no or little detectable off-target binding or activity.

Other criteria for further evaluating target segments include the prior availability of ZFPs binding to such segments or related segments, ease of designing new ZFPs to bind a given target segment, and off-target binding assessment.

### II. ZFP Domains

A “zinc finger protein” or “ZFP” refers to a protein having a DNA-binding domain that is stabilized by zinc. ZFPs bind to DNA in a sequence-specific manner. The individual DNA-binding unit of a ZFP is referred to as a zinc “finger”. Each finger contains a DNA-binding “recognition helix” that is typically comprised of seven amino acid residues and determines DNA binding specificity. A ZFP domain has at least one finger, each finger binds from two to four base pairs of DNA, typically three or four base pairs of DNA. Each zinc finger typically comprises approximately 30 amino acids and chelates zinc. An engineered ZFP can have a novel binding specificity, compared to a naturally-occurring ZFP. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers that bind the particular triplet or quadruplet sequence. See, e.g., ZFP design methods described in detail in U.S. Pat. Nos. 5,789,538; 5,925,523; 6,007,988; 6,013,453; 6,140,081; 6,200,759; 6,453,242; 6,534,261; 6,979,539; and 8,586,526; and International Patent Publications WO 95/19431; WO 96/06166; WO 98/53057; WO 98/53058; WO 98/53059; WO 98/53060; WO 98/54311; WO 00/27878; WO 01/60970; WO 01/88197; WO 02/016536; WO 02/099084; and WO 03/016496. A ZFP domain as described herein may be attached or fused to another molecule, for example, a protein. Such ZFP-fusions may comprise a domain that enables gene activation (e.g., activation domain), gene repression (e.g., repression domain), ligand binding (e.g., ligand-binding domain), high-throughput screening (e.g., ligand-binding domain), localized hypermutation (e.g., activation-induced cytidine deaminase domain), chromatin modification (e.g., histone deacetylase domain), recombination (e.g., recombinase domain), targeted integration (e.g., integrase domain), DNA modification (e.g., DNA methyl-transferase domain), base editing (e.g., base editor domain), or targeted DNA cleavage (e.g., nuclease domain). Examples of engineered ZFP domains are shown in the tables in FIGS. 4 and 8A.

The ZFP domain of the present engineered ZFP fusion proteins may include at least one (e.g., one, two, three, four, five, six, seven, eight, nine, ten, eleven, twelve, thirteen, or more) zinc finger(s). A ZFP domain having one finger typically recognizes a target site that includes 3 or 4 nucleotides. A ZFP domain having two fingers typically recognizes a target site that includes 6 or 8 nucleotides. A ZFP domain having three fingers typically recognizes a target site that includes 9 or 12 nucleotides. A ZFP domain having four fingers typically recognizes a target site that includes 12 to 15 nucleotides. A ZFP domain having five fingers typically recognizes a target site that includes 15 to 18 nucleotides. A ZFP domain having six fingers can recognize target sites that include 18 to 21 nucleotides.

In some embodiments, the present engineered ZFPs comprise a DNA-binding recognition helix sequence shown in the tables in FIGS. 4 and 8A. For example, an engineered ZFP may comprise the sequence of F1, F2, F3, F4, F5, or F6 as shown in the tables in FIGS. 4 and 8A.

In some embodiments, the present engineered ZFPs comprise two adjacent DNA-binding recognition helix sequences shown in a single row of the tables in FIGS. 4 and 8A. For example, an engineered ZFP may comprise the sequences of F1-F2, F2-F3, F3-F4, F4-F5, or F5-F6 as shown in a single row of the tables in FIGS. 4 and 8A.

In some embodiments, the present engineered ZFPs comprise the DNA-binding recognition helix sequences shown in a single row of the tables in FIGS. 4 and 8A. For example, an engineered ZFP may comprise the sequences of F1, F2, F3, F4, F5, and F6 (e.g., F1-F6) as shown in a single row of the tables in FIGS. 4 and 8A.

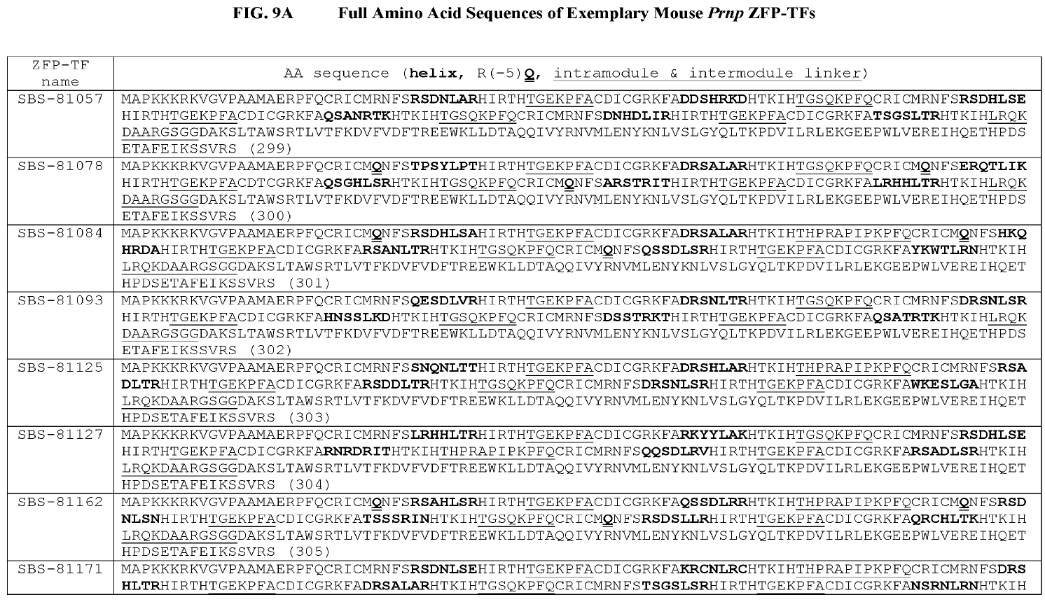

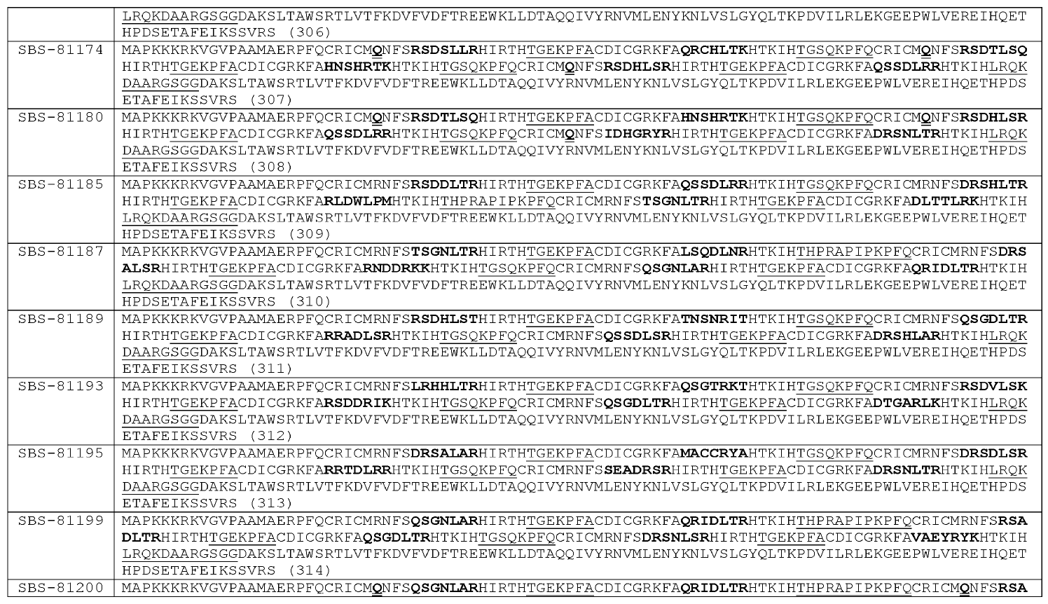

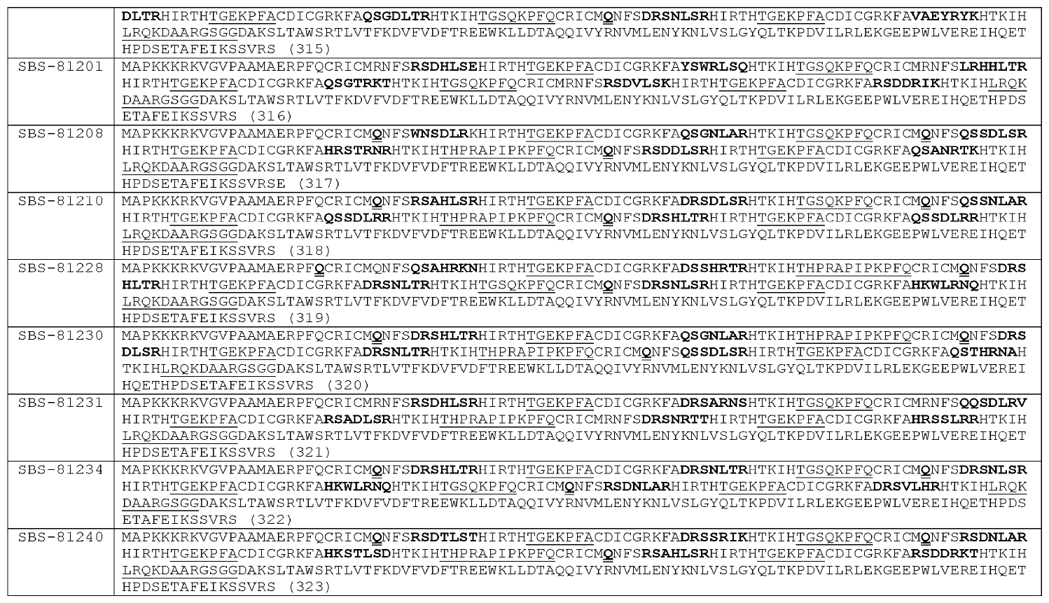

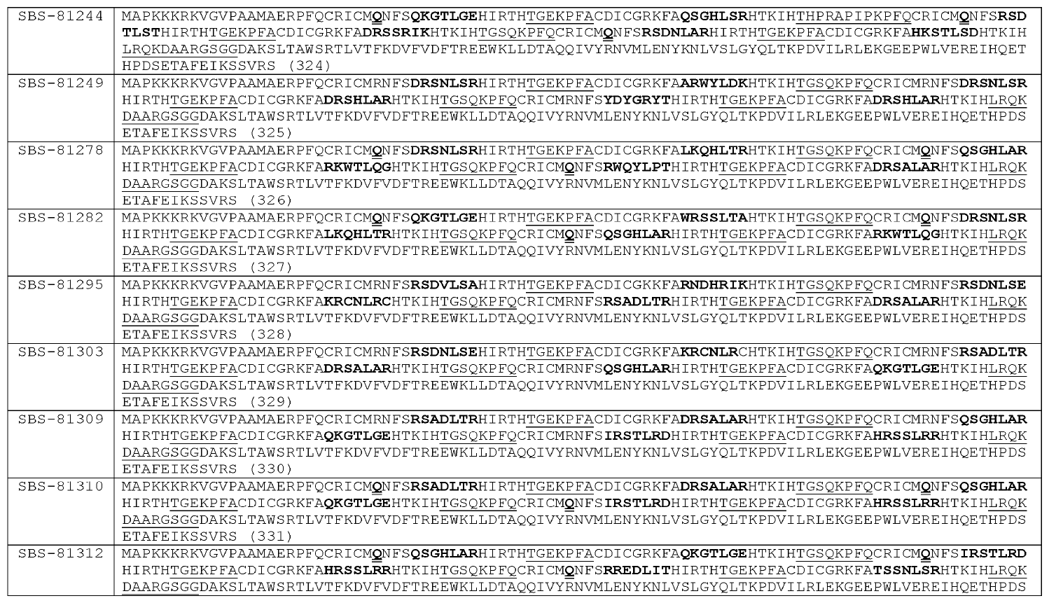

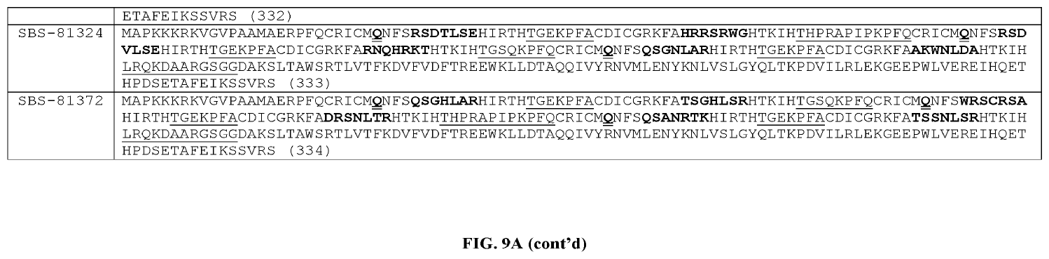

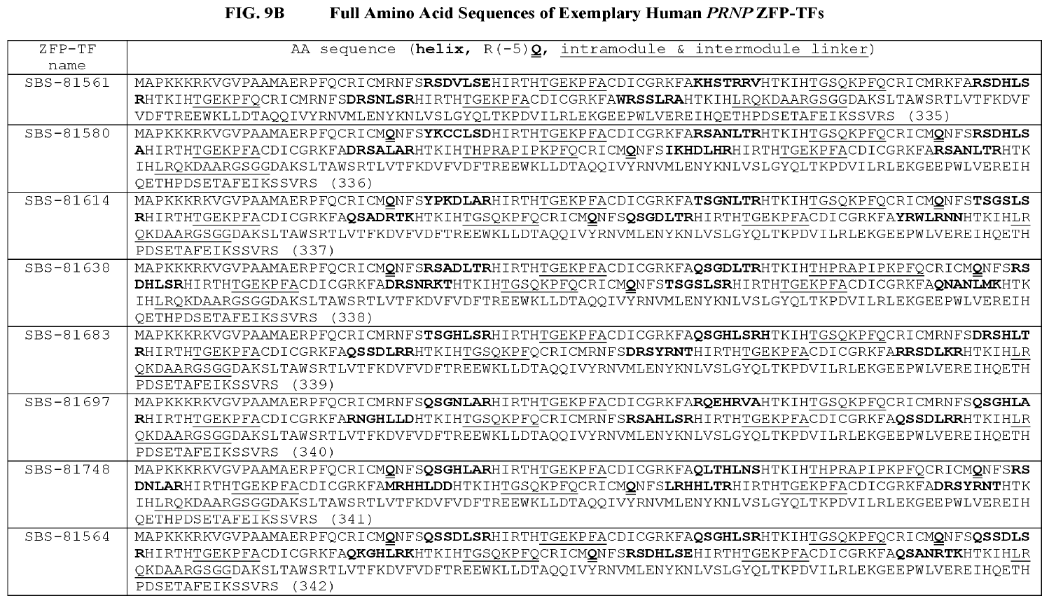

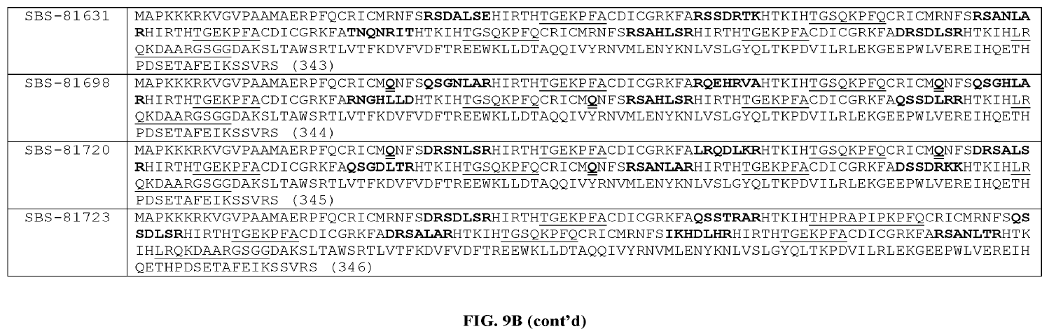

The target specificity of the ZFP domain may be improved by mutations to the ZFP backbone sequence as described in, e.g., U.S. Pat. Pub. 2018/0087072. The mutations include those made to residues in the ZFP backbone that can interact non-specifically with phosphates on the DNA backbone but are not involved in nucleotide target specificity (see, e.g., Miller et al., Nat Biotechnol. (2019) 37(8):945-52). In some embodiments, these mutations comprise mutating a cationic amino acid residue to a neutral or anionic amino acid residue. In some embodiments, these mutations comprise mutating a polar amino acid residue to a neutral or non-polar amino acid residue. In further embodiments, mutations are made at positions (−5), (−9) and/or (−14) relative to the DNA binding helix. In some embodiments, a zinc finger may comprise one or more mutations at positions (−5), (−9) and/or (−14). In further embodiments, one or more zinc fingers in a multi-finger ZFP domain may comprise mutations at positions (−5), (−9) and/or (−14). In some embodiments, the amino acids at positions (−5), (−9) and/or (−14) (e.g., an arginine (R) or lysine (K)) are mutated to an alanine (A), leucine (L), serine (S), aspartate (N), glutamate (E), tyrosine (Y), and/or glutamine (Q). Examples of engineered ZFPs with backbone mutations are shown in the tables in FIGS. 4, 8A, 9A, and 9B. The symbol “\hat{ }” in the tables in FIGS. 4 and 8A indicates that arginine (R) residue at the 4th position upstream of the 1st amino acid in the indicated recognition helix is changed to glutamine (Q), which is shown in bold in the tables in FIGS. 9A and 9B. In each recognition helix sequence, the positions of the seven DNA-binding amino acids are numbered −1, +1, +2, +3, +4, +5, and +6. Thus, the position for the R-to-Q substitution is numbered as (−5).

In some embodiments, the present engineered ZFPs comprise a DNA-binding recognition helix sequence and associated backbone mutation as shown in the tables in FIGS. 4 and 8A. In some embodiments, the present engineered ZFPs comprise the DNA-binding recognition helix sequences and associated backbone mutations as shown in a single row of the tables in FIGS. 4 and 8A.

In some embodiments, an engineered ZFP described herein comprises the recognition helix and backbone portions of a sequence shown in a single row of the tables in FIGS. 9A and 9B. In some embodiments, an engineered ZFP described herein comprises the recognition helix and backbone portions of a sequence shown in a single row of the tables in FIGS. 9A and 9B as the sequence would appear following post-translational modification. For example, post-translational modification may remove the initiator methionine residue from a sequence as shown in a single row of the tables in FIGS. 9A and 9B.

In some embodiments, the present ZFP-TFs comprise one or more zinc finger domains. The domains may be linked together via an extendable flexible linker such that, for example, one domain comprises one or more (e.g., 4, 5, or 6) zinc fingers and another domain comprises additional one or more (e.g., 4, 5, or 6) zinc fingers. In some embodiments, the linker is a standard inter-finger linker such that the finger array comprises one DNA binding domain comprising 8, 9, 10, 11 or 12 or more fingers. In other embodiments, the linker is an atypical linker such as a flexible linker. For example, two ZFP domains may be linked to a transcription repressor TF in the configuration (from N terminus to C terminus) ZFP-ZFP-TF, TF-ZFP-ZFP, ZFP-TF-ZFP, or ZFP-TF-ZFP-TF (two ZFP-TF fusion proteins are fused together via a linker).

In some embodiments, the ZFP-TFs are “two-handed,” i.e., they contain two zinc finger clusters (two ZFP domains) separated by intervening amino acids so that the two ZFP domains bind to two discontinuous target sites. An example of a two-handed type of zinc finger binding protein is SIP1, where a cluster of four zinc fingers is located at the amino terminus of the protein and a cluster of three fingers is located at the carboxyl terminus (see Remade et al., EMBO J. (1999) 18(18):5073-84). Each cluster of zinc fingers in these proteins is able to bind to a unique target sequence and the spacing between the two target sequences can comprise many nucleotides.

Alternatively, the DNA-binding domain may be derived from a nuclease. For example, the recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII are known. See also U.S. Pat. Nos. 5,420,032 and 6,833,252; Belfort et al., Nucleic Acids Res. (1997) 25:3379-88; Dujon et al., Gene (1989) 82:115-8; Perler et al., Nucleic Acids Res. (1994) 22:1125-7; Jasin, Trends Genet. (1996) 12:224-8; Gimble et al., J Mol Biol. (1996) 263:163-80; Argast et al., J Mol Biol. (1998) 280:345-53; and the New England Biolabs catalogue. In addition, the DNA-binding specificity of homing endonucleases and meganucleases can be engineered to bind non-natural target sites. See, for example, Chevalier et al., Mol Cell (2002) 10:895−905; Epinat et al., Nucleic Acids Res. (2003) 31:2952-62; Ashworth et al., Nature (2006) 441:656−59; Paques et al., Current Gene Therapy (2007) 7:49-66; and U.S. Pat. Pub. 2007/0117128.

### III. Zinc-Finger Protein Transcription Factors

The ZFP domains described herein may be fused to a transcription factor. In some embodiments, the transcription factor may be a transcription repressor domain, wherein the ZFP and repressor domains may be associated with each other by a direct peptidyl linkage or a peptide linker, or by dimerization (e.g., through a leucine zipper, a STAT protein N terminal domain, or an FK506 binding protein). As used herein, a “fusion protein” refers to a polypeptide with covalently linked domains as well as a complex of polypeptides associated with each other through non-covalent bonds. The transcription repressor domain can be associated with the ZFP domain at any suitable position, including the C- or N-terminus of the ZFP domain.

In some embodiments, two or more of the present ZFP-TFs are used concurrently in a patient, where the ZFP-TFs bind to different target regions in the PRNP gene, so as to achieve optimal repression of PRNP expression.

### A. Transcription Repressor Domains

The present ZFP-TFs comprise an engineered ZFP domain as described herein and one or more transcription repressor domains that dampen the transcription activity of the PRNP gene. One or more engineered ZFP domains and one or more transcription repressor domains may be joined by a flexible linker. Non-limiting examples of transcription repressor domains are the KNOX1 KRAB domain, KAP-1, MAD, FKHR, EGR-1, ERD, SID, TGF-beta-inducible early gene (TIEG), v-ERB-A, MBD2, MBD3, TRa, histone methyltransferase, histone deacetylase (HDAC), nuclear hormone receptor (e.g., estrogen receptor or thyroid hormone receptor), members of the DNMT family (e.g., DNMT1, DNMT3A, DNMT3B), Rb, and MeCP2. See, e.g., Bird et al. (1999) Cell 99:451-454; Tyler et al. (1999) Cell 99:443-446; Knoepfler et al. (1999) Cell 99:447-450; and Robertson et al. (2000) Nature Genet. 25:338-342. Additional exemplary repression domains include, but are not limited to, ROM2 and AtHD2A. See, for example, Chem et al. (1996) Plant Cell 8:305-321; and Wu et al. (2000) Plant J. 22:19-27.

In some embodiments, the transcription repressor domain comprises a sequence from the Kruppel-associated box (KRAB) domain of the human zinc finger protein 10/KOX1 (ZNF10/KOX1) (e.g., GenBank No. NM 015394.4). An exemplary KRAB domain sequence is:

(SEQ ID NO: 261)
DAKSLTAWSR TLVTFKDVFV DFTREEWKLL DTAQQIVYRN
VMLENYKNLV SLGYQLTKPD VILRLEKGEE PWLVEREIHQ
ETHPDSETAF EIKSSV.

Variants of this KRAB sequence may also be used so long as they have the same or similar transcription repressor function.

In some embodiments, an engineered ZFP-TF described herein binds to a target site as shown in a single row of in the tables in FIGS. 4 and 8A, preferably with no or little detectable off-target binding or activity. Off-target binding may be determined, for example, by measuring the activity of ZFP-TFs at off-target genes.

In some embodiments, an engineered ZFP-TF described herein comprises a DNA-binding recognition helix sequence shown in the tables in FIGS. 4 and 8A. In some embodiments, an engineered ZFP-TF described herein comprises two adjacent DNA-binding recognition helix sequences shown in a single row in the tables in FIGS. 4 and 8A. In some embodiments, an engineered ZFP-TF described herein comprises the DNA-binding recognition helix sequences shown in a single row in the tables in FIGS. 4 and 8A. In some embodiments, an engineered ZFP-TF described herein comprises the recognition helix and backbone portions of a sequence shown in a single row in the tables in FIGS. 9A and 9B. In some embodiments, an engineered ZFP-TF described herein comprises an amino acid sequence as shown in a single row in the tables in FIGS. 9A and 9B.

In some embodiments, an engineered ZFP-TF described herein comprises the recognition helix and backbone portions of a sequence shown in a single row in the tables in FIGS. 9A and 9B as the sequence would appear following post-translational modification. In some embodiments, an engineered ZFP-TF described herein comprises an amino acid sequence as shown in a single row in the tables in FIGS. 9A and 9B as the sequence would appear following post-translational modification. For example, post-translational modification may remove the initiator methionine residue from a sequence as shown in a single row in the tables in FIGS. 9A and 9B.

### B. Peptide Linkers

The ZFP domain and the transcription repressor domain of the present ZFP-TFs and/or the zinc fingers within the ZFP domains may be linked through a peptide linker, e.g., a noncleavable peptide linker of about 5 to 200 amino acids (e.g., 5, 6, 7, 8, 9, 10, 11, 12, 13, 14, 15, 16, 17, 18, 19, 20 or more amino acids). Preferred linkers are typically flexible amino acid subsequences that are synthesized as a recombinant fusion protein. See, e.g., description above; and U.S. Pat. Nos. 6,479,626; 6,903,185; 7,153,949; 8,772,453; and 9,163,245; and WO 2011/139349. The proteins described herein may include any combination of suitable linkers. Non-limiting examples of linkers are DGGGS (SEQ ID NO: 252), TGEKP (SEQ ID NO: 253), LRQKDGERP (SEQ ID NO: 254), GGRR (SEQ ID NO: 255), GGRRGGGS (SEQ ID NO: 256), LRQRDGERP (SEQ ID NO: 257), LRQKDGGGSERP (SEQ ID NO: 258), LRQKD(G₃S)₂ ERP (SEQ ID NO: 259), and TGSQKP (SEQ ID NO: 260). In some embodiments, TGEKPFA (SEQ ID NO: 348) and/or TGSQKPFQ (SEQ ID NO: 349) links the zinc fingers within the ZFP domain, and LRQKDAARGSGG (SEQ ID NO: 350) or LRGSGG (SEQ ID NO: 351) links the ZFP domain to the transcription repressor domain.

In some embodiments, the peptide linker is three to 20 amino acid residues in length and is rich in G and/or S. Non-limiting examples of such linkers are G₄S-type (SEQ ID NO: 347) linkers, i.e., linkers containing one or more (e.g., 2, 3, or 4) GGGGS (SEQ ID NO: 251) motifs, or variations of the motif (such as ones that have one, two, or three amino acid insertions, deletions, and substitutions from the motif).

### IV. Expression of the ZFP-TFs

A ZFP-TF of the present disclosure may be introduced to a patient through a nucleic acid molecule encoding it. The nucleic acid molecule may be an RNA or cDNA molecule. The nucleic acid molecule may be introduced into the brain of the patient through injection of a composition comprising a lipid:nucleic acid complex (e.g., a liposome). Alternatively, the ZFP-TF may be introduced to the patient through a nucleic acid expression vector comprising a sequence encoding the ZFP-TF. The expression vectors may include expression control sequences such as promoters, enhancers, transcription signal sequences, and transcription termination sequences that allow expression of the coding sequence for the ZFP-TFs in the cells of the nervous system. In some embodiments, the expression vector remains present in the cell as a stable episome. In other embodiments, the expression vector is integrated into the genome of the cell.

In some embodiments, the promoter on the vector for directing the ZFP-TF expression in the brain is a constitutive active promoter or an inducible promoter. Suitable promoters include, without limitation, a retroviral RSV LTR promoter (optionally with an RSV enhancer), a CMV promoter (optionally with a CMV enhancer), a CMV immediate early promoter, an SV40 promoter, a dihydrofolate reductase (DHFR) promoter, a β-actin promoter, a phosphoglycerate kinase (PGK) promoter, an EFlα promoter, a MoMLV LTR, a CK6 promoter, a TK promoter, a tetracycline responsive promoter (TRE), an HBV promoter, chimeric liver-specific promoters (LSPs), an E2F promoter, the telomerase (hTERT) promoter, a CMV enhancer/chicken β-actin/rabbit β-globin promoter (CAG promoter; Niwa et al., Gene (1991) 108(2):193−9), and an RU-486-responsive promoter. Neuron- or glial-specific promoters such as a synapsin I promoter, a CAMKII promoter, a MeCP2 promoter, a PrP promoter, a GFAP promoter, or an engineered or natural promoter that restricts expression to neuron and glial cells may also be used.

Any method of introducing the nucleotide sequence into a cell may be employed, including but not limited to, electroporation, calcium phosphate precipitation, microinjection, cationic or anionic liposomes, liposomes in combination with a nuclear localization signal, naturally occurring liposomes (e.g., exosomes), or viral transduction.

For in vivo delivery of an expression vector, viral transduction may be used. A variety of viral vectors known in the art may be adapted by one of skill in the art for use in the present disclosure, for example, vaccinia vectors, adenoviral vectors, lentiviral vectors, poxyviral vectors, adeno-associated viral (AAV) vectors, retroviral vectors, and hybrid viral vectors. In some embodiments, the viral vector used herein is a recombinant AAV (rAAV) vector. AAV vectors are especially suitable for CNS gene delivery because they infect both dividing and non-dividing cells, exist as stable episomal structures for long term expression, and have very low immunogenicity (Hadaczek et al., Mol Ther. (2010) 18:1458-61; Zaiss, et al., Gene Ther. (2008) 15:808-16). Any suitable AAV serotype may be used. For example, the AAV may be AAV1, AAV2, AAV3, AAV3B, AAV4, AAV5, AAV6, AAV7, AAV8, AAV8.2, AAV9, or AAVrh10, or of a pseudotype such as AAV2/8, AAV2/5, AAV2/6 or AAV2/9 (i.e., AAV derived from multiple serotypes; for example, the rAAV comprises AAV2 inverted terminal repeats (ITR) in its genome and an AAV8, 5, 6, or 9 capsid). In some embodiments, the expression vector is an AAV viral vector and is introduced to the target human cell by a recombinant AAV virion whose genome comprises the construct, including having the AAV Inverted Terminal Repeat (ITR) sequences on both ends to allow the production of the AAV virion in a production system such as an insect cell/baculovirus production system or a mammalian cell production system). The AAV may be engineered such that its capsid proteins have reduced immunogenicity or enhanced transduction ability in humans or nonhuman primates. In some embodiments, AAV9 is used. Viral vectors described herein may be produced using methods known in the art. Any suitable permissive or packaging cells may be employed to produce the viral particles. For example, mammalian or insect cells may be used as the packaging cell line.

### V. Pharmaceutical Applications

The present ZFP-TFs can be used to treat patients in need of downregulation of PrP expression. The patients suffer from, or are at risk of developing, prion disease. The prion disease to be treated may be familial, sporadic, or acquired prion disease, and may be CJD, sCJD, vCJD, GSS, FFI, sFI, Kuru, VPSPr. Patients at risk include those who are genetically predisposed and those who have been exposed to meat from cattle with mad cow disease or other environmental sources of prions. The present disclosure provides a method of treating a neurodegenerative disease in a subject such as a human patient in need thereof, comprising introducing to the nervous system of the subject a therapeutically effective amount (e.g., an amount that allows sufficient repression of PRNP expression) of the ZFP-TF (e.g., a rAAV vector expressing it). In some embodiments, the neurodegenerative disease is prion disease. The term “treating” encompasses alleviation of symptoms, prevention of onset of symptoms, slowing of disease progression, and increased survival. Biomarkers including, without limitation, prion or neurofilament light chain (NfL) levels in the cerebrospinal fluid or plasma may also be measured to monitor progress of the treatment.

The present disclosure provides a pharmaceutical composition comprising a viral vector such as a recombinant rAAV whose recombinant genome comprises an expression cassette for the ZFP-TFs. The pharmaceutical composition may further comprise a pharmaceutically acceptable carrier such as water, saline (e.g., phosphate-buffered saline), dextrose, glycerol, sucrose, lactose, gelatin, dextran, albumin, or pectin. In addition, the composition may contain auxiliary substances, such as, wetting or emulsifying agents, pH-buffering agents, stabilizing agents, or other reagents that enhance the effectiveness of the pharmaceutical composition. The pharmaceutical composition may contain delivery vehicles such as liposomes, nanocapsules, microparticles, microspheres, lipid particles, and vesicles.

The cells targeted by the therapeutics of the present disclosure are cells in the brain, including, without limitation, a neuronal cell (e.g., a motor neuron, a sensory neuron, a dopaminergic neuron, a cholinergic neuron, a glutamatergic neuron, a GABAergic neuron, or a serotonergic neuron); a glial cell (e.g., an oligodendrocyte, an astrocyte, a pericyte, a Schwann cell, or a microglial cell); an ependymal cell; or a neuroepithelial cell. The brain regions targeted by the therapeutics may be, for example, cerebral cortex (classic CJD), thalamus (FFI), brain stem (scrapie, BSE, and Chronic Wasting Disease), and cerebellum (Kuru). The targeted brain regions can be reached directly through intrastriatal injection, intrathalamic injection, intracerebral injection, intra-cisterna magna (ICM) injection, or more generally through intraparenchymal injection, intracerebroventricular (ICV) injection, intrathecal injection, or intravenous injection. Other routes of administration include, without limitation, intracerebral, intraventricular, intranasal, or intraocular administration. In some embodiments, the viral vector spreads throughout the CNS tissue following direct administration into the cerebrospinal fluid (CSF), e.g., via intrathecal and/or intracerebral injection, or intracisterna-magna injection. In other embodiments, the viral vectors cross the blood-brain-barrier and achieve wide-spread distribution throughout the CNS tissue of a subject following intravenous administration. In other embodiments, the viral vectors are delivered directly to the target regions via intraparenchymal injections. In some cases, the viral vectors may undergo retrograde or anterograde transport to other brain regions following intraparenchymal delivery. In some aspects, the viral vectors have distinct CNS tissue targeting capabilities (e.g., CNS tissue tropisms), which achieve stable and nontoxic gene transfer at high efficiencies.

By way of example, the pharmaceutical composition may be provided to the patient through intraventricular administration, e.g., into a ventricular region of the forebrain of the patient such as the right lateral ventricle, the left lateral ventricle, the third ventricle, or the fourth ventricle. The pharmaceutical composition may be provided to the patient through intracerebral administration, e.g., injection of the composition into or near the striatum, caudate, putamen, substantia nigra, midbrain, olfactory bulb, cerebrum, medullar, pons, cerebellum, locus coeruleus, pons, medulla, brainstem, globus pallidus, hippocampus, cerebral cortex, cerebrum, intracranial cavity, meninges, dura mater, arachnoid mater, or pia mater of the brain. Intracerebral administration may include, in some cases, administration of an agent into the cerebrospinal fluid (CSF) of the subarachnoid space surrounding the brain.

In some cases, intracerebral administration involves injection using stereotaxic procedures. Stereotaxic procedures are well known in the art and typically involve the use of a computer and a 3-dimensional scanning device that are used together to guide injection to a particular intracerebral region, e.g., a ventricular region. Micro-injection pumps (e.g., from World Precision Instruments) may also be used. In some cases, a microinjection pump is used to deliver a composition comprising a viral vector. In some cases, the infusion rate of the composition is in a range of 0.1 μl/min to 100 μl/min. As will be appreciated by the skilled artisan, infusion rates will depend on a variety of factors, including, for example, species of the subject, age of the subject, weight/size of the subject, serotype of the AAV, dosage required, and intracerebral region targeted. Thus, other infusion rates may be deemed by a skilled artisan to be appropriate in certain circumstances.

Delivery of rAAVs to a subject may be accomplished, for example, by intravenous administration. In certain instances, it may be desirable to deliver the rAAVs locally to the brain tissue, the spinal cord, cerebrospinal fluid (CSF), neuronal cells, glial cells, meninges, astrocytes, oligodendrocytes, microglia, interstitial spaces, and the like. In some cases, recombinant AAVs may be delivered directly to the CNS by injection into the ventricular region, as well as to the parenchyma, striatum, substantia nigra, cortex, cerebellar lobule, thalamus, hippocampus or other brain region or combination of brain regions. AAVs may be delivered with a needle, catheter or related device, using neurosurgical techniques known in the art, such as by stereotactic injection (see, e.g., Stein et al., J Vir. (1999) 73:3424−9; Davidson et al., PNAS. (2000) 97:3428-32; Davidson et al., Nat Genet. (1993) 3:219-223; and Alisky and Davidson, Hum. Gene Ther. (2000) 11:2315-29.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of neurology, medicine, medicinal and pharmaceutical chemistry, and cell biology described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

In order that this invention may be better understood, the following examples are set forth. These examples are for purposes of illustration only and are not to be construed as limiting the scope of the invention in any manner.

### Examples

### Example 1: Screening of ZFP-TFs

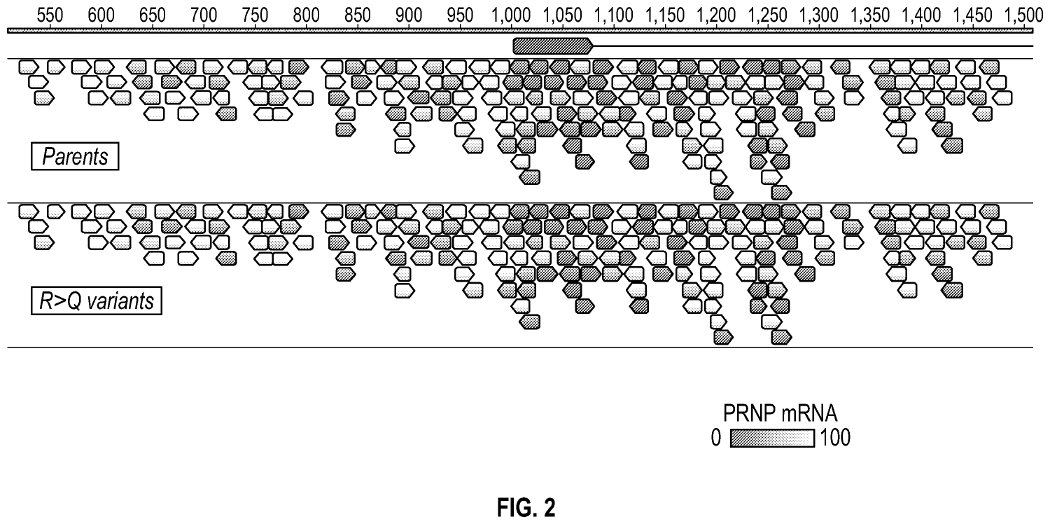

In order to identify ZFP-TFs that repress the expression of the mouse PRNP gene, we designed and screened a library of 384 ZFP-TFs predicted to bind 15 or up to 22 bp sequences in the region of the mouse PRNP gene spanning from about 500 bp upstream of the transcription start site (TSS) to about 500 bp downstream of the TSS (FIGS. 1 and 2). The target regions of the ZFP-TFs are denoted by arrowheads in the two figures, with the direction of the arrowhead indicating the strand of DNA that the ZFP-TF binds to (5′ to 3′). 192 of the ZFP-TFs were parental proteins, while the other half were variants of the parental proteins containing three R-to-Q mutations (Miller et al., Nat Biotechnol. (2019) 37(8):945−52). In this study, a KRAB domain sequence (SEQ ID NO: 262) was used as the transcription repressor and fused to the C-terminus of the ZFP domain.

Messenger RNA encoding each ZFP-TF was produced and aliquoted into 96-well plates in a 6-dose dilution. Mouse Neuro2a cells were transfected with the mRNA using the Amaxa® Nucleofector® device (Lonza, Switzherland). After 24 hours, total RNA was extracted from the cells and the expression of PRNP and two reference genes (ATP5b, EIF4A2) was monitored using real-time RT-qPCR. To do so, the cells were lysed and reverse transcription was performed using the C2CT kit following the manufacturer's instructions. TaqMan quantitative polymerase chain reaction (qPCR) was used to measure the expression levels of PRNP. PRNP expression levels were normalized to the geometric mean of the expression levels of the housekeeping genes EIF4A2 and ATP5B. A mock transfection and transfection with a ZFP-TF known not to target PRNP were used as negative controls.

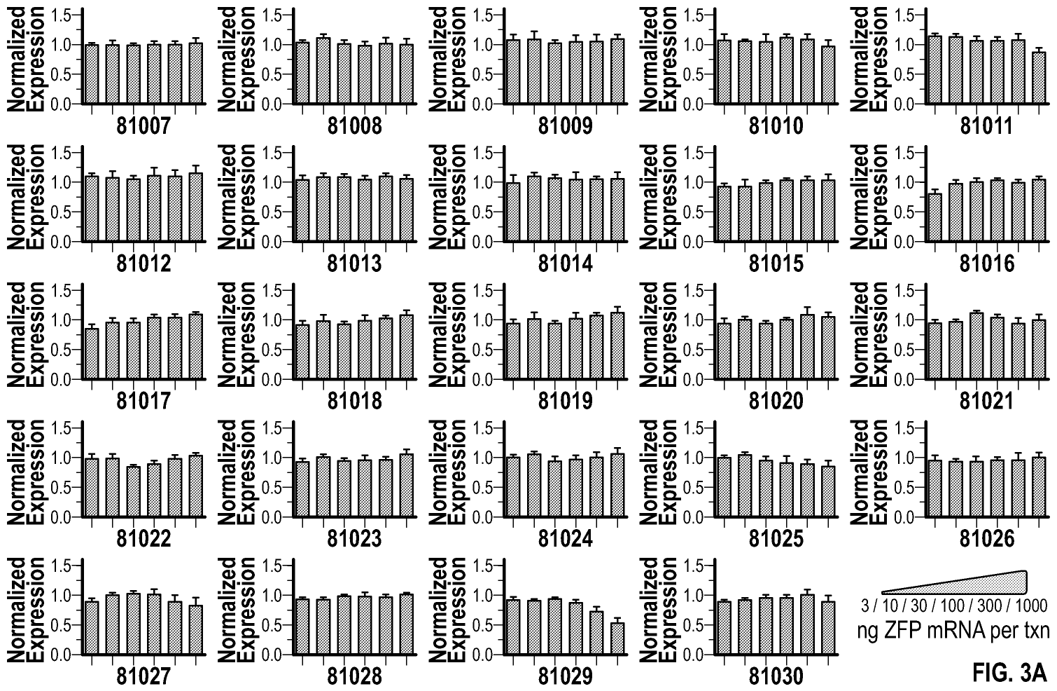

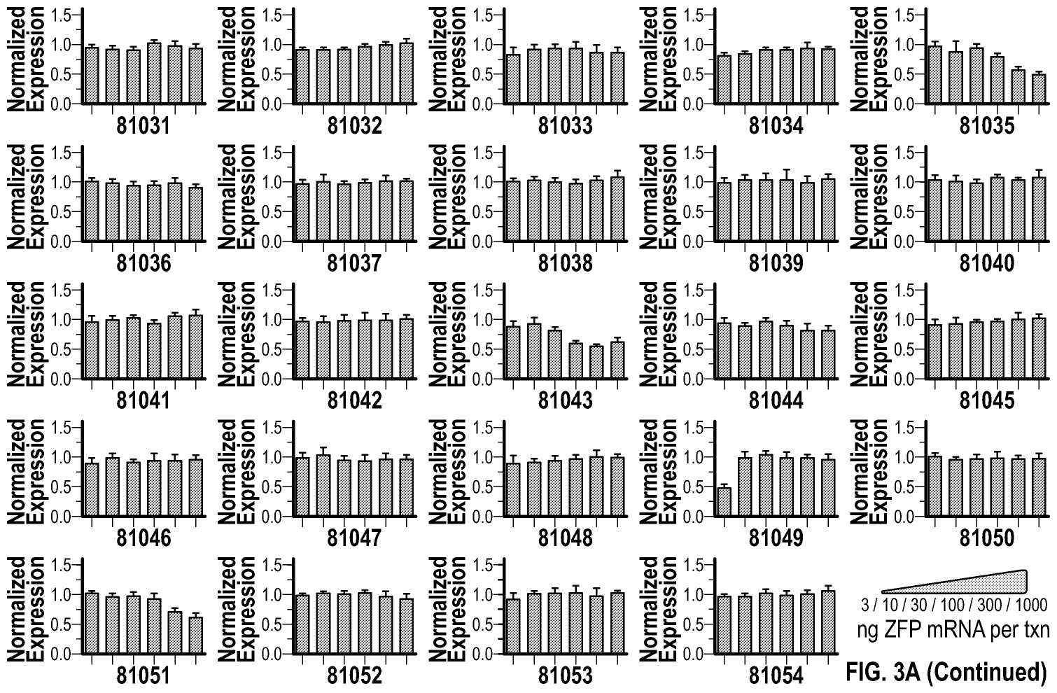

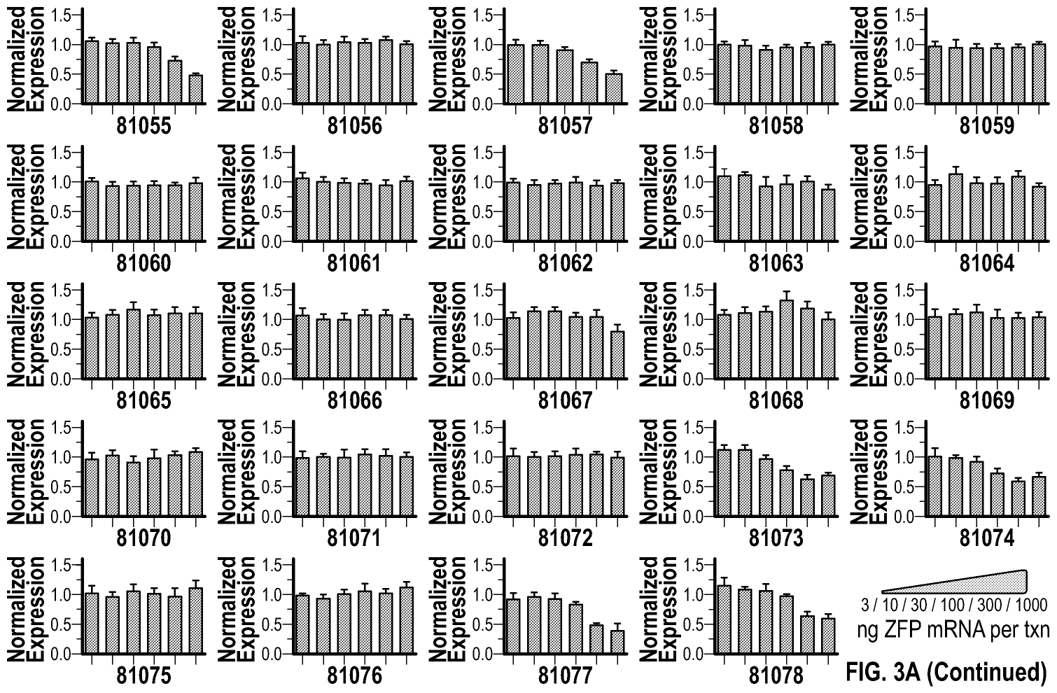

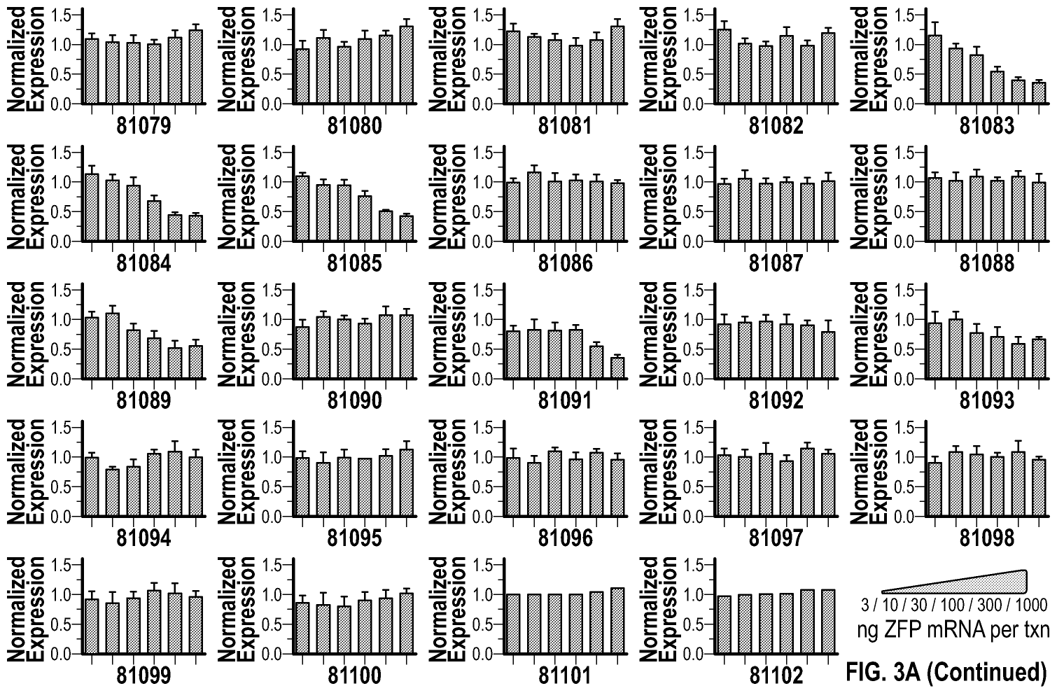

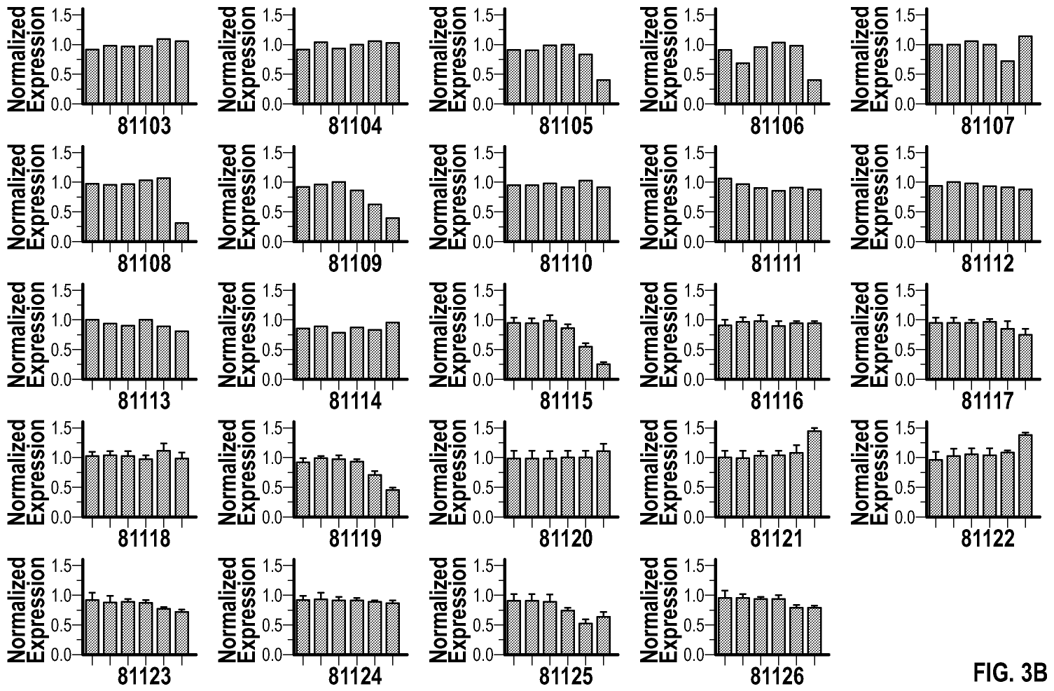

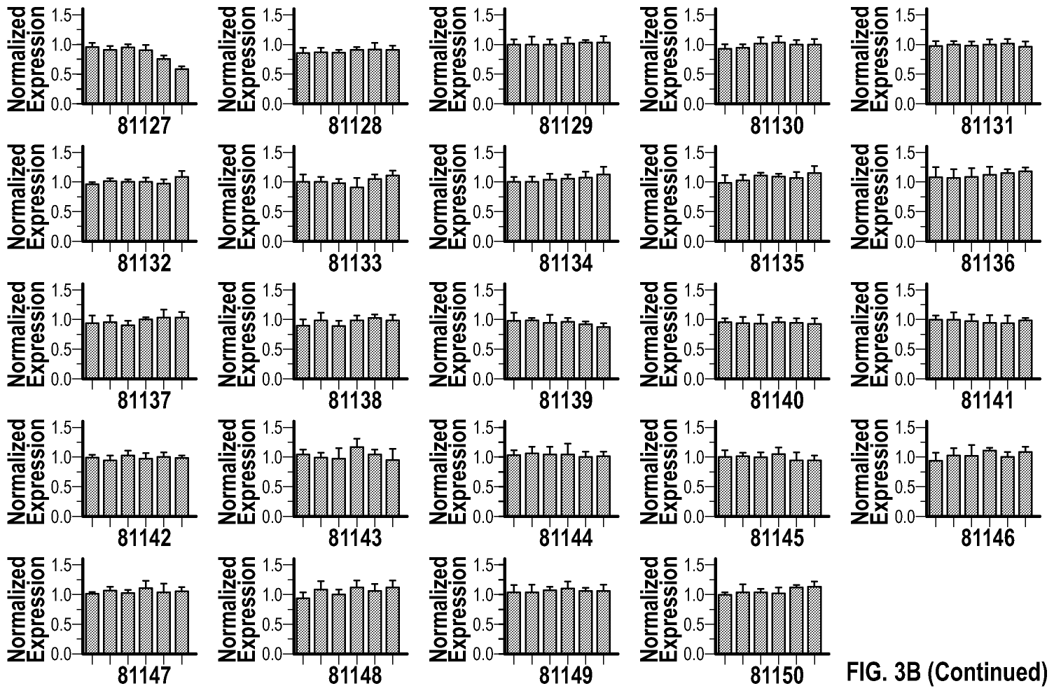

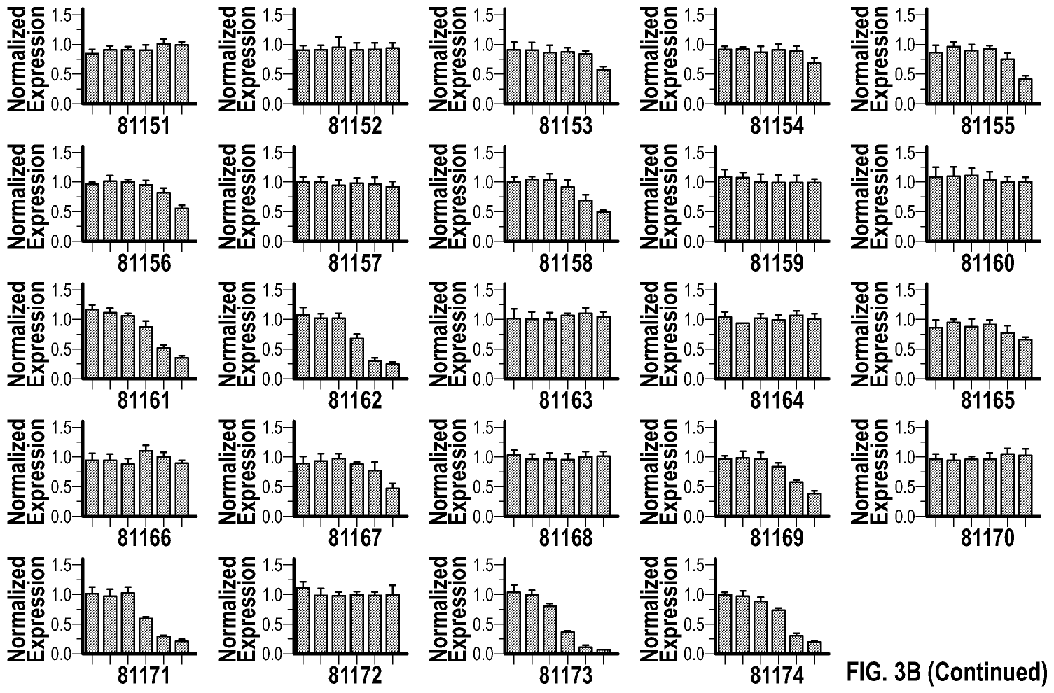

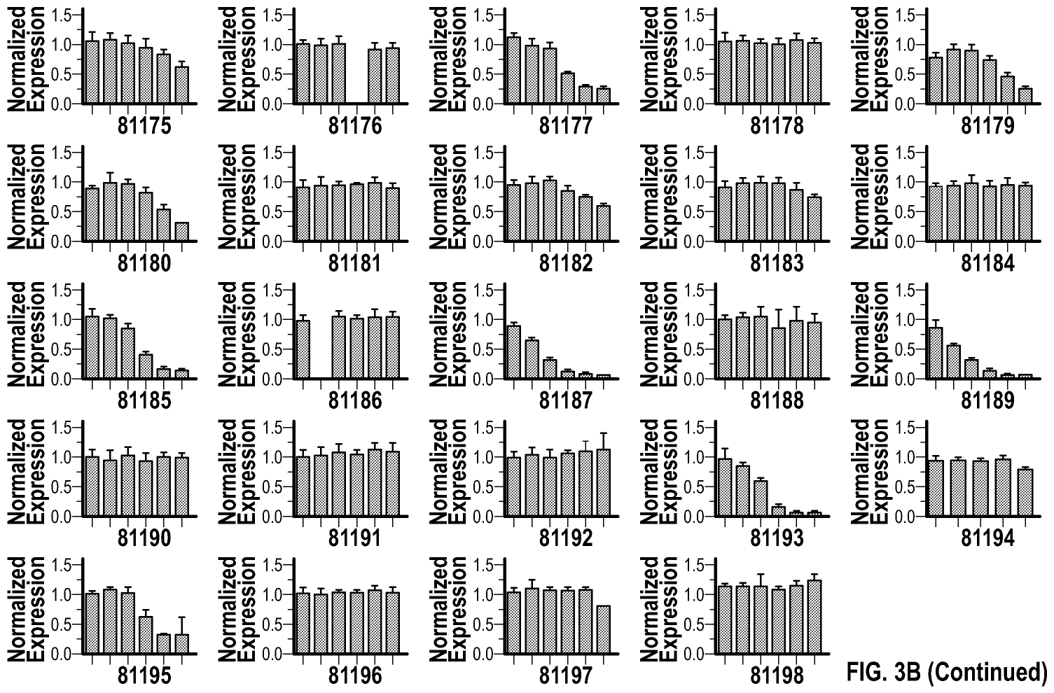

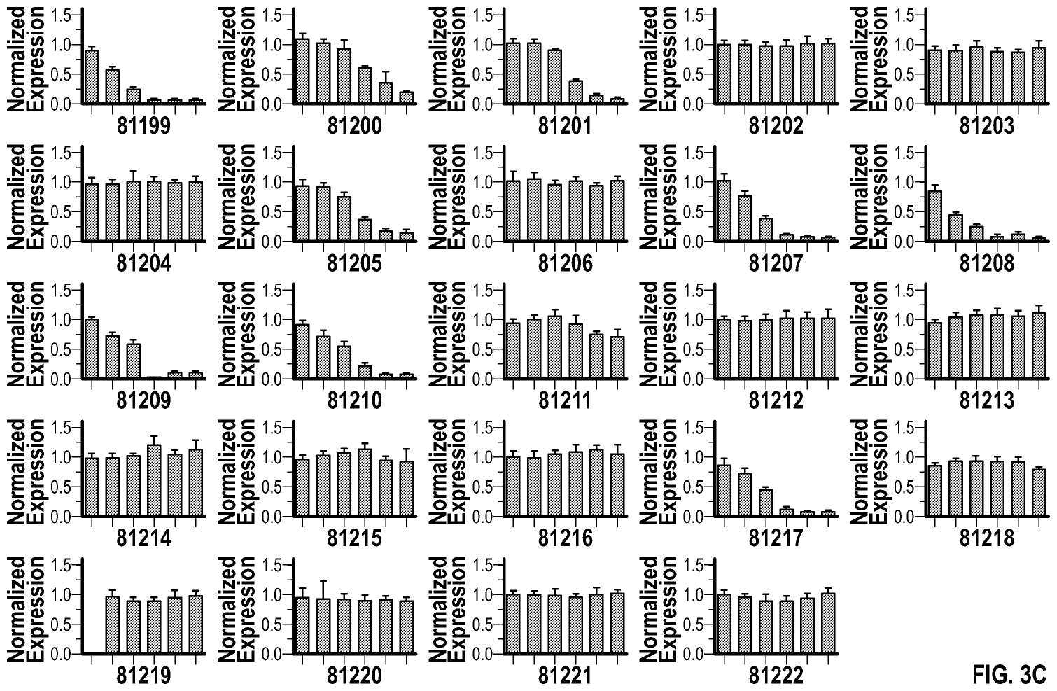

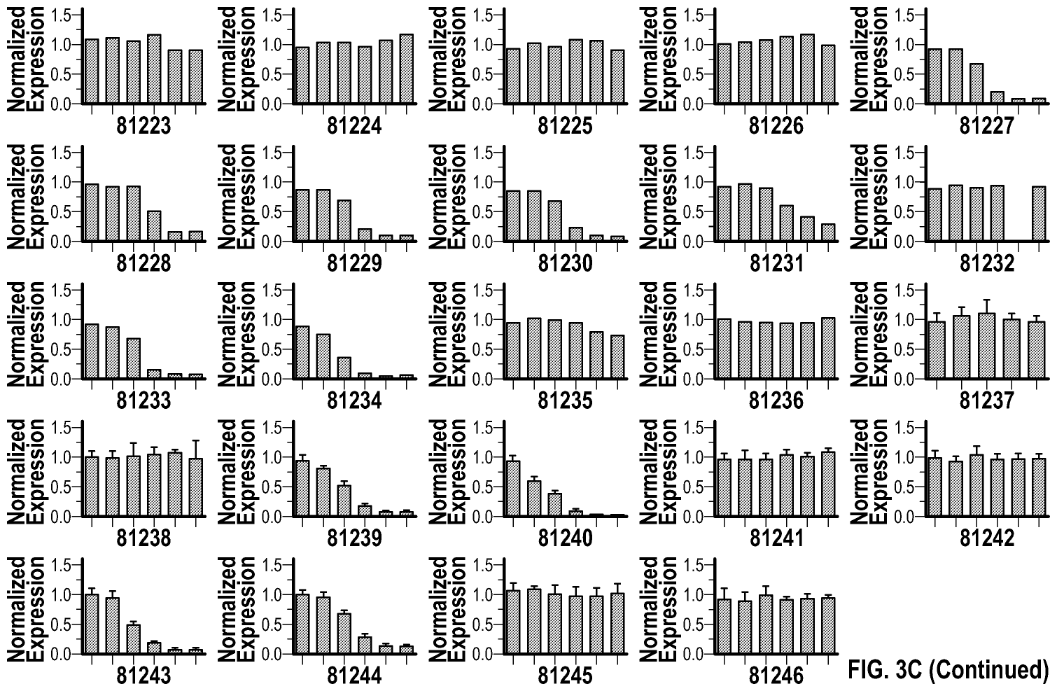

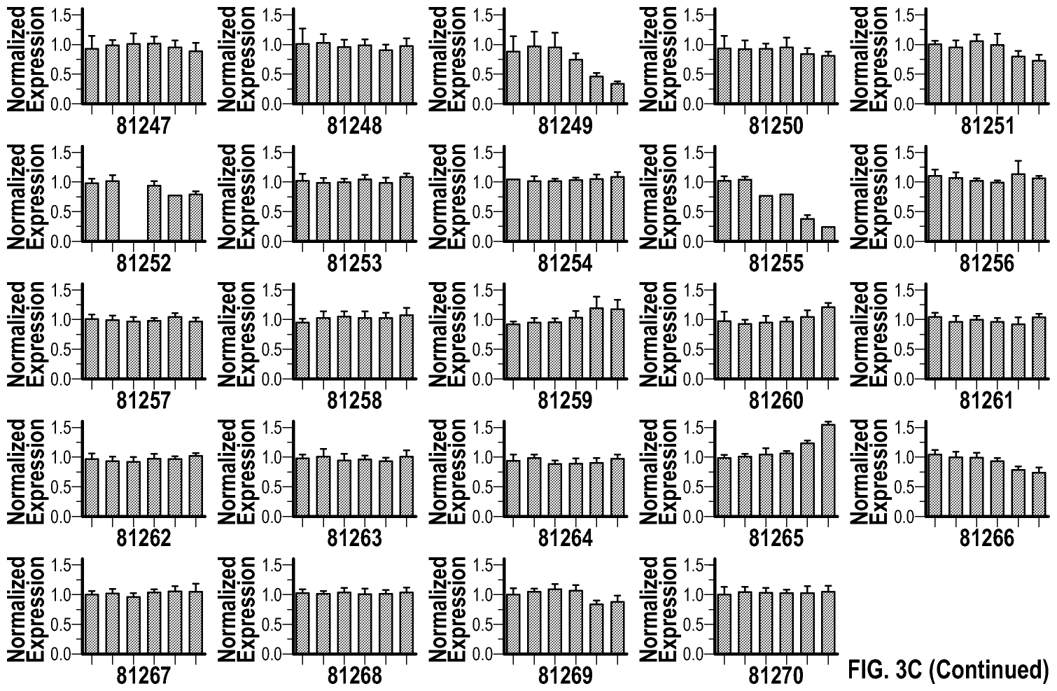

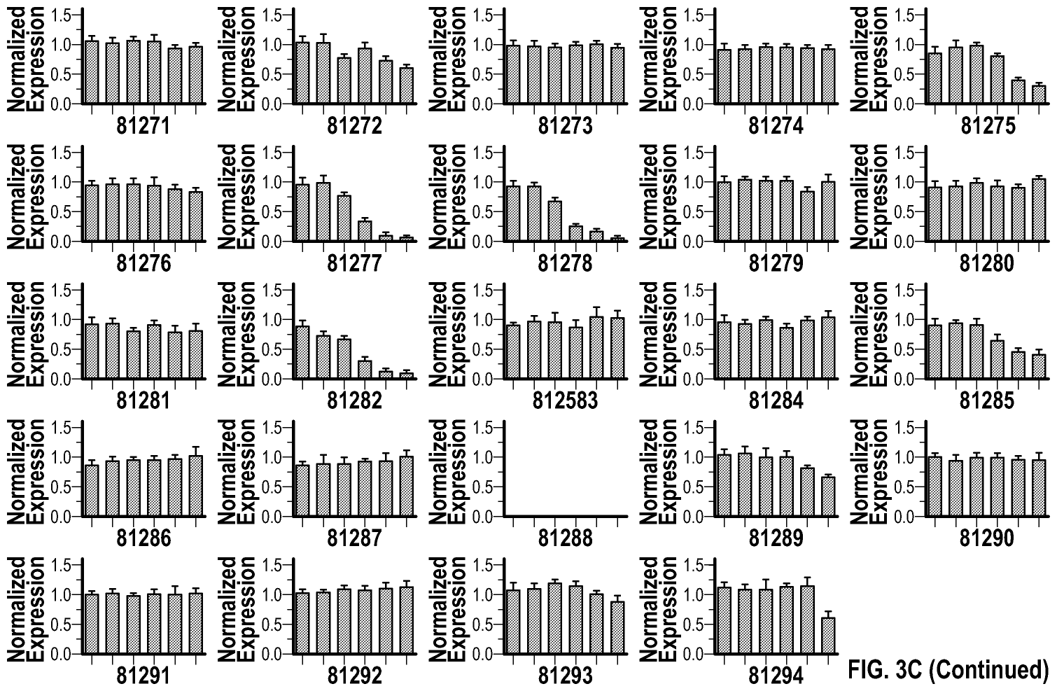

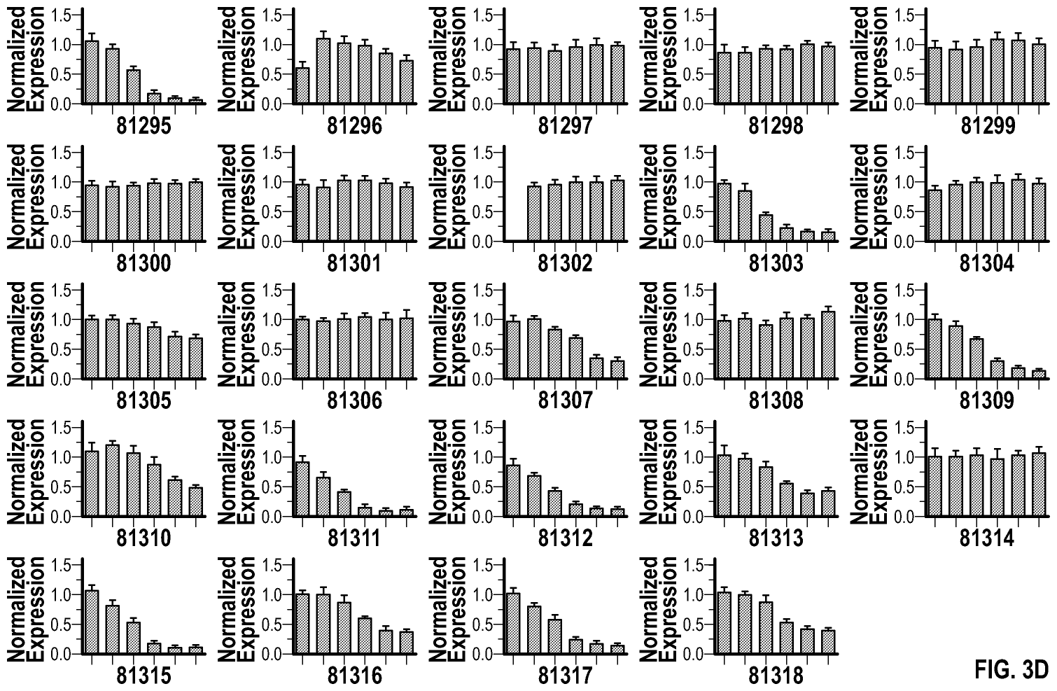

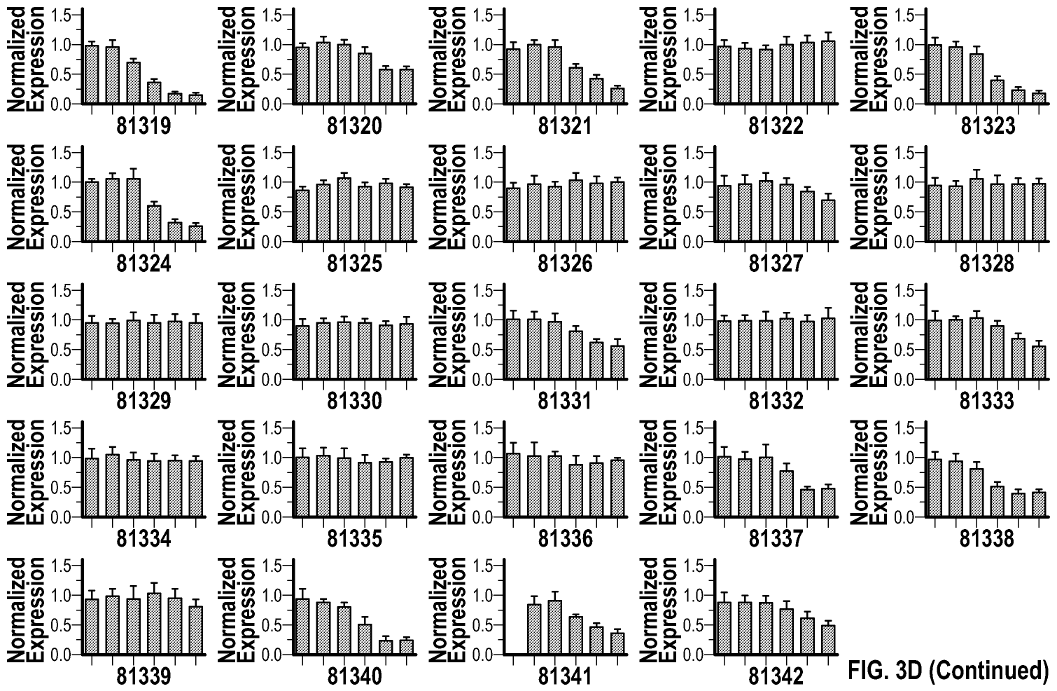

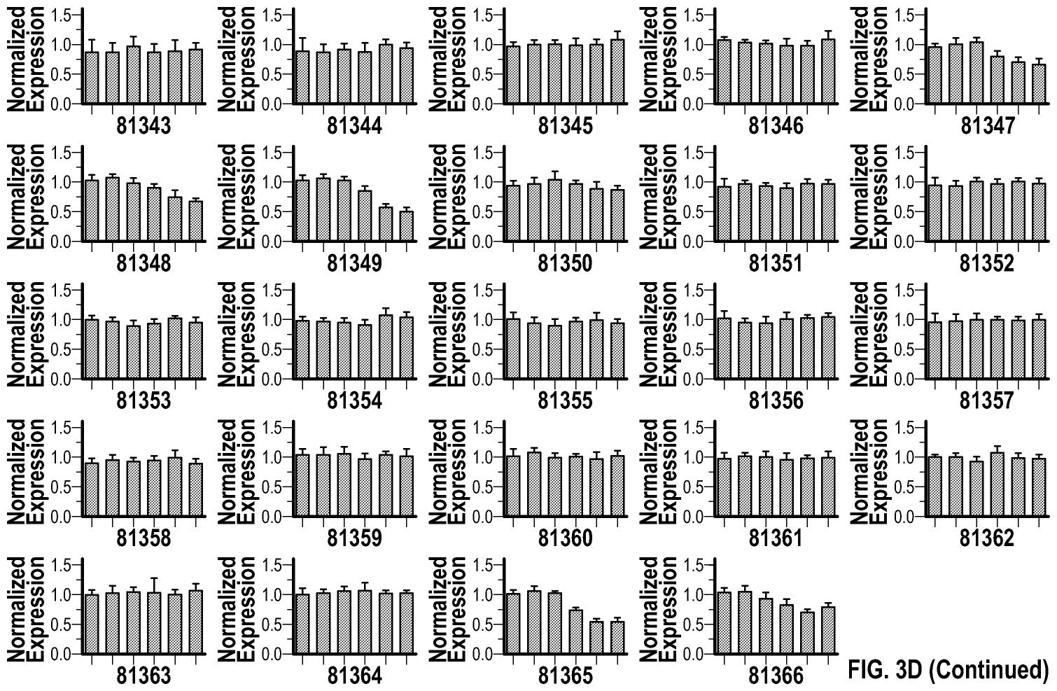

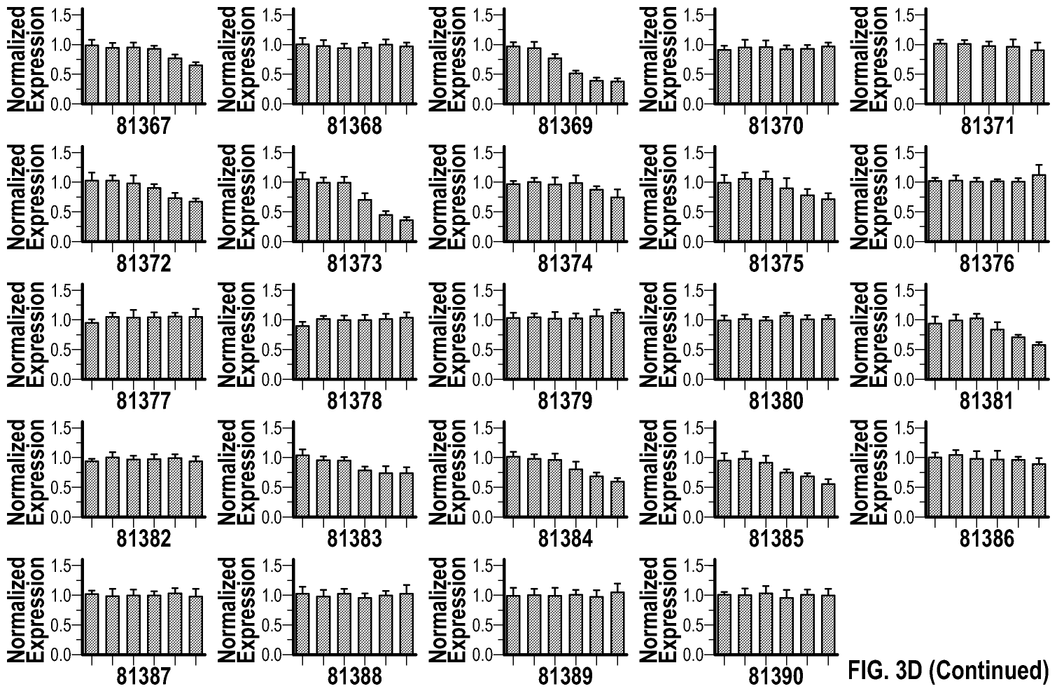

FIG. 3A-D show normalized PRNP expression 24 hours after administration of the indicated amounts of the ZFP-TFs in mRNA form (at the indicated ZFP-TF mRNA doses of 3 ng, 10 ng, 30 ng, 100, ng, 300 ng or 1000 ng, left to right, respectively). The ZFP-TFs' ability to repress PRNP expression is also indicated by the color gradient in FIG. 2, where the more potent the ZFP-TF, the darker its color.

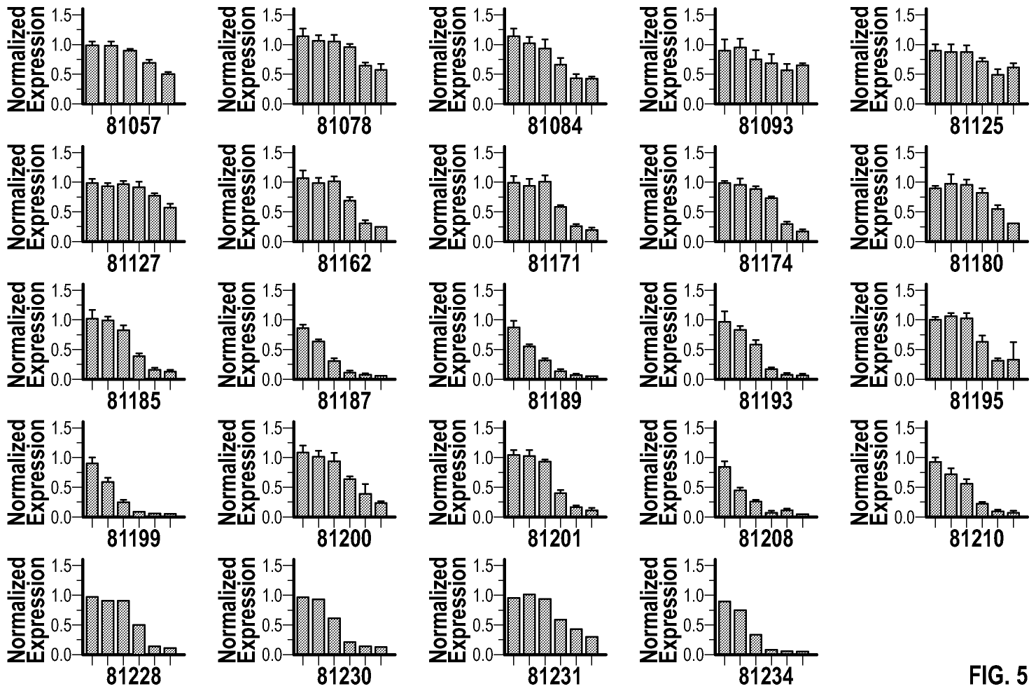

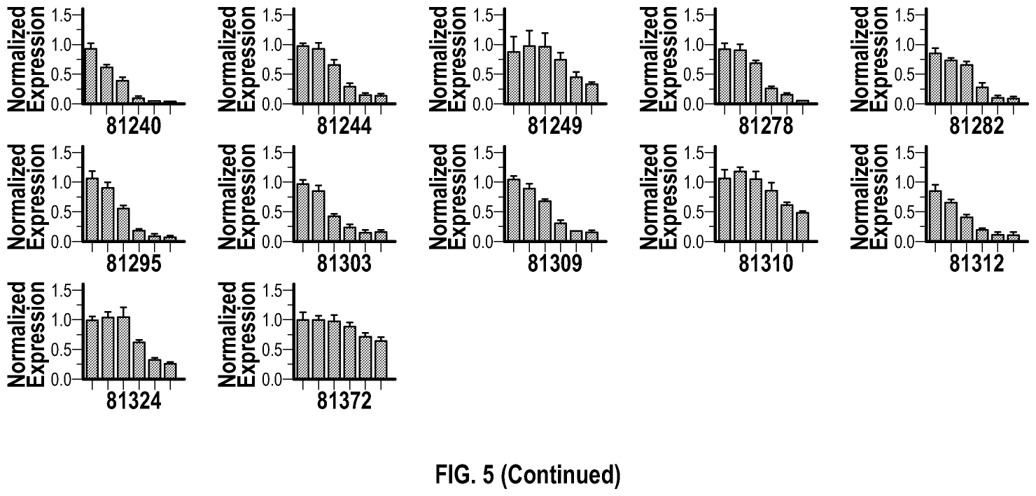

Out of the 384 ZFP-TFs, 36 were selected for further studies. The mouse PRNP genomic sequences targeted by these 36 ZFP-TFs, as well as the DNA-binding amino acid sequences of the six zinc fingers in the ZFP-TFs, are shown in FIG. 4. Full amino acid sequences of the corresponding ZFPs are shown in FIG. 9A. The activity of each of these 36 ZFP-TFs in Neuro2a cells is shown FIG. 5. The data in FIG. 5 indicate that ZFP-TF ##81185, 81187, 81189, 81193, 81199, 81201, 81208, 81210, 81228, 81230, 81234, 81240, 81244, 81278, 81282, 81295, 81303, 81309, and 81312 were especially potent, demonstrating dose-dependent repressing effects on the expression of mouse PRNP gene.

### Example 2: PRNP-Repressing Activity of Selected ZFP-TFs in Primary Neurons

We next tested the activity of the 36 selected ZFP-TFs in mouse primary cortical neurons. Primary mouse cortical neurons (MCNs; Gibco) were cultured according to the manufacturer's protocol. Coding sequences for the ZFP-TFs were cloned into recombinant AAV2/6 vectors using a human SYN1 promoter to drive expression. Virus was produced in HEK293T cells, purified using a CsCl density-gradient, and titrated by real time qPCR according to methods known in the art. The purified virus was used to infect cultured primary MCNs on DIV2 at 1×10² vg/cell, 3×10² vg/cell, 1×10³ vg/cell, 3×10³ vg/cell, 1×10⁴ vg/cell, or 3×10⁴ vg/cell. After seven days, total RNA was extracted from the neurons and the expression of PRNP mRNA and three reference genes (ATP5b, EIF4A2, and GAPDH) were monitored using real-time RT-qPCR.

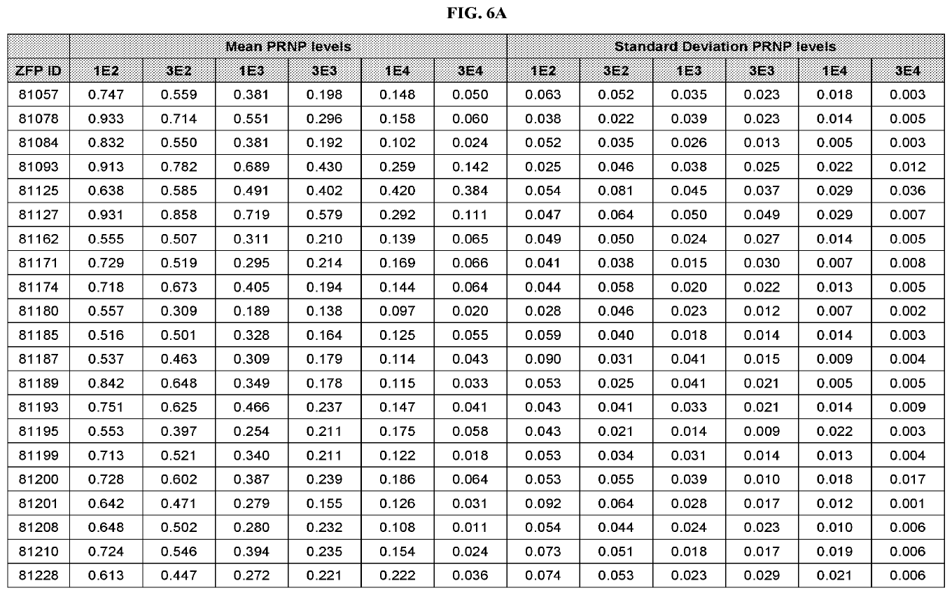

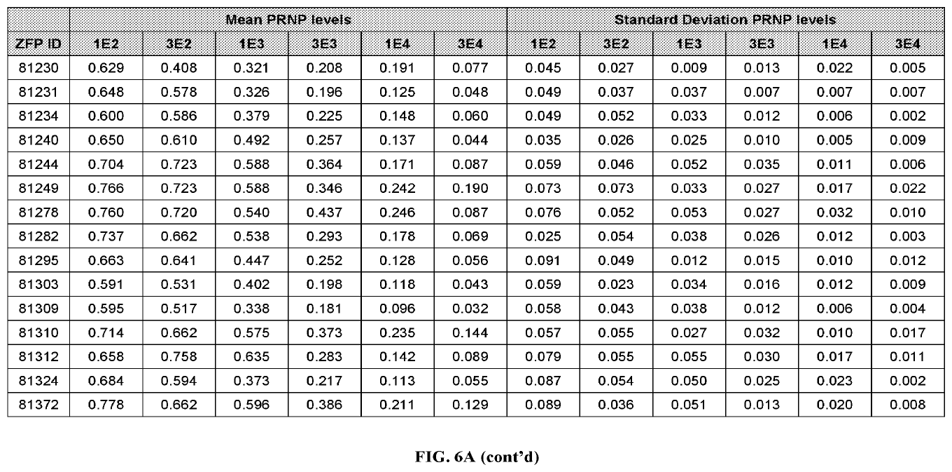

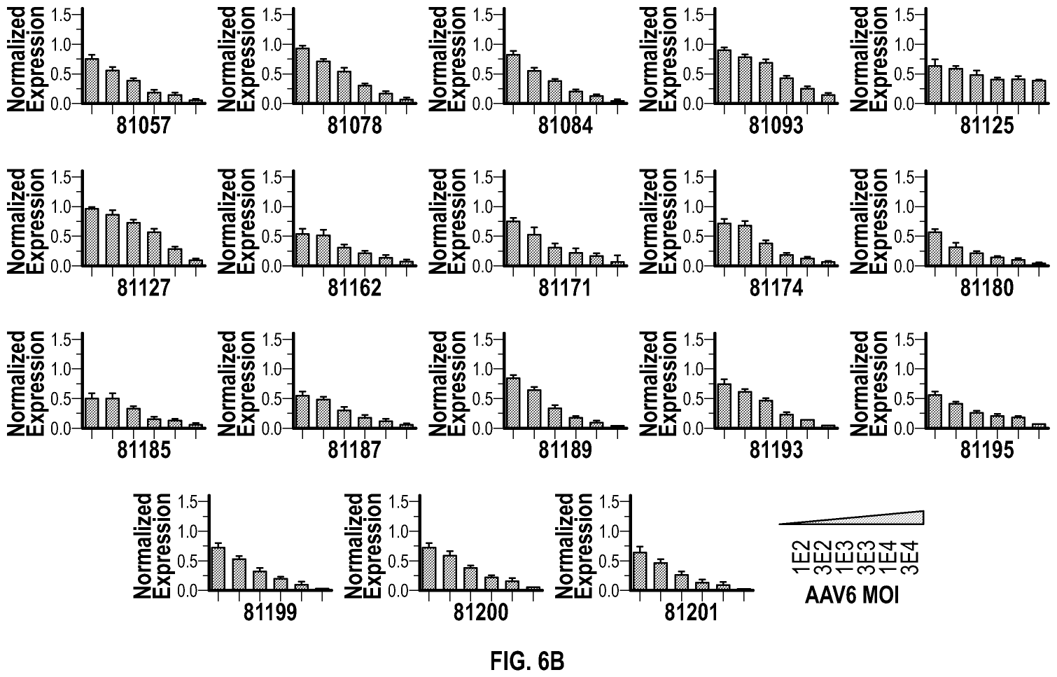

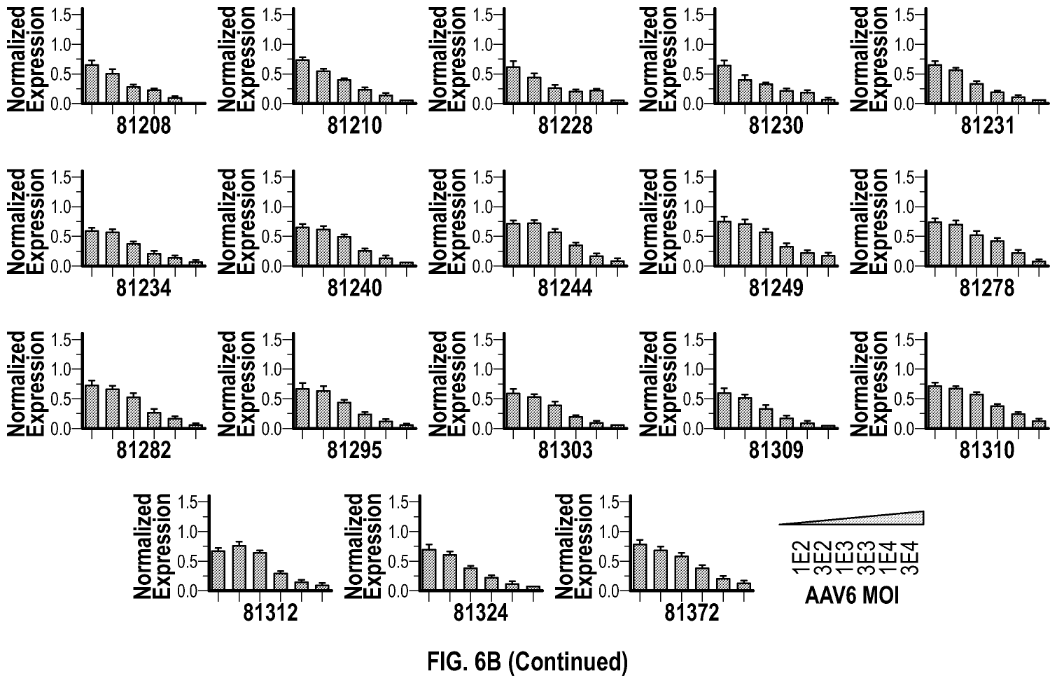

The data show that all 36 selected ZFP-TFs demonstrated robust dose-dependent repressing activity on PRNP expression in the mouse neurons (FIGS. 6A and 6B).

### Example 3: Off-Target Activity of Mouse PRNP ZFP-TFs

To evaluate the off-target impact of the mouse Prnp ZFP-TFs on global gene expression, we performed microarray experiments on total RNA isolated from primary mouse cortical neurons treated with AAV6s encoding the representative prion ZFP-TFs.

Primary mouse cortical neurons were purchased from Gibco. Cells were plated onto poly-D-lysine-coated 24-well plates at 200,000 cells/well and maintained according to the manufacturer's specifications using Gibco Neurobasal Medium containing GlutaMAX™ I supplement, B27 supplement, and penicillin/streptomycin. Forty-eight hours after plating (at DIV2), the cells were infected with AAV6 at a multiplicity of infection (MOI) of 3E3 VGs/cell and harvested 7 days later (at DIV9; 50% media exchanges performed every 3-4 days). This was followed by RNA isolation and microarray analysis.

Off-target analysis was performed using the GeneTitan™ platform (Clariom S kit) according to the manufacturer's instructions. The assay results were analyzed using TAC software. Genes were considered differentially regulated for FDR-corrected p-values ≤0.05. A ZFP-TF known to have minimal off-targets and a mock transfection were used as negative controls.

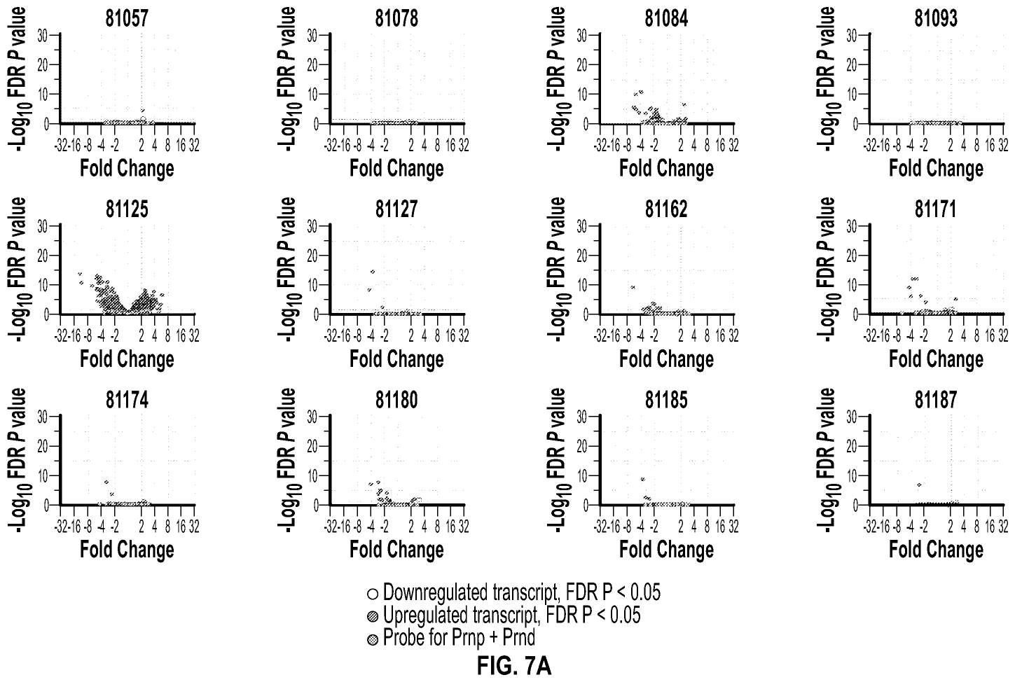

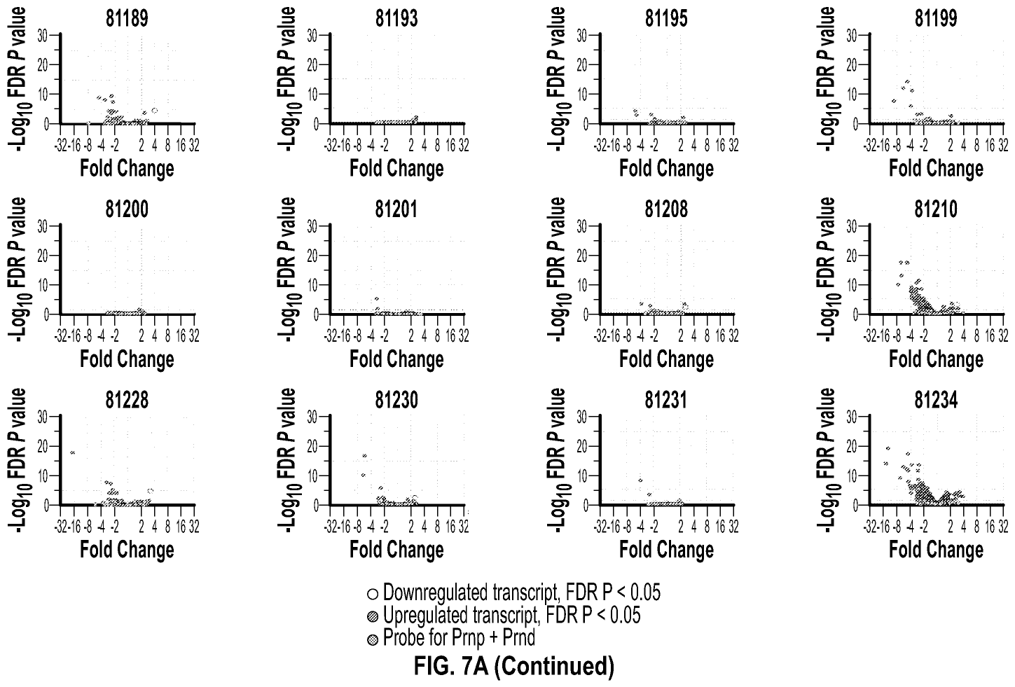

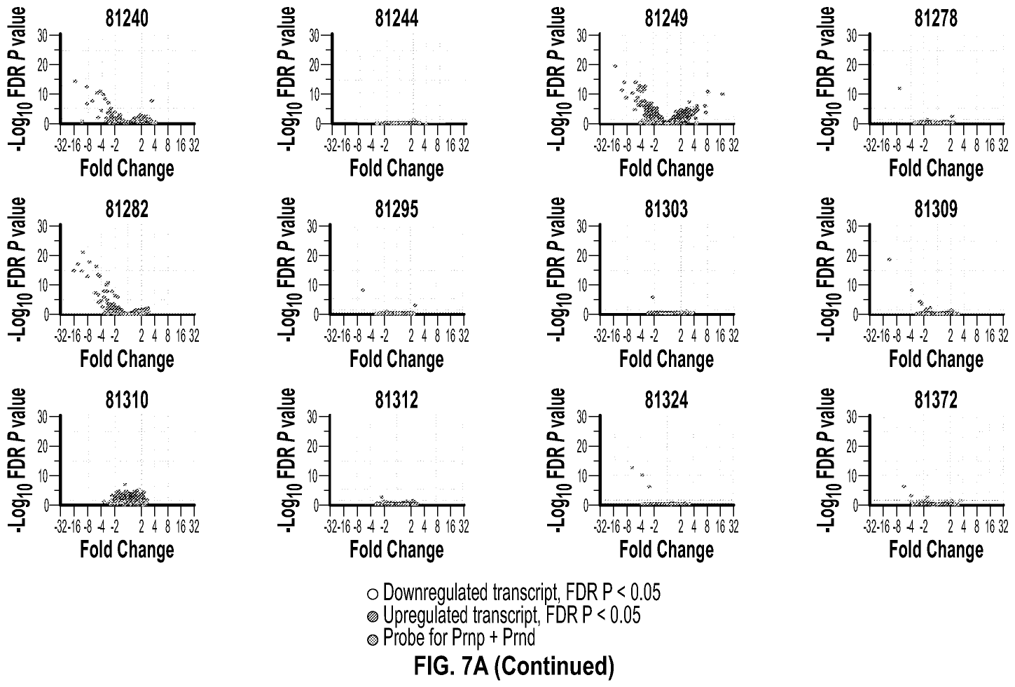

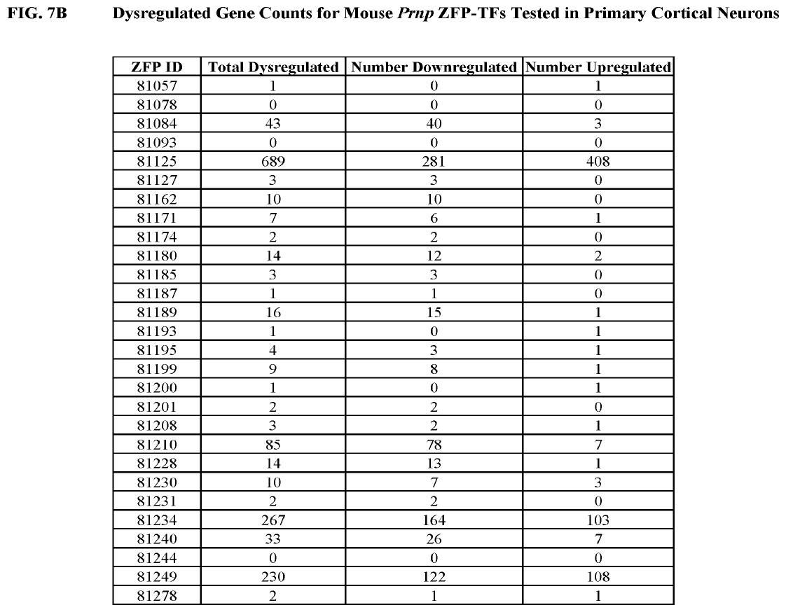

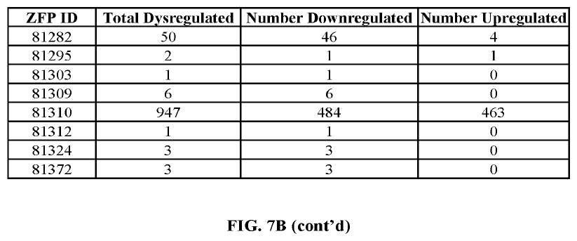

FIG. 7A shows the microarray results of 36 representative mouse PRNP ZFP-TFs tested in primary mouse cortical neurons. A range of off-target specificities were observed, with some ZFP-TFs displaying very low to no off-target activity. FIG. 7B shows the number of dysregulated for each ZFP-TF.

### Example 4: Activity of Human PRNP ZFP-TFs in Human iPSC-Derived Neurons

Twelve ZFP-TFs designed to target human PRNP were tested in human iPSC-derived GABAergic neurons (Cellular Dynamics International). The human PRNP genomic sequences targeted by these 12 ZFP-TFs, as well as the DNA-binding amino acid sequences of the zinc fingers in the ZFP-TFs, are shown in FIG. 8A. Full amino acid sequences of the corresponding ZFPs are shown in FIG. 9B. The cells were plated onto poly-L-ornithine- and laminin-coated 96-well plates at a density of 40,000 cells per well and then maintained according to the manufacturer's instructions. The cells were transfected with AAV6 expressing the desired ZFP-TF at 6 different MOI (1E3, 3E3, 1E4, 3E4, 1E5, and 3E5) 48 hours after plating. The transduced cells were maintained for up to 33 days (50-75% media changes performed every 3−5 days). The cells were harvested after 31 days following AAV infection.

Harvested cells were lysed and reverse transcription was performed using the C2CT kit following the manufacturer's instructions. TaqMan quantitative polymerase chain reaction (qPCR) was used to measure the expression levels of PRNP. PRNP expression levels were normalized to the geometric mean of the expression levels of the housekeeping genes EIF4A2, ATP5B and GAPDH. A mock infection was used as negative control.

Dose-dependent repression was demonstrated with the ZFP-TFs targeting human PRNP. The maximum repression achieved was more than 99%, but ZFP-TFs that repressed prion to a lesser degree were also identified (e.g., about 90%, about 75%, or about 80% at the highest dose).

The dose-dependent activities of the 12 exemplary ZFP-TFs targeting human PRNP are shown in FIGS. 8B and 8C. These data show that the ZFP-TFs display a range of prion repression activity profiles, with prion mRNA repression at the highest dose tested ranging from about 75% to greater than 99%.

### Example 5: Off-Target Activity of Human PRNP ZFP-TFs

To evaluate the off-target impact of the human PRNP ZFP-TFs on global gene expression, we performed microarray experiments on total RNA isolated from human iPSC-derived neurons treated with AAVs encoding representative human PRNP ZFP-TFs.

Human iPSC-derived neurons were treated as described in Example 4. For microarray analysis, the cells were plated onto poly-L-ornithine- and laminin-coated 24-well plates at a density of 260,000 cells per well, transduced with AAV6 expressing the desired ZFP-TF at 1E5 VGs/cell 48 hours after plating, and harvested 19 days after viral transfection. Total RNA was isolated from the harvested cells and used for microarray analysis.

Off-target analysis was performed using the GeneTitan™ platform (Clariom S kit) according to the manufacturer's instructions. The assay results were analyzed using TAC software. Genes were considered differentially regulated for FDR-corrected p-values ≤0.05. A ZFP-TF known to have minimal off-targets and a mock transfection were used as negative controls.

FIG. 8D shows the microarray results of 12 human PRNP ZFP-TFs tested in human iPSC-derived neurons. A range of off-target specificities were observed, with some ZFP-TFs displaying very low off-target activity. FIG. 8E shows the number of dysregulated genes for each ZFP-TF.

1. A fusion protein comprising a zinc finger protein (ZFP) domain and a transcription repressor domain, wherein the ZFP domain binds to a target region of a mammalian prion protein gene (PRNP gene).

2. The fusion protein of claim 1, wherein the target region is within about 1 kb or 500 bp of a transcription start site (TSS) in the PRNP gene.

3. The fusion protein of claim 1 or 2, wherein the PRNP gene is a human, non-human primate, rodent, or murine PRNP gene.

4. The fusion protein of any one of the preceding claims, wherein the ZFP domain comprises six zinc fingers and optionally represses expression of the PRNP gene by at least about 40%, 75%, 90%, 95%, or 99% with minimal to no detectable off-target binding or activity.

5. The fusion protein of any one of the preceding claims, wherein the transcription repressor domain comprises a KRAB domain amino acid sequence of KOX1.

6. The fusion protein of any one of the preceding claims, wherein the ZFP domain is linked to the transcription repressor domain through a peptide linker.

7. The fusion protein of any one of the preceding claims, wherein the ZFP domain comprises a DNA-binding recognition helix sequence as shown in the tables in FIGS. 4 and 8A.

8. The fusion protein of any one of the preceding claims, wherein the ZFP domain comprises the DNA-binding recognition helix sequences as shown in a single row in the tables in FIGS. 4 and 8A.

9. A nucleic acid construct comprising a coding sequence for the fusion protein of any one of claims 1-8, wherein the coding sequence is linked operably to a transcription regulatory element.

10. The nucleic acid construct of claim 9, wherein the transcription regulatory element is a mammalian promoter that is constitutively active or inducible in a brain cell, and wherein the promoter is optionally a human synapsin I promoter.

11. A host cell comprising the nucleic acid construct of claim 9 or 10.

12. The host cell of claim 11, wherein the host cell is a human cell.

13. The host cell of claim 11, wherein the host cell is a brain cell or a pluripotent stem cell, wherein the stem cell is optionally an embryonic stem cell or an inducible pluripotent stem cell (iPSC).

14. A recombinant virus comprising the nucleic acid construct of claim 9 or 10.

15. The recombinant virus of claim 14, wherein the recombinant virus is recombinant adeno-associated virus (AAV), optionally of serotype 6 or 9.

16. A pharmaceutical composition comprising the nucleic acid construct of claim 9 or 10, or the recombinant virus of claim 14 or 15, and a pharmaceutically acceptable carrier.

17. A method of inhibiting expression of prion protein (PrP) in a mammalian brain cell, comprising introducing into the cell a fusion protein of any one of claims 1-6, optionally through introduction of a nucleic acid construct of any one of claim 7 or 8 or the recombinant virus of claim 14 or 15, thereby inhibiting the expression of PrP in the cell.

18. The method of claim 17, wherein the mammalian brain cell is a human, non-human primate, rodent, or murine cell.

19. The method of claim 17 or 18, wherein the mammalian brain cell is a neuron, a glial cell, an ependymal cell, or a neuroepithelial cell.

20. The method of any one of claims 17-19, wherein the cell is in the brain of a patient suffering from or at risk of developing prion disease, wherein the prion disease is optionally familial, sporadic, or acquired prion disease.

21. The method of claim 20, wherein the prion disease is Creutzfeldt-Jakob Disease (CJD), sporadic CJD, variant CJD, Gerstmann-Straussler-Scheinker Syndrome (GSS), Fatal Familial Insomnia (FFI), sporadic Fatal Insomnia (sFI), Kuru, or variably protease-sensitive prionopathy (VPSPr).

22. The method of any one of claims 17-21, comprising introducing into the cell the recombinant virus of claim 14 or 15.

23. A method of treating or preventing a neurodegenerative disease in a patient, comprising administering to the patient a recombinant AAV of claim 15.

24. The method of claim 23, wherein the neurodegenerative disease is prion disease, optionally wherein the prion disease is familial, sporadic, or acquired prion disease.

25. The method of claim 23 or 24, wherein the AAV is introduced to the patient via intravenous, intrathecal, intracerebroventrical, intra-cisternal magna, or intrathalamic injection, or injection into any cerebral region.

26. The method of claim 24 or 25, wherein the prion disease is Creutzfeldt-Jakob Disease (CJD), sporadic CJD, variant CJD, Gerstmann-Straussler-Scheinker Syndrome (GSS), Fatal Familial Insomnia (FFI), sporadic Fatal Insomnia (sFI), Kuru, or variably protease-sensitive prionopathy (VPSPr).

27. A fusion protein of any one of claims 1-8, a nucleic acid construct of claim 9 or 10, or a recombinant virus of claim 14 or 15 for use in the method of any one of claims 17-26.

28. Use of a fusion protein of any one of claims 1-8, a nucleic acid construct of claim 9 or 10, or a recombinant virus of claim 14 or 15 for the manufacture of a medicament for treating a patient in the method of any one of claims 17-26.
